(19) United States
(12) Patent Application Publication
(10) Pub. No.: US 2008/0139551 A1
(43) Pub. Date: Jun. 12, 2008

(54) TUMOR NECROSIS FACTOR ALPHA INHIBITORS AND THEIR USE IN THE TREATMENT OF HUMAN DISEASES

(75) Inventors: Jagadish Sircar, San Diego, CA (US); Sunil Kumar, San Diego, CA (US); Timothy James Davis, San Diego, CA (US); Wenbin Ying, San Diego, CA (US); Peter Nussbaumer, Vienna (AT); Andreas Billich, Vienna (AT)

Correspondence Address: KNOBBE MARTENS OLSON & BEAR LLP, 2040 MAIN STREET, FOURTEENTH FLOOR, IRVINE, CA, 92614, US

(73) Assignees: AVANIR PHARMACEUTICALS, San Diego, CA (US); NOVARTIS PHARMA GMBH, Vienna (AT)

(21) Appl. No.: 11/842,144

(22) Filed: Aug. 21, 2007

Related U.S. Application Data
(63) Continuation of application No. PCT/US2007/006874, filed on Mar. 20, 2007.
(60) Provisional application No. 60/798,059, filed on May 5, 2006.
(60) Provisional application No. 60/801,816, filed on May 19, 2006.

(30) Foreign Application Priority Data
Mar. 21, 2006 (GB) 0605689.9

(51) Int. Cl. A61K 31/5377; A61K 31/497; A61P 11/06; A61P 5/46; A61P 19/02

(52) U.S. Cl. 514/235.2; 514/253.07; 514/253.04; 514/252.11

(57) ABSTRACT

treatment of a variety of disorders, including the treatment of pathological conditions associated with tumor necrosis factor alpha. The inhibitors of tumor necrosis factor alpha have the following structures:

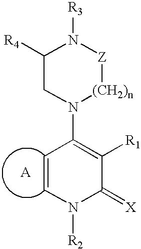

including stereoisomers, pharmaceutically acceptable salts, and solvates thereof, wherein substituents are as defined herein. Compositions containing an inhibitor of tumor necrosis factor alpha in combination with a pharmaceutically acceptable carrier are also provided, as well as methods for use of the same.

### FIELD OF THE INVENTION

Inhibitors of tumor necrosis factor alpha are provided which have utility in the treatment of a variety of disorders, including the treatment of pathological conditions associated with tumor necrosis factor alpha.

### BACKGROUND OF THE INVENTION

Tumor necrosis factor-alpha (TNF-alpha, or TNF-α) is a pleiotropic inflammatory cytokine. TNF-alpha is a member of a family of cytokines which also includes leukemia inhibitory factor (LIF), ciliary neurotrophic factor (CNTF), Oncostatin M, and cardiotrophin-1 (CT-1). All known members of the TNF-alpha cytokine family induce hepatic expression of acute phase proteins. TNF-alpha is produced by many different cell types. The main sources in vivo are stimulated monocytes, fibroblasts, and endothelial cells. Macrophages, T-cells and B-lymphocytes, granulocytes, smooth muscle cells, eosinophils, chondrocytes, osteoblasts, mast cells, glial cells, and keratinocytes also produce TNF-alpha after stimulation. Glioblastoma cells constitutively produce TNF-alpha and the factor can be detected also in the cerebrospinal fluid and human milk.

Physiological stimuli for the synthesis of TNF-alpha are interleukin-1 (IL-1), bacterial endotoxins, tumor necrosis factor (TNF), platelet-derived growth factor (PDGF), and Oncostatin M. In fibroblasts, the synthesis of TNF-alpha is stimulated by beta-interferon (IFN-beta), TNF-alpha, PDGF, and viral infections. In thymic stromal cells the synthesis of TNF-alpha can be induced by nerve growth factor (NGF). TNF-alpha can also stimulate or inhibits its own synthesis, depending upon the cell type. In epithelial, endothelial, and fibroblastic cells secretion of TNF-alpha is induced by interleukin-17 (IL-17). TNF-alpha is a 17-26 kDa protein of 185 amino acids glycosylated at positions 73 and 172. It is found as both soluble and membrane-bound forms, the active form usually being a homotrimer (Janeway et al., 1999). It is synthesized as a precursor protein of 212 amino acids. Monocytes express at least five different molecular forms of TNF-alpha with molecular masses of 21.5-28 kDa. They mainly differ by post-translational alterations such as glycosylation and phosphorylation. The human TNF-alpha gene has a length of approximately 5 kb and contains five exons. It maps to human chromosome 7p21-p14 between the markers D7S135 and D7S370. The murine gene maps to chromosome 5. The nucleotide sequences of TNF-alpha and G-CSF genes resemble each other in a way suggesting a possible evolutionary relationship.

TNF-alpha was first isolated by Carswell et al. in 1975 in an attempt to identify tumor necrosis factors responsible for necrosis of the sarcoma Meth A. Most organs of the body appear to be affected by TNF-alpha, and the cytokine serves a variety of functions, many of which are not yet fully understood. The cytokine possesses both growth stimulating properties and growth inhibitory processes, and it appears to have self regulatory properties as well. For instance, TNF-alpha induces neutrophil proliferation during inflammation, but it also induces neutrophil apoptosis upon binding to the TNF-R55 receptor. The cytokine is produced by several types of cells, but especially by macrophages.

Two beneficial functions of TNF-alpha which have lead to its continued expression may include that low levels of the cytokine may aid in maintaining homeostasis by regulating the body's circadian rhythm, and low levels of TNF-alpha promote the remodeling or replacement of injured and senescent tissue by stimulating fibroblast growth. Additional beneficial functions of TNF-alpha may include its role in the immune response to bacterial, and certain fungal, viral, and parasitic invasions as well as its role in the necrosis of specific tumors. It also acts as a key mediary in the local inflammatory immune response. TNF-alpha is an acute phase protein which initiates a cascade of cytokines and increases vascular permeability, thereby recruiting macrophage and neutrophils to a site of infection. TNF-alpha secreted by macrophages causes blood clotting which serves to contain the infection.

While TNF-alpha may have beneficial functions, TNF-alpha also exhibits pathological activities. Although TNF-alpha causes necrosis of some types of tumors, it promotes the growth of other types of tumor cells. High levels of TNF-alpha correlate with increased risk of mortality, and TNF-alpha participates in both inflammatory disorders of inflammatory and non inflammatory origin. Sepsis was believed to result directly from the invading bacteria itself, but it was later recognized that host system proteins, such as TNF-alpha, induced sepsis in response. Exogenous and endogenous factors from bacteria, viruses, and parasites stimulate production of TNF-alpha and other cytokines. Lipopolysaccharide from bacteria cell walls is an especially potent stimulus for TNF-alpha synthesis. When cytokine production increases to such an extent that it escapes the local infection, or when infection enters the bloodstream, sepsis ensues. Systematic edema results in low blood volume, hypoproteinanemia, neutropenia, and then neutrophilia. Body organs fail and death may result. Victims of septic shock experience fever, falling blood pressure, myocardial suppression, dehydration, acute renal failure, and then respiratory arrest.

TNF-alpha exhibits chronic effects as well as resulting in acute pathologies. If TNF-alpha remains in the body for a long time, it loses its anti-tumor activity. This can occur due to polymerization of the cytokine, shedding of TNF receptors by tumor cells, excessive production of anti-TNF antibodies as is observed in patients with carcinomas or chronic infection, and disruptions in the alpha-2 macroglobulin proteinase system which may deregulate cytokines. Prolonged overproduction of TNF-alpha also results in a condition known as cachexia, which is characterized by anorexia, net catabolism, weight loss, and anemia and which occurs in illnesses such as cancer and AIDS.

### SUMMARY OF THE INVENTION

As TNF-alpha has been identified in a variety of tissues and has been associated with numerous pathological events, there exists a need in the art to identify inhibitors of TNF-alpha. There is also a need for pharmaceutical compositions containing such inhibitors, as well as methods relating to the use thereof to treat TNF-alpha induced pathological events. The preferred embodiments may fulfill these needs, and provide other advantages as well.

In preferred embodiments, inhibitors of TNF-alpha are provided that have the following general structure including forms such as stereoisomers, free forms, pharmaceutically acceptable salts or esters thereof, solvates, or combinations of such forms, wherein the substituents are as defined below:

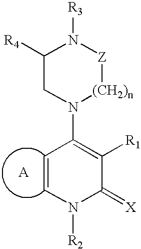

The TNF-alpha inhibitors of preferred embodiments have utility over a wide range of therapeutic applications, and may be employed to treat a variety of disorders, illnesses, or pathological conditions including, but not limited to, septic shock, cancer, AIDS, transplantation rejection, multiple sclerosis, diabetes, rheumatoid arthritis, trauma, malaria, meningitis, ischemia-reperfusion injury, adult respiratory distress syndrome, and others. Such methods include administering an effective amount of one or more TNF-alpha inhibitors as provided by the preferred embodiments, preferably in the form of a pharmaceutical composition, to a patient in need thereof. Pharmaceutical compositions are provided containing one or more TNF-alpha inhibitors of preferred embodiments in combination with a pharmaceutically acceptable carrier and/or diluent.

TNF-alpha exerts its action mainly through the two TNF-receptors, TNF receptor I (renamed CD120a) and TNF receptor II (renamed CD120b). The majority of the TNF-alpha effects are transmitted through CD120a, whereas the CD120b receptor is inducible and preferentially reacts with membrane bound TNF-alpha (Tartaglia et al., 1992, Vandenabeele et al., 1995, Grell et al., 1995). The biological function of TNF-alpha has been well investigated and organic compounds which interfere with TNF-alpha activity, e.g., which inhibit TNF-alpha activity, have been described to be useful in the treatment of numerous disorders (diseases).

Inflammatory cytokines such as TNF have been implicated in the pathogenesis of psoriasis (Bonifati and Ameglio, Int. J. Derm. 38: 241-251, 1999). Leonardi et al. (New Eng. J. Med. 349: 2014-2022, 2003) found that treatment with the TNF antagonist etanercept led to a significant reduction in the severity of psoriasis over a treatment period of 24 weeks. Boyman et al. (J. Exp. Med. 199: 731-736, 2004) engrafted keratome biopsies of human symptomless prepsoriatic skin onto AGR129 mice, which are deficient in type I and type II interferon receptors, as well as Rag2, and thereby lack B and T cells and show severely impaired NK cell activity. Upon engraftment, human T cells underwent local proliferation, which was crucial for development of a psoriatic phenotype exhibiting papillomatosis and acanthosis. Immunohistochemical analysis of prepsoriatic skin before transplantation and 8 weeks after transplantation showed activation of epidermal keratinocytes, dendritic cells, endothelial cells, and immune cells in the transplanted tissue. T-cell proliferation and the subsequent disease development were dependent on TNF production and could be inhibited by antibody or soluble receptor to TNF. Boyman et al. concluded that TNF-dependent activation of resident T cells is necessary and sufficient for development of psoriatic lesions.

Studies in mice (Flynn et al., Immunity 2: 561-572, 1995) and observations in patients receiving infliximab (remicade) for treatment of rheumatoid arthritis or Crohn's disease (IBD1; (Keane et al., N. Eng. J. Med. 345: 1098-1104, 2001) have shown that antibody-mediated neutralization of TNF increases susceptibility to tuberculosis. However, excess TNF may be associated with severe TB pathology (Barnes et al., J. Immun. 145: 149-154, 1990). Using path and segregation analysis and controlling for environmental differences, Stein et al. (Hum. Hered. 60: 109-118, 2005) evaluated TNF secretion levels in Ugandan TB patients. The results suggested that there is a strong genetic influence, due to a major gene, on TNF expression in TB, and that there may be heterozygote advantage. The effect of shared environment on TNF expression in TB was minimal. Stein et al. concluded that TNF is an endophenotype for TB that may increase power to detect disease-predisposing loci.

Single-nucleotide polymorphisms (SNPs) in regulatory regions of cytokine genes have been associated with susceptibility to a number of complex disorders. TNF is a proinflammatory cytokine that provides a rapid form of host defense against infection but is fatal in excess. Because TNF is employed against a variety of pathogens, each involving a different pattern of risks and benefits, it might be expected that this would favor diversity in the genetic elements that control TNF production.

Herrmann et al. (Europ. J. Clin. Invest. 28: 59-66, 1998) used PCR-SSCP and sequencing to screen the entire coding region and 1,053 bp upstream of the transcription start site of the TNF-alpha gene for polymorphisms. Five polymorphisms were identified: 4 were located in the upstream region at positions −857, −851, −308, and −238 from the first transcribed nucleotide, and 1 was found in a nontranslated region at position +691. Three SNPs located at nucleotides −238, −308, and −376 with respect to the TNF transcriptional start site are all substitutions of adenine for guanine. Knight et al. (Nature Genet. 22: 145-150, 1999) referred to the allelic types as −238G/−238A, −308G/−308A, and −376G/−376A. They stated that variation in the TNF-alpha promoter region had been found to be associated with susceptibility to cerebral malaria (McGuire et al., Nature 371: 508-511, 1994), with mucocutaneous leishmaniasis (Cabrera et al., J. Exp. Med. 182: 1259-1264, 1995), with death from meningococcal disease (Nadel et al., J. Infect. Dis. 174: 878-880, 1996), with lepromatous leprosy (Roy et al., J. Infect. Dis. 176: 530-532, 1997), with scarring trachoma (Conway et al., Infect. Immun. 65: 1003-1006, 1997), and with asthma (Moffatt and Cookson, Hum. Molec. Genet. 6: 551-554, 1997).

Flori et al. (Hum. Molec. Genet. 12: 375-378, 2003) tested for linkage between polymorphisms within the NMHC region and mild malaria. Two-point analysis indicated linkage of mild malaria to TNFd (lod=3.27), a highly polymorphic marker in the NMC region. Multipoint analysis also indicated evidence for linkage of mild malaria to the NMC region, with a peak close to TNF (lod=3.86). The authors proposed that genetic variation within TNF may influence susceptibility to mild malaria, but the polymorphisms TNF-238, TNF-244, and TNF-308 are unlikely to explain linkage of mild malaria to the NMC region.

Statistical analyses by Funayama et al. (Invest. Ophthal. Vis. Sci. 45: 4359-4367, 2004) showed a possible interaction between polymorphisms in the optineurin and TNF genes that would increase the risk for the development and probably progression of glaucoma in Japanese patients with POAG.

By sequencing the promoter regions 500 bp upstream from the transcriptional start sites of members of the TNF and TNFR superfamilies, Kim et al. (Immunogenetics 57: 297-303, 2005) identified 23 novel regulatory SNPs in Korean donors. Sequence analysis suggested that 9 of the SNPs altered putative transcription factor binding sites. Analysis of SNP databases suggested that the SNP allele frequencies were similar to those for Japanese subjects but distinct from those of Caucasian or African populations.

Zinman et al. (J. Clin. Endocr. Metab. 84: 272-278, 1999) studied the relationship between TNF-alpha and anthropometric and physiologic variables associated with insulin resistance and diabetes in an isolated Native Canadian population with very high rates of NIDDM. Using the homeostasis assessment (HOMA) model to estimate insulin resistance, they found moderate, but statistically significant, correlations between TNF-alpha and fasting insulin, HOMA insulin resistance, waist circumference, fasting triglycerides, and systolic blood pressure; in all cases, coefficients for females were stronger than those for males. The authors concluded that in this homogeneous Native Canadian population, circulating TNF-alpha concentrations were positively correlated with insulin resistance across a spectrum of glucose tolerance. The data suggested a possible role for TNF-alpha in the pathophysiology of insulin resistance.

Rasmussen et al. (J. Clin. Endocr. Metab. 85: 1731-1734, 2000) investigated whether the −308 and −238 G-to-A genetic variants of TNF were associated with features of the insulin resistance syndrome or alterations in birth weight in 2 Danish study populations comprising 380 unrelated young healthy subjects and 249 glucose-tolerant relatives of type 2 diabetic patients, respectively. Neither of the variants was related to altered insulin sensitivity index or other features of the insulin resistance syndrome. Birth weight and the ponderal index were also not associated with the polymorphisms. Their study did not support a major role of the −308 or −238 substitutions in TNF in the pathogenesis of insulin resistance or altered birth weight among Danish Caucasian subjects.

Obayashi et al. (J. Clin. Endocr. Metab. 85: 3348-3351, 2000) investigated the influence of TNF-alpha on the predisposition to insulin dependency in adult-onset diabetic patients with type I diabetes (IDDM)-protective HLA haplotypes. The TNF-alpha of 3 groups of DRB*1T502-DQB1*0601-positive diabetic patients who had initially been nonketotic and noninsulin dependent for more than 1 year was analyzed. Group A included II antibodies to glutamic acid decarboxylase (GADab)-positive patients who developed insulin dependency within 4 years of diabetes onset. Group B included II GADab-positive patients who remained noninsulin dependent for more than 12 years. Group C included 12 GADab-negative type 2 diabetes, and a control group included 18 nondiabetic subjects. In the group C and control subjects, DRB*1T502-DQB1*0601 was strongly associated with the TNF-alpha-13 allele. DRB1*1502-DQB1*0601 was strongly associated with the TNF-alpha −12 allele among the group A patients, but not among the group B patients. Interestingly, sera from all patients with non-TNF-alpha −12 and non-TNF-alpha −13 in group B reacted with GAD65 protein by Western blot. The authors concluded that TNF-alpha is associated with a predisposition to progression to insulin dependency in GADab/DRB1*1502-DQB1*0601-positive diabetic patients initially diagnosed with type II diabetes and that determination of these patients' TNF-alpha genotype may allow for better prediction of their clinical course.

To study whether the TNF-alpha gene could be a modifying gene for diabetes, Li et al. (J. Clin. Endocr. Metab. 88: 2767-2774, 2003) studied TNF-alpha promoter polymorphisms (G-to-A substitution at positions −308 and −238) in relation to HLA-DQB1 genotypes in type 2 diabetes patients from families with both type I and type 2 diabetes (type 1/2 families) or common type 2 diabetes families as well as in patients with adult-onset type I diabetes and control subjects. The TNF-alpha (308) AA/AG genotype frequency was increased in adult-onset type 1 patients (55%, 69 of 126), but it was similar in type 2 patients from type 1/2 families (35%, 33/93) or common type 2 families (31%, 122 of 395), compared with controls (33%, 95/284; P less than 0.0001 versus type 1). The TNF-alpha (308) A and DQB1*02 alleles were in linkage disequilibrium in type 1 patients (Ds=0.81; P less than 0.001 versus Ds=0.25 in controls) and type 2 patients from type 1/2 families (Ds=0.59, P less than 0.05 versus controls) but not in common type 2 patients (Ds=0.39). The polymorphism was associated with an insulin-deficient phenotype in type 2 patients from type 1/2 families only together with DQB*02, whereas the common type 2 patients with AA/AG had lower waist-to-hip ratio [0.92 (0.12) versus 0.94 (0.11), P=0.008] and lower fasting C-peptide concentration [0.48 (0.47) versus 0.62 (0.46) nmol/liter, P=0.020] than those with GG, independently of the presence of DQB1*02. The authors concluded that TNF-alpha is unlikely to be the second gene on the short arm of chromosome 6 responsible for modifying the phenotype of type 2 diabetic patients from families with both type 1 and type 2 diabetes.

Shbaklo et al. (Hum. Immunol. 64: 633-638, 2003) evaluated TNF-alpha promoter polymorphisms at positions −863 and −1031 and their association with type 1 diabetes in a group of 210 diabetic patients in Lebanon. Their results showed that in that population, the C allele is predominant at position −863, whereas the A allele is rare (2%). At position −1031, however, the C and T allele distribution was similar in both the patient (17.8% versus 82.2%, respectively) and the control (21.4% versus 79.6%) groups. No association of TNF-alpha genotype at position 1031 with type 1 diabetes was found as demonstrated by the family-based association test and the transmission disequilibrium test. However, when patient genotypes were compared, the recessive CC genotype was found in type 1 diabetic males but not in type 1 diabetic females.

From studies of 641 patients with myocardial infarction and 710 control subjects, Herrmann et al. (Europ. J. Clin. Invest. 28: 59-66, 199) concluded that polymorphisms of the TNF-alpha gene are unlikely to contribute to coronary heart disease risk in an important way, but that the −308 mutation should be investigated further in relation to obesity.

Because TNF-alpha expression had been reported to be increased in adipose tissue of both rodent models of obesity and obese humans, TNF-alpha was considered a candidate gene for obesity. Norman et al. (J. Clin. Invest. 96: 158-162, 1995) scored Pima Indians for genotypes at 3 polymorphic dinucleotide repeat loci near the TNF-alpha gene. In a sib-pair linkage analysis, the percentage of body fat, as measured by hydrostatic weighing, was linked (304 sib pairs, P=0.002) to the marker closest (10 kb) to TNF-alpha. The same marker was associated (P=0.01) by analysis of variants with body mass index (BMI). To search for DNA variants in TNF-alpha possibly contributing to obesity, they performed SSCP analysis on the gene from 20 obese and 20 lean subjects. No association could be demonstrated between alleles at the single polymorphism located in the promoter region and percent of body fat.

Rosmond et al. (J. Clin. Endocr. Metab. 86: 2178-2180, 2001) examined the potential impact of the G-to-A substitution at position −308 of the TNF-alpha gene promoter on obesity and estimates of insulin, glucose, and lipid metabolism as well as circulating hormones including salivary cortisol in 284 unrelated Swedish men born in 1944. Genotyping revealed allele frequencies of 0.77 for allele G and 0.23 for allele A. Tests for differences in salivary cortisol levels between the TNF-alpha genotypes revealed that, in homozygotes for the rare allele in comparison with the other genotypes, there were significantly higher cortisol levels in the morning, before as well as 30 and 60 minutes after stimulation by a standardized lunch. In addition, homozygotes for the rare allele had a tendency toward higher mean values of body mass index, waist-to-hip ratio, and abdominal sagittal diameter compared with the other genotype groups. The results also indicated a weak trend toward elevated insulin and glucose levels among men with the A/A genotype. Rosmond et al. suggested that the increase in cortisol secretion associated with this polymorphism might be the endocrine mechanism underlying the previously observed association between the NcoI TNF-alpha polymorphism and obesity, as well as insulin resistance.

To evaluate the role of TNF-alpha in the pathogenesis of hyperandrogenism, Escobar-Morreale et al. (J. Clin. Endocr. Metab. 86: 3761-3767, 2001) evaluated the serum TNF-alpha levels, as well as several polymorphisms in the promoter region of the TNF-alpha gene, in a group of 60 hyperandrogenic patients and 27 healthy controls matched for body mass index. Hyperandrogenic patients presented with mildly increased serum TNF-alpha levels as compared with controls. When subjects were classified by body weight, serum TNF-alpha was increased only in lean patients as compared with lean controls; this difference was not statistically significant when comparing obese patients with obese controls. The TNF-alpha gene polymorphisms studied were equally distributed in hyperandrogenic patients and controls. However, carriers of the −308A variant presented with increased basal and leuprolide-stimulated serum androgens and 17-hydroxyprogesterone levels when considering patients and controls as a group. The authors concluded that the TNF-alpha system might contribute to the pathogenesis of hyperandrogenism.

De Groof et al. (J. Clin. Endocr. Metab. 87: 3118-3124, 2002) evaluated the GH/IGF1 axis and the levels of IGF-binding proteins (IGFBPs), IGFBP3 protease, glucose, insulin, and cytokines in 27 children with severe septic shock due to meningococcal sepsis during the first 3 days after admission. The median age was 22 months. Nonsurvivors had extremely high GH levels that were significantly different compared with mean GH levels in survivors during a 6-hour GH profile. Significant differences were found between nonsurvivors and survivors for the levels of total IGF1, free IGF1, IGFBP1, IGFBP3 protease activity, IL6, and TNF-alpha. The pediatric risk of mortality score correlated significantly with levels of IGFBP1, IGFBP3 protease activity, IL6, and TNF-alpha and with levels of total IGF1 and free IGF1. Levels of GH and IGFBP1 were extremely elevated in nonsurvivors, whereas total and free IGF1 levels were markedly decreased and were accompanied by high levels of the cytokines IL6 and TNF-alpha.

Mira et al. (J.A.M.A. 282: 561-568, 1999) reported the results of a multicenter case-control study of the frequency of the −308G-A polymorphism, which they called the TNF2 allele, in patients with septic shock. Eighty-nine patients with septic shock and 87 healthy unrelated blood donors were studied. Mortality among patients with septic shock was 54%. The polymorphism frequencies of the controls and patients differed only at the TNF2 allele (39% versus 18% in the septic shock and control groups, respectively, P=0.002). Among the septic shock patients, TNF2 polymorphism frequency was significantly greater among those who had died (52% versus 24% in the survival group, P=0.008). Concentrations of TNF-alpha were higher with TNF2 (68%) than with TNF1 (52%), but their median values were not statistically different. Mira et al. estimated that patients with the TNF2 allele had a 3.7-fold risk of death.

Because fatal cerebral malaria is associated with high circulating levels of tumor necrosis factor-alpha, McGuire et al. (Nature 371: 508-511, 1994) undertook a large case-control study in Gambian children. The study showed that homozygotes for the TNF2 allele, a variant of the TNF-alpha gene promoter region (Wilson et al., Hum. Molec. Genet. 1: 353 only, 1992), had a relative risk of 7 for death or severe neurologic sequelae due to cerebral malaria. Although the TNF2 allele is in linkage disequilibrium with several neighboring HLA alleles, McGuire et al. showed that this disease association was independent of HLA class I and class II variation. The data suggested that regulatory polymorphisms of cytokine genes can affect the outcome of severe infection. The maintenance of the TNF2 allele at a gene frequency of 0.16 in The Gambia implies that the increased risk of cerebral malaria in homozygotes is counterbalanced by some biologic advantage.

Hill (Proc. Assoc. Am. Phys. 111: 272-277, 1999) reviewed the genetic basis of susceptibility and resistance to malaria, and tabulated 10 genes that are known to affect susceptibility or resistance to Plasmodium falciparum and/or Plasmodium vivax. He noted that the association of an upregulatory variant of the TNF gene promoter (Wilson et al., Hum. Molec. Genet. 1: 353 only, 1992) with cerebral malaria (McGuire et al., Nature 371: 508-511, 1994) had encouraged the assessment of agents that might reduce the activity of this cytokine (van Hensbroek et al., J. Infect. Dis. 174: 1091-1097, 1996).

Through systematic DNA fingerprinting of the TNF promoter region, Knight et al. (Nature Genet. 22: 145-150, 1999) identified a SNP that causes the helix-1 turn-helix transcription factor OCT1 (POU2F1) to bind to a novel region of complex protein-DNA interactions and alters gene expression in human monocytes. The OCT1-binding genotype, found in approximately 5% of Africans, was associated with 4-fold increased susceptibility to cerebral malaria in large studies comparing cases and controls in West African and East African populations, after correction for other known TNF polymorphisms and linked HLA alleles.

Galbraith and Pandey (Hum. Genet. 96: 433-436, 1995) studied 2 polymorphic systems of tumor necrosis factor-alpha in 50 patients with alopecia greata. The first biallelic TNF-alpha polymorphism was detected in humans by Wilson et al. (Hum. Molec. Genet. 1: 353 only, 1992); this involved a single base change from G to A at position −308 in the promoter region of the gene. The less common allele, A at −308 (called T2), shows an increased frequency in patients with IDDM, but this depends on the concurrent increase in HLA-DR3 with which T2 is associated. A second TNF-alpha polymorphism, described by D'Alfonso and Richiardi (Immunogenetics 39: 150-154, 1994), also involves a G-to-A transition at position −238 of the gene. In alopecia greata, Galbraith and Pandey (Hum. Genet. 96: 433-436, 1995) found that the distribution of T1/T2 phenotypes differed between patients with the patchy form of the disease and patients with totalis/universalis disease. There was no significant difference in the distribution of the phenotypes for the second system. The results suggested genetic heterogeneity between the 2 forms of alopecia greata and suggested that the TNF-alpha gene is a closely linked locus within the major histocompatibility complex on chromosome 6 where this gene maps and may play a role in the pathogenesis of the patchy form of the disease.

Mulcahy et al. (Am. J. Hum. Genet. 59: 676-683, 1996) determined the inheritance of 5 microsatellite markers from the TNF region in 50 multiplex rheumatoid arthritis (RA) families. Overall, 47 different haplotypes were observed. One of these was present in 35.3% of affected, but in only 20.5% of unaffected, individuals (P less than 0.005). This haplotype accounted for 21.5% of the parental haplotypes transmitted to affected offspring and only 7.3% of the haplotypes not transmitted to affected offspring (P=0.0003). Further study suggested that the tumor necrosis factor—lymphotoxin (TNF-LT) region influences susceptibility to RA, distinct from HLA-DR. The study illustrated the use of the transmission disequilibrium test (TDT) as described by Spielman et al. (Am. J. Hum. Genet. 52: 506-516, 1993).

TNF-alpha may play a part in the pathogenesis of ankylosing spondylitis and rheumatoid arthritis. Gorman et al. (New Eng. J. Med. 346: 1349-1356, 2002) tested the efficacy of inhibition of TNF-alpha in treatment of ankylosing spondylitis. They used etanercept, a dimeric fusion protein of the human 75-kD (p75) TNFR2 (TNFRSF1B) linked to the Fc portion of human IgG1. Treatment in 40 patients with active, inflammatory disease for 4 months resulted in rapid, significant, and sustained improvement.

Ota et al. (Genes Immunity 1: 260-264, 2000) tested 192 sib pairs of adult Japanese women from 136 families for genetic linkage between osteoporosis and osteopenia phenotypes and allelic variants at the TNF-alpha locus, using a dinucleotide repeat polymorphism located near the gene. The TNF-alpha locus showed evidence for linkage to osteoporosis, with mean allele sharing of 0.478 (P=0.30) in discordant pairs and 0.637 (P=0.001) in concordant affected pairs. Linkage with osteopenia was also significant in concordant affected pairs (P=0.017). Analyses limited to the postmenopausal women in their cohort showed similar or even stronger linkage for both phenotypes.

Winchester et al. (Hum. Genet. 107: 591-596, 2000) studied the association of the −308G-A variant of the TNF-alpha gene and the insertion/deletion variant of angiotensin-converting enzyme (ACE) with a self-reported history of childhood asthma in 2 population groups. The −308A allele was significantly associated with self-reported childhood asthma in the UK/Irish population but not in the South Asian population. The ACE DD genotype was not associated with childhood asthma in either population. Thus, either the −308A allele or a linked major histocompatibility complex variant may be a genetic risk factor for childhood asthma in the UK/Irish sample.

Koss et al. (Genes Immun. 1: 185-190, 2000) found that women but not men with extensive compared to distal colitis were significantly more likely to bear the −308G-A promoter polymorphism of the TNF gene. The association was even stronger in women who also had an A rather than a C at position 720 in the LTA gene. These polymorphisms were also associated with significantly higher TNF production in patients with Crohn's disease, while an A instead of a G at position −238 in the TNF gene was associated with lower production of TNF in patients with ulcerative colitis.

Sashio et al. (Immunogenetics 53: 1020-1027, 2002) investigated the role of polymorphisms of the TNF gene and the TNFRSF1B gene in susceptibility to ulcerative colitis and Crohn's disease. They investigated 124 patients with Crohn's disease, 106 patients with ulcerative colitis, and 111 unrelated healthy controls. They examined 2 SNPs of the TNF-alpha gene: −308G-A and −238G-A. There was a difference in the carrier frequency for haplotype AG (−308A, −238G) between ulcerative colitis patients and the controls (odds ratio 4.76).

Van Heel et al. (Hum. Molec. Genet. 11: 1281-1289, 2002) stated that TNF expression is increased in inflammatory bowel disease (IBD) and that TNF maps to the IBD3 susceptibility locus. They showed by transmission disequilibrium and case-control analyses that in 2 independent Caucasian cohorts, a novel association of the TNF-857C promoter polymorphism with IBD was evident (overall P=0.001 in 587 IBD families). Further genetic associations of TNF-857C with IBD subphenotypes were seen for ulcerative colitis and for Crohn's disease, but only in patients not carrying common NOD2 mutations. These data suggested a recessive model of inheritance. The transcription factor OCT1 binds TNF-857T but not TNF-857C, and interacts in vitro and in vivo with the proinflammatory NFKB p65 subunit RELA at an adjacent binding site. The authors hypothesized that interaction of these transcription factors with specific alleles of TNF in gut tissue may be relevant to the pathogenesis of IBD.

In a case-control study of 304 Australian patients with Crohn's disease and 231 healthy controls, Fowler et al. (J. Med. Genet. 42: 523-528, 2005) found a significant association of the higher-producing IL10 −1082G and TNF-alpha −857C alleles with structuring disease. The association was strongest when these alleles were combined and persisted after multivariate analysis.

To investigate whether TNF-alpha promoter polymorphisms are associated with clearance of hepatitis B virus (HPV) infection, Kim et al. (Hum. Molec. Genet. 12: 2541-2546, 2003) genotyped 1,400 Korean subjects, 1,109 of whom were chronic HBV carriers and 291 who spontaneously recovered. The TNF promoter alleles that were previously reported to be associated with higher plasma levels (presence of −308A or the absence of −863A alleles), were strongly associated with the resolution of HBV infection. Haplotype analysis revealed that TNF-alpha haplotype 1 (−1103T; −863C; −857C; −308G; −238G; −163G) and haplotype 2 (−1031C; −863A; −857C; −308G; −238G; −163G) were significantly associated with HBV clearance, showing protective antibody production and persistent HBV infection, respectively (P=0.003-0.02).

In a first aspect, a compound is provided for use in the manufacture of a medicament for the treatment of a disease or disorder which is mediated by TNF-alpha activity, the compound having a structure:

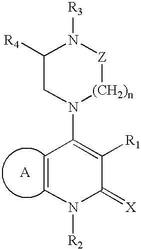

or a stereoisomer, or a pharmaceutically acceptable salt, ester, or solvate thereof, wherein A is a 5 to 7 membered ring having from 0 to 3 heteroatoms; R₁ is selected from the group consisting of —CN, —NO, —NO₂, —C(═O)R₁₂, —C(═O)OR₁₂, —C(═O)NR₁₀R₁₁, —NR₁₂C(═O)R₁₂, —SO₂NR₁₀R₁₁, —NR₁₂SO₂R₁₂, and —S(O)ₘR₁₂, wherein m is from 0 to 3; R₂ is selected from the group consisting of hydrogen, alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, heterocyclyl, substituted heterocyclyl, heterocyclylalkyl, substituted heterocyclylalkyl, heterocyclylaryl, substituted heterocyclylaryl, —(CH₂)ₓC(═O)aryl, substituted —(CH₂)ₓC(═O)aryl, —(CH₂)ₓC(═O)heterocyclyl, substituted —(CH₂)ₓC(═O)heterocyclyl, —(CH₂)ₓC(═O)heterocyclylalkyl, substituted —(CH₂)ₓC(═O)heterocyclylalkyl, —(CH₂)ₓC(═O)heterocyclylaryl, substituted —(CH₂)ₓC(═O)heterocyclylaryl, and —(CH₂)ₓNR₁₀R₁₁, wherein x is from 1 to 4; R₃ is selected from the group consisting of hydrogen, alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, heterocyclyl, substituted heterocyclyl, heterocyclylalkyl, substituted heterocyclylalkyl, heterocyclylaryl, substituted heterocyclylaryl, formyl, acetyl, and —(C═O)R₁₂; R₄ is selected from the group consisting of hydrogen, halogen, —R₁₂, —OR₁₂, —SR₁₂, and —NR₁₀R₁₁; R₁₀ and R₁₁ are independently selected from the group consisting of hydrogen, alkyl, alkenyl, cycloalkyl, aryl, alkylaryl, arylalkyl, acylalkyl, heterocyclyl, heterocyclylalkyl, and heterocyclylaryl, or R₁₀ and R₁₁ together with the nitrogen atom to which they are attached comprise a heterocycle or a substituted heterocycle; R₁₂ is independently selected from the group consisting of elected from the group consisting of hydrogen, alkyl, alkenyl, cycloalkyl, aryl, alkylaryl, arylalkyl, acylalkyl, heterocyclyl, heterocyclylalkyl, and heterocyclylaryl; X is selected from the group consisting of O and S; Z is selected from the group consisting of —C(═O)— and —CHR₁₂—; and n is 0, 1 or 2, with the proviso that when n is 0, Z is —C(═O)—.

In an embodiment of the first aspect, the compound of claim 1 has a formula:

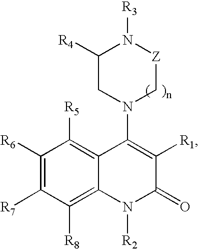

or a stereoisomer, or a pharmaceutically acceptable salt, ester, or solvate thereof, wherein R₁, R₂, R₃, R₄, Z, and n are as defined above; and wherein R₅, R₆, R₇, and R₈ are independently selected from the group consisting of hydrogen, halogen, —R₁₂, —OR₁₂, —SR₁₂, and —NR₁₀R¹¹.

In an embodiment of the first aspect, the compound of claim 1 has a formula selected from the group consisting of:

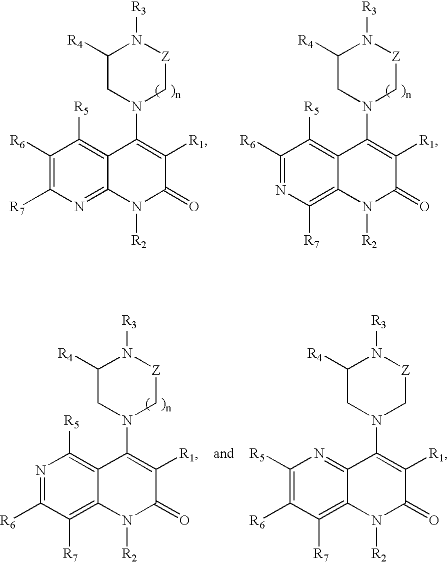

or a stereoisomer, or a pharmaceutically acceptable salt, ester, or solvate thereof, wherein R₁, R₂, R₃, R₄, Z, and n are as defined above; and wherein R₅, R₆, and R₇ are independently selected from the group consisting of hydrogen, halogen, —R₁₂, —OR₁₂, —SR₁₂, and —NR₁₀R₁₁.

In an embodiment of the first aspect, the compound of claim 1 has a formula selected from the group consisting of:

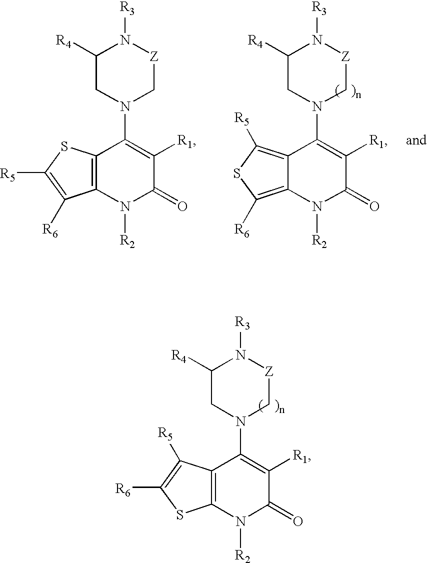

or a stereoisomer, or a pharmaceutically acceptable salt, ester, or solvate thereof, wherein R₁, R₂, R₃, R₄, Z, and n are as defined above; and wherein R₅ and R₆ are independently selected from the group consisting of hydrogen, halogen, —R₁₂, —OR₁₂, —SR₁₂, and —NR₁₀R₁₁.

In an embodiment of the first aspect, A is a 5 to 6 membered ring having a heteroatom selected from the group consisting of N and S; wherein R₁ is selected from the group consisting of —NO₂, —C(═O)R₁₂, —C(═O)OR₁₂, and —C(═O)NR₁₀R₁₁; and wherein R₂ is selected from the group consisting of R₂ is selected from the group consisting of alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, —(CH₂)ₓC(═O)aryl, and substituted —(CH₂)ₓC(═O)aryl.

In an embodiment of the first aspect, the compound is selected from the group consisting of:

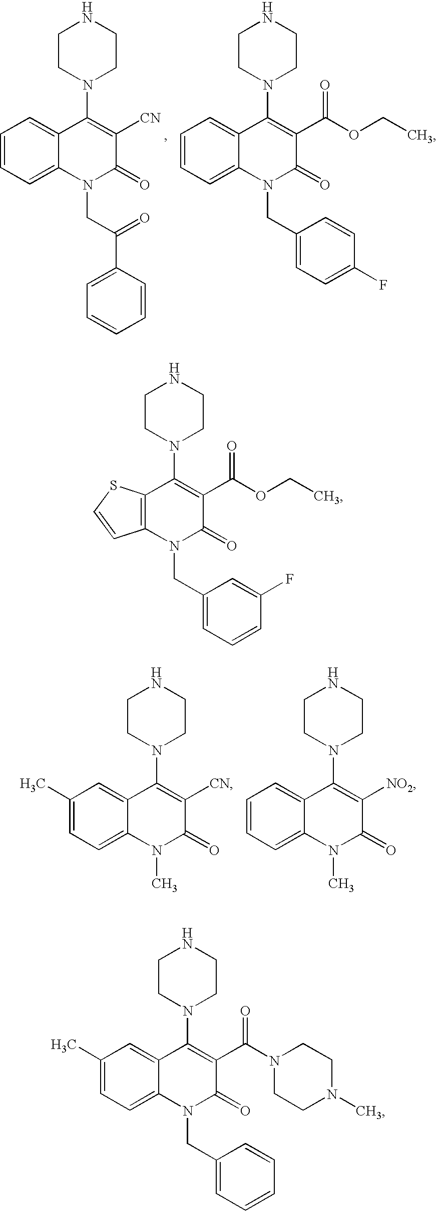

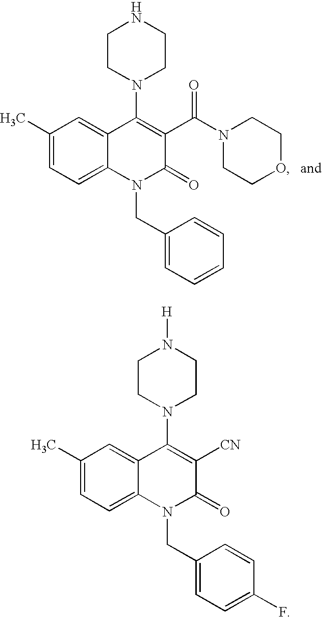

In an embodiment of the first aspect, the compound is in a form of a salt.

In an embodiment of the first aspect, the compound is for use as a pharmaceutical.

In an embodiment of the first aspect, a pharmaceutical composition is provided comprising a compound of the first aspect in association with at least one pharmaceutically acceptable excipient.

In a second aspect, a method of treating a disease or disorder which is mediated by TNF-alpha activity is provided, the method comprising administering to a subject in need of such treatment an effective amount of a compound of the first aspect.

In an embodiment of the second aspect, the disease or disorder is inflammation.

In an embodiment of the second aspect, the disease or disorder is septic shock.

In an embodiment of the second aspect, the disease or disorder is arthritis.

In an embodiment of the second aspect, the disease or disorder is cancer.

In an embodiment of the second aspect, the disease or disorder is acute respiratory distress syndrome.

In an embodiment of the second aspect, the disease or disorder is an inflammatory disease. The inflammatory disease can be selected from the group consisting of rheumatoid arthritis, osteoarthritis, inflammatory bowel disease, and asthma.

In an embodiment of the second aspect, the disease or disorder is an autoimmune disorder. The autoimmune disorder can be selected from the group consisting of diabetes, asthma, and multiple sclerosis.

In a third aspect, a method for suppressing an immune response in a subject in need thereof is provided, comprising administering an effective amount of the compound of the first aspect.

In a fourth aspect, a method for decreasing angiogenesis in a subject in need thereof is provided, comprising administering an effective amount of the compound of the compound of the first aspect.

In a fifth aspect, a method for treating a disease associated with excess glucocorticoid levels in a subject in need thereof is provided, comprising administering an effective amount of the compound of the first aspect.

In a sixth aspect, a method for treating a disease associated with excess glucocorticoid levels in a subject in need thereof is provided, comprising administering an effective amount of the compound of the compound of the first aspect.

In a seventh aspect, a method for treating a disease or disorder wherein TNF-alpha is pathogenic is provided, comprising administering to a subject in need thereof a compound of the compound of the first aspect and a drug for treating the disease or disorder, wherein the drug has no measurable TNF-alpha inhibiting activity.

In an eighth aspect, a method for treating inflammation or septic shock is provided, comprising administering to a subject in need thereof a compound of the first aspect and a steroid.

In a ninth aspect, a method for treating rheumatoid arthritis is provided, comprising administering to a subject in need thereof a compound of the first aspect and a steroid.

In a tenth aspect, a method for treating asthma or acute respiratory distress is provided, comprising administering to a subject in need thereof a compound of the first aspect and a corticosteroid. The corticosteroid can be selected from the group consisting of cortisone, hydrocortisone, methylprednisolone, prednisone, prednisolone, betamethasone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

In an eleventh aspect, a method for treating asthma or acute respiratory distress is provided, comprising administering to a subject in need thereof a compound of the first aspect and a drug selected from the group consisting of beclomethasone, fluticasone, triamcinolone, mometasone, prednisone, prednisolone, methylprednisolone, an azatadine, carbinoxamine/pseudoephedrine, cetirizine, cyproheptadine, dexchlorpheniramine, fexofenadine, loratadine, promethazine, tripelennamine, brompheniramine, cholopheniramine, clemastine, diphenhydramine, and epinephrine.

In a twelfth aspect, a method for treating irritable bowel disease is provided, comprising administering to a subject in need thereof a compound of the first aspect and azathioprine or a corticosteroid.

In a thirteenth aspect, a method for treating cancer is provided, comprising administering to a subject in need thereof a compound of the first aspect and paclitaxel.

In a fourteenth aspect, a method for treating an immune disorder is provided, comprising administering to a subject in need thereof a compound of the first aspect and an immunosuppressive compound.

In a fifteenth aspect, a method for treating an immune disorder is provided, comprising administering to a subject in need thereof a compound of the first aspect and an immunosuppressive compound, wherein the immune disorder is Lyme disease, Lupus, or Acquired Immune Deficiency Syndrome.

In a sixteenth aspect, a method for treating an immune disorder is provided, comprising administering to a subject in need thereof a compound of the first aspect and a drug selected from the group consisting of a protease inhibitor, a nucleoside reverse transcriptase inhibitor, a nucleotide reverse transcriptase inhibitor, a non-nucleoside reverse transcriptase inhibitor, a biological response modifier, a compound that inhibits or interferes with tumor necrosing factor, and an antiviral.

In a seventeenth aspect, a method for treating an immune disorder is provided, comprising administering to a subject in need thereof a compound of the first aspect and a drug selected from the group consisting of indinavir, amprenavir, saquinavir, lopinavir, ritonavir, nelfinavir zidovudine, abacavir, lamivudine, idanosine, zalcitabine, stavudine, tenofovir disoproxil fumarate delavirdine, efavirenz, nevirapine, etanercept, infliximab, amivudine, and zidovudine.

In an eighteenth aspect, use is provided of a compound of the first aspect and a drug selected from the group consisting of a nonsteroidal anti-inflammatory drug, an anti-infective drug, a beta stimulant, a steroid, an antihistamine, an anticancer drug, an asthma drug, a sepsis drug, an arthritis drug, and an immunosuppressive drug in the preparation of a pharmaceutical composition for treating a disease or disorder wherein TNF-alpha is pathogenic.

In a nineteenth aspect, use is provided of a compound of the first aspect and a beta stimulant selected from the group consisting of a bronchodilator, an inhalation corticosteroid, and a hormone in the preparation of a pharmaceutical composition for treating a disease or disorder wherein TNF-alpha is pathogenic.

In a twentieth aspect, use is provided of a compound of the first aspect and an inhalation corticosteroid selected from the group consisting of beclomethasone, fluticasone, triamcinolone, mometasone, prednisone, prednisolone, and methylprednisolone in the preparation of a pharmaceutical composition for treating a disease or disorder wherein TNF-alpha is pathogenic.

In a twenty-first aspect, use is provided of a compound of the first aspect and an antihistamine selected from the group consisting of azatadine, carbinoxamine/pseudoephedrine, cetirizine, cyproheptadine, dexchlorpheniramine, fexofenadine, loratadine, promethazine, tripelennamine, brompheniramine, cholopheniramine, clemastine, diphenhydramine, and epinephrine in the preparation of a pharmaceutical composition for treating a disease or disorder wherein TNF-alpha is pathogenic.

In a twenty-second aspect, use is provided of a compound of the first aspect and a steroid selected from the group consisting of cortisone, hydrocortisone, methylprednisolone, prednisone, prednisolone, betamethasone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone in the preparation of a pharmaceutical composition for treating a disease or disorder wherein TNF-alpha is pathogenic.

In a twenty-third aspect, use is provided of a compound of the first aspect and an anti-infective drug selected from the group consisting of an anthelmintic, an aminoclycoside, an antifungal antibiotic, a cephalosporin, a beta-lactam antibiotic, chloramphenicol, a macrolide, a penicillin, a tetracycline, bacitracin, clindamycin, colistimethate sodium, polymyxin b sulfate, vancomycin, antivirals, acyclovir, amantadine, didanosine, efavirenz, foscamet, ganciclovir, indinavir, lamivudine, nelfinavir, ritonavir, saquinavir, stavudine, valacyclovir, valganciclovir, zidovudine, a quinolone, a sulfonamide, furazolidone, metronidazole, pentamidine, sulfanilamidum crystallinum, gatifloxacin, and sulfamethoxazole/trimethoprim in the preparation of a pharmaceutical composition for treating a disease or disorder wherein TNF-alpha is pathogenic.

In a twenty-fourth aspect, use is provided of a compound of the first aspect and an anti-infective drug selected from the group consisting of mebendazole, gentamicin, neomycin, tobramycin, amphotericin b, fluconazole, griseofulvin, itraconazole, ketoconazole, nystatin, micatin, tolnaftate, cefaclor, cefazolin, cefotaxime, ceftazidime, ceftriaxone, cefuroxime, cephalexin, cefotetan, meropenem, azithromycin, clarithromycin, erythromycin, penicillin G sodium salt, amoxicillin, ampicillin, dicloxacillin, nafcillin, piperacillin, ticarcillin, doxycycline, minocycline, tetracycline, ciprofloxacin, levofloxacin, sulfadiazine, sulfisoxazole, and dapsone in the preparation of a pharmaceutical composition for treating a disease or disorder wherein TNF-alpha is pathogenic.

In a twenty-fifth aspect, use is provided of a compound of the first aspect and a nonsteroidal anti-inflammatory drug selected from the group consisting of celecoxib, rofecoxib, aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin in the preparation of a pharmaceutical composition for treating a disease or disorder wherein TNF-alpha is pathogenic.

In a twenty-sixth aspect, use is provided of a compound of the first aspect in a pharmaceutically acceptable carrier in the preparation of a pharmaceutical composition for treating a disease or disorder wherein TNF-alpha is pathogenic.

Accordingly, the TNF-alpha inhibitors of preferred embodiments are useful in the treatment of the above-referenced diseases and disorders. These and other embodiments and aspects thereof will be apparent upon reference to the following detailed description. To this end, various references are set forth herein which describe in more detail certain procedures, compounds and/or compositions, and are hereby incorporated by reference in their entirety.

### BRIEF DESCRIPTION OF THE DRAWINGS

FIG. 1 provides an +ES selected ion chromatogram at MH⁺ of VAQ996 and metabolite ml after incubation of NVP-VAQ996-NX-1 (5 μM) with liver microsomes of mouse (0.1 mg microsomal protein/mL).

FIG. 2 provides an +ES selected ion chromatogram at MH⁺ of VAQ996 and metabolite ml after incubation of NVP-VAQ996-NX-1 (5 μM) with liver microsomes of human (0.1 mg microsomal protein/mL).

FIG. 3 provides an +ES selected ion chromatogram at MH⁺ of VAQ996 and metabolite ml after incubation of NVP-VAQ996-NX-1 (5 μM) in plasma of mice.

FIG. 4 provides an +ES selected ion chromatogram at MH⁺ of VAQ996 and metabolite ml in plasma 1 h after p.o. administration of 10 mg/kg NVP-VAQ996-NX-1 to mice.

FIG. 5 provides an +ES product ion spectrum of NVP-VAQ996-NX (MH⁺=m/z 483).

FIG. 6 provides an +ES product ion spectrum of ml (MH⁺=m/z 373).

FIG. 7 provides an +ES product ion spectrum of NVP-VAR235—NX (MH⁺=m/z 373).

### DETAILED DESCRIPTION OF THE PREFERRED EMBODIMENT

The following description and examples illustrate a preferred embodiment of the present invention in detail. Those of skill in the art will recognize that there are numerous variations and modifications of this invention that are encompassed by its scope. Accordingly, the description of a preferred embodiment should not be deemed to limit the scope of the present invention.

As an aid to understanding the preferred embodiments, certain definitions are provided herein.

The term “TNF-alpha activity” as used herein is a broad term, and is to be given its ordinary and customary meaning to a person of ordinary skill in the art (and is not to be limited to a special or customized meaning), and refers without limitation to an activity or effect mediated at least in part by tumor necrosis factor alpha.

The term “inhibitor” as used herein is a broad term, and is to be given its ordinary and customary meaning to a person of ordinary skill in the art (and is not to be limited to a special or customized meaning), and refers without limitation to a molecule (e.g., natural or synthetic compound) that can decrease at least one activity of TNF-alpha. In other words, an “inhibitor” alters activity if there is a statistically significant change in the amount of TNF-alpha measured, in TNF-alpha activity, or in TNF-alpha detected extracellularly and/or intracellularly in an assay performed with an inhibitor, compared to the assay performed without the inhibitor.

In general, TNF-alpha inhibitors inhibit the physiological function of TNF-alpha, and thus are useful in the treatment of diseases where TNF-alpha may be pathogenic.

A preferred class of TNF-alpha inhibitors includes compounds of the formula:

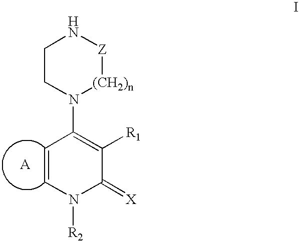

wherein Z is CH₂ or C═O; X is O or S; ring A together with the N-containing heterocycle to which it is attached is a heterocyclyl, such as aromatic heterocyclyl, e.g., a fused ring heterocyclyl, preferably comprising 10, 11, or 12 ring members and 1, 2, 3, or 4 heteroatoms including the nitrogen atom (preferably 1 or 2 heteroatoms), selected from N, O, and S (preferably N and/or S), with the proviso that at least one nitrogen heteroatom is present in A together with the N-containing heterocycle to which it is attached, preferably quinolinyl, naphthyridinyl or thienopyridinyl; R₁ is —NO, —NO₂, or the residue of a sulfonic or carbonic acid, such as —CN, —C(═O)R₆, —C(═O)OR₆, —C(═O)NR₅, —NR₆C(═O)R₆, —SO₂NR₄R₅, —NR₆SO₂R₆, or —S(O)ₘR₆; R₂ is hydrogen, alkyl, alkenyl, arylalkyl, acylalkyl, cycloalkyl, aryl, alkylaryl, heterocyclyl, heterocyclylalkyl, heterocyclylaryl, —(CH₂)ₓNR₅, —(CH₂)ₓC(═O)aryl, or —(CH₂)ₓC(═O)heterocyclyl; R₄ and R₅ are independently hydrogen, alkyl, acylalkyl, alkenyl, cycloalkyl, aryl, arylalkyl, heterocyclyl or heterocycloalkyl, or R₄ and R₅ together with the nitrogen atom to which they are attached are heterocyclyl; R₆ is hydrogen, alkyl, arylalkyl, acylalkyl, cycloalkyl, aryl, heterocyclylalkyl, alkenyl, cycloalkyl, aryl, alkylaryl, heterocyclyl or alkylheterocyclyl; m is 0, 1 or 2; n is 0, 1 or 2; and x is 1 to 4.

Each group (or substituent) defined herein preferably includes from 1 to 18 carbon atoms; however, in certain embodiments the group or substituent can include more than 18 carbon atoms. Especially preferred are alkyl including (C₁₋₁₂) alkyl; alkenyl including (C₂₋₁₂)alkenyl; alkoxy including (C₁₋₁₂)alkoxy; alkylthio including (C₁₋₁₂)alkylthio; acyl including (C₂₋₁₂)acyl; cycloalkyl including (C₃₋₁₂)cycloalkyl; aryl including (C₆₋₁₂)aryl; and heterocyclyl including aliphatic heterocyclyl and aromatic heterocyclyl having 3 to 12 ring members and 1 to 4 heteroatoms preferably selected from N, O, and S.

Alkyl, alkenyl, arylalkyl, acylalkyl, heterocycloalkyl, cycloalkyl, aryl, alkylaryl, heterocyclylaryl, heterocyclyl, alkylheterocycyl can be unsubstituted or substituted, e.g., unsubstituted or substituted by halogen, —CN, —NO, —NO₂, —CF₃, —OCF₃, alkoxy, alkylthio, or other substituents as disclosed herein. Ring A preferably includes an unsubstituted ring A or ring A substituted by 1 or more (e.g., 2 or more) substituents selected from halogen, —CN, —NO, —NO₂, —CF₃, —OCF₃, —NHSO₂R₆, —COR₆, —COOR₆, —OCOR₆, —CONR₄R₅, —NR₄COR₆, —SO₂NR₅, —OR₆, —S(O)_{y}R₆, —SR₆, —COOH, —NHCOR₆, —(CH₂)_{y}COaryl, —(CH₂ NR₄R₅, wherein R₄, R₅, R₆ are as defined above and y is 0, 1, 2, 3 or 4.

Another preferred class of TNF-alpha inhibitors includes compounds of the formula:

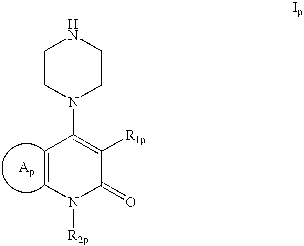

wherein Aₚ together with the N-containing heterocycle to which it is attached is preferably an aromatic heterocyclyl having 11 to 12 ring members and 2 hetero atoms selected from N and S, with the proviso that at least one nitrogen heteroatom is present, and wherein heterocyclyl is as defined above, but preferably unsubstituted or substituted with (C₁₋₄)alkyl, and preferably quinolinyl, naphthyridinyl or thienopyridinyl, more preferably, quinolinyl or thienopyridinyl, such as a quinolinyl or thieno[2,3-b]pyridinyl, for example, an unsubstituted quinolinyl or quinolinyl substituted by (C₁₋₄)alkyl (e.g., methyl), or unsubstituted thienopyridinyl. R₁ₚ is preferably —NO₂, or the residue of a carbonic acid or carboxylic acid attached to the ring via the carbon atom of the carbonyl or nitrile function, for example, an ester group, an amide group, or a nitrile group. R₂ₚ is preferably substituted or unsubstituted alkyl, e.g., (C₁₋₄)alkyl unsubstituted or substituted by (C₆₋₁₈)aryl, or (C₆₋₁₈)arylcarbonyl (e.g., phenyl or phenylcarbonyl, e.g., wherein (C₆₋₁₈)aryl is unsubstituted or substituted, e.g., by one or more halogen). The residue of a carbonic acid attached via the carbonyl or nitrile functional carbon atom can include, e.g., an ester group, an amide group, or a nitrile group, for example, —NO₂, —CN, —C(O)OR₃, or —C(O)NR₄R₅; wherein R₃ is alkyl, such as (C₁₋₆)alkyl, (C₆₋₁₈)aryl, or (C₆₋₈)aryl(C₁₋₄)alkyl, preferably as (C₁₋₄)alkyl; R₄ and R₅ independently of each other are (C₁₋₈)alkyl, or R₄ and R₅ together with the nitrogen atom to which they are attached are heterocyclyl, such as aliphatic heterocyclyl having 4, 5, 6, or 7 ring members and having 1, 2, 3, or 4 heteroatoms, e.g., selected from N, O, and S, preferably N and O, wherein heterocyclyl is unsubstituted or substituted, e.g., unsubstituted or substituted by alkyl, such as (C₁₋₄)alkyl, preferably R₄ and R₅ together with the nitrogen atom to which they are attached are heterocyclyl.

In preferred embodiments, the compound of formula Iₚ is one wherein ring Aₚ together with the N-containing heterocycle to which it is attached is quinolinyl, e.g., unsubstituted quinolinyl or quinolinyl substituted by (C₁₋₄)alkyl, e.g., in position 6 of the ring system; or thienopyridinyl, such as thieno[2,3-b]pyridinyl. R₁ₚ is preferably —NO₂, —CN, —C(O)OR₃ₚ, or —C(O)N₄ₚR₅ₚ, wherein R₃ₚ is preferably (C₁₋₆)alkyl, R₄ₚ and R₅ₚ together with the nitrogen atom to which they are attached are preferably heterocyclyl, such as aliphatic heterocyclyl, having 4, 5, 6, or 7 ring members and having 1, 2, 3, or 4 heteroatoms selected from N, O, or S (preferably N or O), e.g., morpholinyl or piperazinyl, e.g., unsubstituted heterocyclyl or heterocyclyl substituted by (C₁₋₄)alkyl, such as morpholinyl or piperazinyl substituted by (C₁₋₄)alkyl, e.g., 4-methyl-piperazin-1-yl. R₂ₚ is preferably alkyl, e.g., (C₁₋₄)alkyl (e.g., methyl), or unsubstituted alkyl or substituted alkyl, e.g., unsubstituted alkyl or alkyl substituted by phenyl or phenylcarbonyl, e.g., wherein phenyl is unsubstituted or substituted, e.g., unsubstituted or substituted by one or more substituents, e.g. halogen.

In the compounds of formula I and of formula Iₚ, each single defined substituent can be a preferred substituent, e.g., independently of each other substituent defined.

In preferred embodiments, the compounds of formula I and/or formula Iₚ are selected from the group consisting of:

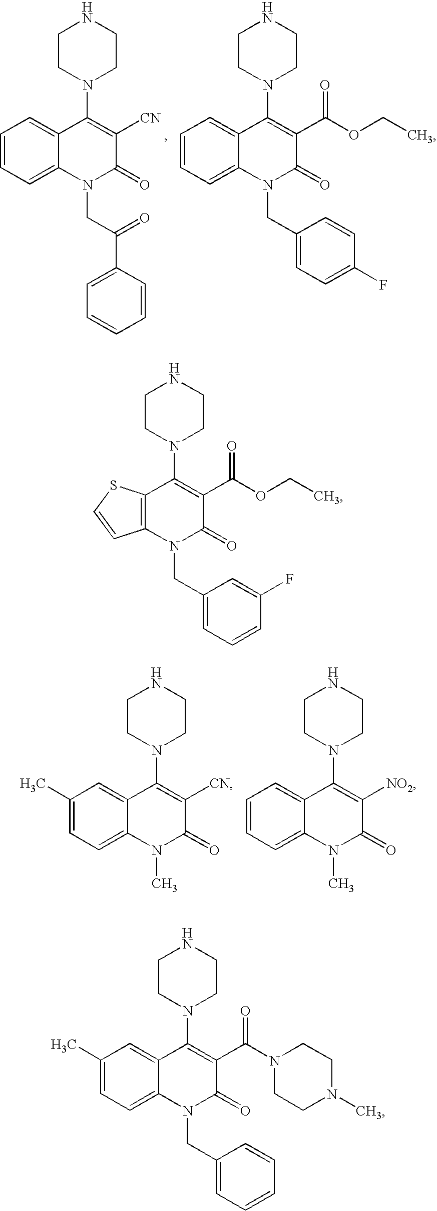

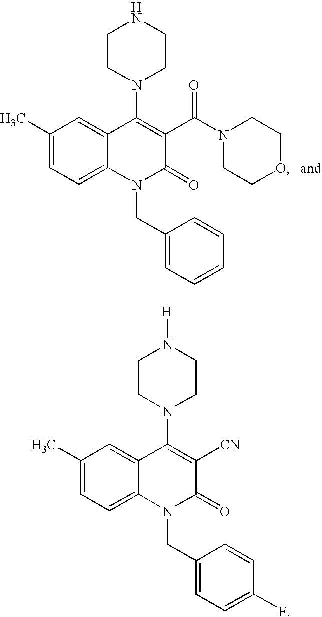

In certain of the preferred embodiments, inhibitors of TNF-alpha are provided that have the following structures:

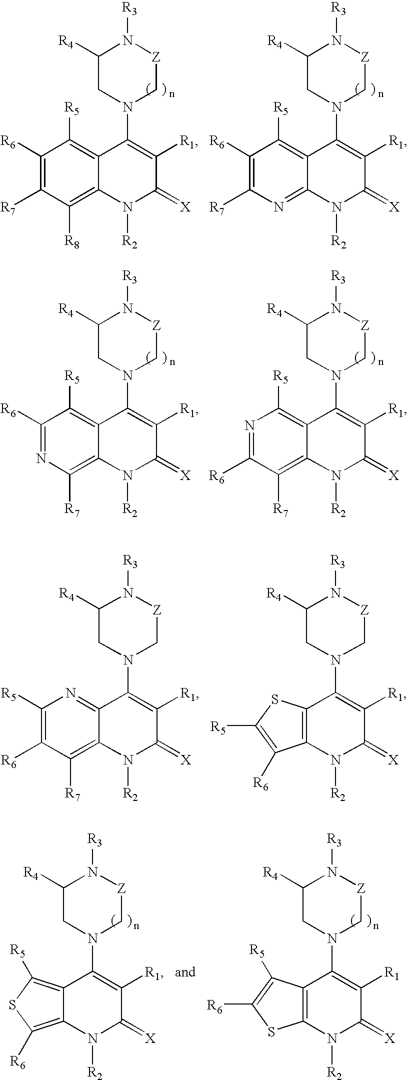

including stereoisomers, prodrugs and pharmaceutically acceptable salts and esters thereof, wherein: X is O or S; Z is —CH₂—, —C(═O)— or CHR₉; n is 0, 1 or 2, with the proviso that when n is 0, Z is —C(═O)—; R₁ is selected from the group consisting of hydrogen, alkyl, substituted alkyl, cycloalkyl, substituted cycloalkyl, aryl, substituted aryl, arylalkyl, substituted arylalkyl, heterocycle, substituted heterocycle, —(CH₂)ₓC(═O)aryl, —(CH₂)ₓC(═O) substituted aryl, —(CH₂)ₓC(═O)heterocycle, —(CH₂)ₓC(═O) substituted heterocycle, and —(CH₂)ₓNR₁₀R₁₁, wherein x is 1 to 4; R₁₀ and R₁₁ are independently selected from the group consisting of hydrogen, alkyl, substituted alkyl, aryl, substituted aryl, arylalkyl, substituted arylalkyl, heterocycle, substituted heterocycle, heterocycloalkyl, and substituted heterocyclealkyl, or R₁₀ and R₁₁ taken together comprise a heterocycle or a substituted heterocycle. R₂ is selected from the group consisting of —CN, —NO, —NO₂, —C(═O)R₁₂, —C(═O)OR₁₂, —C(═O)NR₁₀R₁₁, —NR₁₂C(═O)R₁₂, —SO₂NR₁₀R₁₁, NR₁₂SO₂R₁₂, and —S(O)ₘR₁₂; and m is 0, 1, 2, or 3. R₁₂ is selected from the group consisting of alkyl, substituted alkyl aryl, substituted aryl, arylalkyl, substituted arylalkyl, heterocycle, and substituted heterocycle. R₃, R₄, R₅, R₆, R₈ and R₉ are the same or different and are independently, hydrogen, halogen, —R₁₃, —OR₁₃, —SR₁₃ or —NR₁₃R₁₄. R₁₃ and R₁₄ are the same or different and are independently hydrogen, alkyl, substituted alkyl, aryl, methylene-oxy-aryl, substituted aryl, arylalkyl, substituted arylalkyl, heterocycle, substituted heterocycle, heterocyclealkyl or substituted heterocyclealkyl; or R₁₃ and R₁₄ taken together with the nitrogen atom to which they are attached form a heterocycle or substituted heterocycle; R₇ is hydrogen, alkyl, substituted alkyl, formyl, acetyl, —(C═O)R₁₃, cycloalkyl, substituted cycloalkyl, aryl, substituted aryl, arylalkyl, substituted arylalkyl, heterocycle, or substituted heterocycle.

In a preferred embodiment, methods are provided for reducing TNF-alpha activity in a patient in need thereof by administering to the patient an effective amount of the above-described compounds.

The term “alkyl” as used herein is a broad term, and is to be given its ordinary and customary meaning to a person of ordinary skill in the art (and is not to be limited to a special or customized meaning), and refers without limitation to a straight chain or branched, acyclic or cyclic, unsaturated or saturated aliphatic hydrocarbon containing 1, 2, 3, 4, 5, 6, 7, 8, 9, 10, 11 or 12 or more carbon atoms, while the term “lower alkyl” has the same meaning as alkyl but contains 1, 2, 3, 4, 5, or 6 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like.

The term “cycloalkyl” as used herein is a broad term, and is to be given its ordinary and customary meaning to a person of ordinary skill in the art (and is not to be limited to a special or customized meaning), and refers without limitation to alkyls that include mono-, di-, or poly-homocyclic rings. Cycloalkyls are also referred to as “cyclic alkyls” or “homocyclic rings.” Representative saturated cyclic alkyls include cyclopropyl, cyclobutyl, cyclopentyl, cyclohexyl, —CH₂cyclopropyl, —CH₂cyclobutyl, —CH₂cyclopentyl, —CH₂cyclohexyl, and the like; while unsaturated cyclic alkyls include cyclopentenyl and cyclohexenyl, and the like. Cyclic alkyls include decalin, adamantane, and the like.

The term “aryl” as used herein is a broad term, and is to be given its ordinary and customary meaning to a person of ordinary skill in the art (and is not to be limited to a special or customized meaning), and refers without limitation to an aromatic carbocyclic moiety such as phenyl or naphthyl.

The term “arylalkyl” as used herein is a broad term, and is to be given its ordinary and customary meaning to a person of ordinary skill in the art (and is not to be limited to a special or customized meaning), and refers without limitation to an alkyl having at least one alkyl hydrogen atom replaced with an aryl moiety, such as benzyl, —CH₂(1-naphthyl), —CH₂(2-naphthyl), —(CH₂)₂phenyl, —(CH₂)₃phenyl, —CH(phenyl)₂, and the like.

The term “heteroaryl” as used herein is a broad term, and is to be given its ordinary and customary meaning to a person of ordinary skill in the art (and is not to be limited to a special or customized meaning), and refers without limitation to an aromatic heterocycle ring of 5 or 6 to 7, 8, 9, 10, 11, or 12 members and having at least one heteroatom (or 2, 3, or 4 or more heteroatoms) selected from nitrogen, oxygen, and sulfur, and containing at least one carbon atom, including both monocyclic and bicyclic ring systems. Representative heteroaryls include (but are not limited to) furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

The term “heteroarylalkyl” as used herein is a broad term, and is to be given its ordinary and customary meaning to a person of ordinary skill in the art (and is not to be limited to a special or customized meaning), and refers without limitation to an alkyl having at least one alkyl hydrogen atom replaced with a heteroaryl moiety, such as —CH₂pyridinyl, —CH₂pyrimidinyl, and the like.

The terms “heterocycle” and “heterocycle ring” as used herein are broad terms, and are to be given their ordinary and customary meaning to a person of ordinary skill in the art (and are not to be limited to special or customized meanings), and refer without limitation to a 5, 6, or, 7 membered monocyclic heterocyclic ring, or a 7, 8, 9, 10, 11, 12, 13, or 14 or more membered polycyclic heterocyclic ring. The ring can be saturated, unsaturated, aromatic, or nonaromatic, and can contain 1, 2, 3, or 4 or more heteroatoms independently selected from nitrogen, oxygen, and sulfur. The nitrogen and sulfur heteroatoms may be optionally oxidized, and the nitrogen heteroatom may be optionally quaternized, including bicyclic rings in which any of the above heterocycles are fused to a benzene ring as well as tricyclic (and higher) heterocyclic rings. The heterocycle can be attached via any heteroatom or carbon atom of the ring or rings. Heterocycles include heteroaryls as defined above. Thus, in addition to the aromatic heteroaryls listed above, heterocycles also include (but are not limited to) morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydropyrimidinyl, tetrahydrothiophenyl, tetrahydrothiopyranyl, tetrahydropyrimidinyl, tetrahydrothiophenyl, tetrahydrothiopyranyl, and the like.

The term “eterocyclealkyl” as used herein is a broad term, and is to be given its ordinary and customary meaning to a person of ordinary skill in the art (and is not to be limited to a special or customized meaning), and refers without limitation to an alkyl having at least one alkyl hydrogen atom replaced with a heterocycle, such as —CH₂-morpholinyl, and the like.

The term “substituted” as used herein is a broad term, and is to be given its ordinary and customary meaning to a person of ordinary skill in the art (and is not to be limited to a special or customized meaning), and refers without limitation to any of the above groups (e.g., alkyl, aryl, arylalkyl, heteroaryl, heteroarylalkyl, heterocycle or heterocyclealkyl) wherein at least one hydrogen atom is replaced with a substituent. In the case of a keto substituent (i.e., —C(═O)—) two hydrogen atoms are replaced. When substituted, “substituents,” within the context of preferred embodiments, include halogen, hydroxy, cyano, nitro, amino, alkylamino, dialkylamino, alkyl, alkoxy, alkylthio, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NR_{b}R_{c}, —NRₐC(═O)OR_{b}, —NRₐSO₂R_{b}, —ORₐ, —C(═O)Rₐ, —C(═O)ORₐ, —C(═O)NRₐR_{b}, —OC(═O)NRₐR_{b}, —SH, —SRₐ, —SORA, —S(═O)₂Rₐ, —OS(═O)₂Rₐ, —S(═O)₂ORₐ, wherein Rₐ, R_{b}, and R_{c} are the same or different and are independently selected from hydrogen, alkyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

The term “halogen” as used herein is a broad term, and is to be given its ordinary and customary meaning to a person of ordinary skill in the art (and is not to be limited to a special or customized meaning), and refers without limitation to fluoro, chloro, bromo, and iodo.

The term “haloalkyl” as used herein is a broad term, and is to be given its ordinary and customary meaning to a person of ordinary skill in the art (and is not to be limited to a special or customized meaning), and refers without limitation to an alkyl having at least one hydrogen atom replaced with halogen, such as trifluoromethyl and the like.

The term “alkoxy” as used herein is a broad term, and is to be given its ordinary and customary meaning to a person of ordinary skill in the art (and is not to be limited to a special or customized meaning), and refers without limitation to an alkyl moiety attached through an oxygen bridge (i.e., —O-alkyl) such as methoxy, ethoxy, and the like.

The term “thioalkyl” as used herein is a broad term, and is to be given its ordinary and customary meaning to a person of ordinary skill in the art (and is not to be limited to a special or customized meaning), and refers without limitation to an alkyl moiety attached through a sulfur bridge (i.e., —S-alkyl) such as methylthio, ethylthio, and the like.

The term “alkylsulfonyl” as used herein is a broad term, and is to be given its ordinary and customary meaning to a person of ordinary skill in the art (and is not to be limited to a special or customized meaning), and refers without limitation to an alkyl moiety attached through a sulfonyl bridge (i.e., —SO₂-alkyl) such as methylsulfonyl, ethylsulfonyl, and the like.

The terms “alkylamino” and “dialkylamino” as used herein are broad terms, and are to be given their ordinary and customary meanings to a person of ordinary skill in the art (and are not to be limited to special or customized meanings), and refer without limitation to one alkyl moiety or two alkyl moieties, respectively, attached through a nitrogen bridge (i.e., —N-alkyl) such as methylamino, ethylamino, dimethylamino, diethylamino, and the like.

The term “hydro alkyl” as used herein is a broad term, and is to be given its ordinary and customary meaning to a person of ordinary skill in the art (and is not to be limited to a special or customized meaning), and refers without limitation to an alkyl substituted with at least one hydroxyl group.

The term “mono- or di-(cycloalkyl)methyl” as used herein is a broad term, and is to be given its ordinary and customary meaning to a person of ordinary skill in the art (and is not to be limited to a special or customized meaning), and refers without limitation to a methyl group substituted with one or two cycloalkyl groups, such as cyclopropylmethyl, dicyclopropylmethyl, and the like.

The term “alkylcarbonylalkyl” as used herein is a broad term, and is to be given its ordinary and customary meaning to a person of ordinary skill in the art (and is not to be limited to a special or customized meaning), and refers without limitation to an alkyl substituted with a —C(═O)-alkyl group.

The term “alkylcarbonyloxyalkyl” as used herein is a broad term, and is to be given its ordinary and customary meaning to a person of ordinary skill in the art (and is not to be limited to a special or customized meaning), and refers without limitation to an alkyl substituted with a —C(═O)O-alkyl group or a —OC(═O)-alkyl group.

The term “alkylthioalkyl” as used herein is a broad term, and is to be given its ordinary and customary meaning to a person of ordinary skill in the art (and is not to be limited to a special or customized meaning), and refers without limitation to an alkyl substituted with an —O-alkyl group.

The term “alkylthioalkyl” as used herein is a broad term, and is to be given its ordinary and customary meaning to a person of ordinary skill in the art (and is not to be limited to a special or customized meaning), and refers without limitation to an alkyl substituted with a —S-alkyl group.

The term “mono- or di-(alkyl)amino” as used herein is a broad term, and is to be given its ordinary and customary meaning to a person of ordinary skill in the art (and is not to be limited to a special or customized meaning), and refers without limitation to an amino substituted with one alkyl or with two alkyls, respectively.

The term “mono- or di-(alkyl)aminoalkyl” as used herein is a broad term, and is to be given its ordinary and customary meaning to a person of ordinary skill in the art (and is not to be limited to a special or customized meaning), and refers without limitation to an alkyl substituted with a mono- or di-(alkyl)amino.

The cyclic systems referred to herein include fused ring, bridged ring, and spiro ring moieties, in addition to isolated monocyclic moieties.

### Applications and Methods Utilizing TNF-Alpha Inhibitors

TNF-alpha inhibitors have a variety of applicable uses, as noted above. Candidate TNF-alpha inhibitors may be isolated or procured from a variety of sources, such as bacteria, fungi, plants, parasites, libraries of chemicals (small molecules), peptides or peptide derivatives and the like. Further, one of skill in the art will recognize that inhibition has occurred when a statistically significant variation from control levels is observed.

The compounds of the preferred embodiments exhibit pharmacological activity and are therefore useful as pharmaceuticals. E.g., compounds of formula I and other compounds of preferred embodiments are found to interfere with TNF-alpha activity by inhibition of TNF-alpha production in LPS-challenged mice, e.g., compounds of the preferred embodiments can inhibit TNF-alpha production significantly. Compounds of the preferred embodiments also show activity in the FITC-induced DTH model in mice, e.g., thus showing anti-inflammatory activity.

Pharmaceutical compositions of the preferred embodiments can be used for treating disorders which are mediated by TNF-alpha activity. Methods of treating disorders which are mediated by TNF-alpha activity, which treatment comprises administering to a subject in need of such treatment an effective amount of a compound of the preferred embodiment, e.g., in the form of a pharmaceutical composition, are also provided. Also provided are compounds of the preferred embodiments for the manufacture of a medicament, and the use of a compound of the preferred embodiments for the manufacture of a medicament, e.g., a pharmaceutical composition, for the treatment of disorders, which are mediated by TNF-alpha activity. Treatment includes treatment of an existing disease or disorder, as well as prophylaxis (prevention) of a disease or disorder.

Disorders, e.g., including diseases, which can be treated with compounds of the preferred embodiments, e.g., by inhibition or suppression of TNF-alpha activity, include those which are mediated by TNF-alpha activity. Such disorders (diseases) include (chronic) inflammatory diseases, allergic diseases, autoimmune diseases, cardiovascular diseases, neurodegenerative diseases, viral diseases, such as retroviral diseases, cancer, pain, diseases following transplantation. More specifically, the disorders or diseases include rheumatoid arthritis, osteoarthritis, osteoporosis, multiple sclerosis, atherosclerosis, psoriasis, systemic lupus erythomatodes (SLE), (acute) glomerulonephritis, asthma, such as asthma bronchiale, chronic obstructive pulmonary diseases (COPD), respiratory distress-syndrome (ARDS), inflammatory bowel disease (e.g., Crohn's Disease), colitis (e.g., ulcerative colitis), sepsis, malaria (e.g., cerebral form of malaria), AIDS, neurodegenerative diseases such as Alzheimer's disease, Parkinson's disease, Guillain-Barre-syndrome, graft-versus-host-disease (GvHD), vasculitis, uveitis, (insulin-dependent) diabetes (e.g., diabetes mellitus), (adult) consequences of (multiple) trauma (e.g., organ dysfunction), acute and chronic pain (e.g., neuropathic pain, such as primary and secondary hyperalgesia, dynamic and static allodynia), stroke, cardiac infarction, dermatitis (e.g., inflammatory dermatitis), atopic dermatitis, alopecia, rhinitis (allergica), allergic conjunctivitis, meningitis, myasthenia gravis, sclerodermitis, sarcoidosis; and cancer, such as cancerous forms of the blood building system.

The preferred embodiments provide one or more compounds of the preferred embodiments for use as a pharmaceutical; or the use of one or more compounds of the preferred embodiments as a pharmaceutical (e.g., for the treatment of disorders which are mediated by TNF-alpha activity). For pharmaceutical use, one or more compounds of the preferred embodiments can be used, e.g., one, or a combination of two or more compounds of the preferred embodiments; however, preferably one compound is used. The compounds of preferred embodiments can be used as a pharmaceutical in the form of a pharmaceutical composition.

The TNF-alpha inhibitors can be employed therapeutically in compositions formulated for administration by any conventional route, including enterally (e.g., buccal, oral, nasal, rectal), parenterally (e.g., intravenous, intracranial, intraperitoneal, subcutaneous, or intramuscular), or topically (e.g., epicutaneous, intranasal, or intratracheal). Within other embodiments, the compositions described herein may be administered as part of a sustained release implant.

Within yet other embodiments, compositions of preferred embodiments may be formulized as a lyophilizate, utilizing appropriate excipients that provide stability as a lyophilizate, and subsequent to rehydration.

Pharmaceutical compositions containing the TNF-alpha inhibitors of preferred embodiments can be manufactured according to conventional methods, e.g., by mixing, granulating, coating, dissolving or lyophilizing processes.

In another embodiment, pharmaceutical compositions containing one or more TNF-alpha inhibitors are provided. For the purposes of administration, the compounds of preferred embodiments may be formulated as pharmaceutical compositions. Pharmaceutical compositions of preferred embodiments comprise one or more TNF-alpha inhibitors of preferred embodiments and a pharmaceutically acceptable carrier and/or diluent.

The TNF-alpha inhibitor is preferably employed in pharmaceutical compositions in an amount which is effective to treat a particular disorder, that is, in an amount sufficient to achieve decreased TNF-alpha levels or activity, symptoms, and/or preferably with acceptable toxicity to the patient. For such treatment, the appropriate dosage will, of course, vary depending upon, for example, the chemical nature and the pharmacokinetic data of a compound of the present invention used, the individual host, the mode of administration and the nature and severity of the conditions being treated. However, in general, for satisfactory results in larger mammals, for example humans, an indicated daily dosage is preferably from about 0.001 g to about 1.5 g, more preferably from about 0.01 g to 1.0 g; or from about 0.01 mg/kg body weight to about 20 mg/kg body weight, more preferably from about 0.1 mg/kg body weight to about 10 mg/kg body weight, for example, administered in divided doses up to four times a day. The compounds of preferred embodiments can be administered to larger mammals, for example humans, by similar modes of administration at similar dosages than conventionally used with other mediators, e.g., low molecular weight inhibitors, of TNF-alpha activity.

In certain embodiments, the pharmaceutical compositions of preferred embodiments can include TNF-alpha inhibitor(s) in an amount of about 0.5 mg or less to about 1500 mg or more per unit dosage form depending upon the route of administration, preferably from about 0.5, 0.6, 0.7, 0.8, or 0.9 mg to about 150, 200, 250, 300, 350, 400, 450, 500, 600, 700, 800, 900, or 1000 mg, and more preferably from about 1, 2, 3, 4, 5, 6, 7, 8, 9, 10, 15, 20, or 25 mg to about 30, 35, 40, 45, 50, 55, 60, 65, 70, 75, 80, 85, 90, 95, or 100 mg. In certain embodiments, however, lower or higher dosages than those mentioned above may be preferred. Appropriate concentrations and dosages can be readily determined by one skilled in the art.

Pharmaceutically acceptable carriers and/or diluents are familiar to those skilled in the art. For compositions formulated as liquid solutions, acceptable carriers and/or diluents include saline and sterile water, and may optionally include antioxidants, buffers, bacteriostats, and other common additives. The compositions can also be formulated as pills, capsules, granules, tablets (coated or uncoated), (injectable) solutions, solid solutions, suspensions, dispersions, solid dispersions (e.g., in the form of ampoules, vials, creams, gels, pastes, inhaler powder, foams, tinctures, lipsticks, drops, sprays, or suppositories). The formulation can contain (in addition to one or more TNF-alpha inhibitors and other optional active ingredients) carriers, fillers, disintegrators, flow conditioners, sugars and sweeteners, fragrances, preservatives, stabilizers, wetting agents, emulsifiers, solubilizers, salts for regulating osmotic pressure, buffers, diluents, dispersing and surface-active agents, binders, lubricants, and/or other pharmaceutical excipients as are known in the art. One skilled in this art may further formulate the TNF-alpha in an appropriate manner, and in accordance with accepted practices, such as those described in Remington's Pharmaceutical Sciences, Gennaro, Ed., Mack Publishing Co., Easton, Pa. 1990.

The compounds of preferred embodiments, including compounds of formula I, formula Iₚ, or other formulae above can include isomers, racemates, optical isomers, enantiomers, diastereomers, tautomers, and cis/trans conformers. All such isomeric forms are included within preferred embodiments, including mixtures thereof. The compounds of preferred embodiments may have chiral centers, for example, they may contain asymmetric carbon atoms and may thus exist in the form of enantiomers or diastereoisomers and mixtures thereof, e.g., racemates. Asymmetric carbon atom(s) can be present in the (R)-, (S)-, or (R,S)-configuration, preferably in the (R)- or (S)-configuration, or can be present as mixtures. Isomeric mixtures can be separated, as desired, according to conventional methods to obtain pure isomers. The compounds of preferred embodiments, e.g., formula I, can also include tautomers, where such tautomers can exist.

Furthermore, some of the crystalline forms of the compounds of preferred embodiments can exist as polymorphs, which are included in preferred embodiments. In addition, some of the compounds of preferred embodiments may also form solvates with water or other organic solvents. Such solvates are similarly included within the scope of the preferred embodiments.

In another embodiment, a method is provided for treating a variety of disorders or illnesses as described herein. Such methods include administering of a compound of preferred embodiments to a patient in an amount sufficient to treat the disorder or illness. Such methods include systemic administration of a TNF-alpha inhibitor of preferred embodiments, preferably in the form of a pharmaceutical composition. As used herein, systemic administration includes oral and parenteral methods of administration. For oral administration, suitable pharmaceutical compositions of an TNF-alpha inhibitor include powders, granules, pills, tablets, and capsules as well as liquids, syrups, suspensions, and emulsions. These compositions may also include flavorants, preservatives, suspending, thickening, and emulsifying agents, and other pharmaceutically acceptable additives. For parental administration, the compounds of preferred embodiments can be prepared in aqueous injection solutions that may contain, in addition to the TNF-alpha inhibitor, buffers, antioxidants, bacteriostats, and other additives commonly employed in such solutions.

As mentioned above, administration of a compound of preferred embodiments can be employed to treat a wide variety of disorders or illnesses. In particular, the compounds of preferred embodiments may be administered to a patient for the treatment of diseases and disorders as described above.

TNF-alpha inhibitors can be used alone, or in combination therapies with one, two, or more other pharmaceutical compounds or drug substances, and/or with one or more pharmaceutically acceptable excipient. The compounds of preferred embodiments and the additional pharmaceutical compounds or drug substances can be present in the same unit dosage form, or in two or more separate dosage forms. A method for treating disorders mediated by of TNF-alpha activity in a subject in need thereof is provided, comprising co-administering, concomitantly or in sequence, a therapeutically effective amount of a compound of the present invention and at least one second drug substance, e.g., in the form of a pharmaceutical combination or composition. Also provided is a compound of the preferred embodiments in combination with at least one second drug substance, e.g., in the form of a pharmaceutical combination or composition, for use in the preparation of a medicament for use in disorders mediated by TNF-alpha activity.

In preferred embodiments, the TNF-alpha inhibitor is present in combination with conventional drugs used to treat diseases or conditions wherein TNF-alpha is pathogenic or wherein TNF-alpha plays a pivotal or other role in the disease process. In particularly preferred embodiments, pharmaceutical compositions are provided comprising one or more TNF-alpha inhibitors, including, but not limited to compounds of the preferred embodiments in combination with one or more additional pharmaceutical compounds, including, but not limited to drugs for the treatment of various cancers, asthma or other respiratory diseases, diabetes sepsis, arthritis or other inflammatory diseases, immune disorders, or other diseases or disorders wherein TNF-alpha is pathogenic.

The TNF-alpha inhibitors of preferred embodiments can be used for pharmaceutical treatment alone or in combination with one or more other pharmaceutically active agents, e.g., such as agents useful in treating inflammation, tumor growth, or associated diseases. Such other pharmaceutically active agents include, e.g., steroids, glucocorticoids, inhibitors of other inflammatory cytokines (e.g., anti-TNF-alpha antibodies, anti-IL-1 antibodies, anti-IFN-γ antibodies), and other cytokines such as IL-IRA or IL-10, and other TNF-alpha inhibitors.

Combination therapies can include fixed combinations, in which two or more pharmaceutically active agents are in the same formulation; kits, in which two or more pharmaceutically active agents in separate formulations are sold in the same package, e.g., with instructions for co-administration; and free combinations in which the pharmaceutically active agents are packaged separately, but instruction for simultaneous or sequential administration are provided. Other kit components can include diagnostics, assays, multiple dosage forms for sequential or simultaneous administration, instructions and materials for reconstituting a lyophilized or concentrated form of the pharmaceutical composition, apparatus for administering the pharmaceutically active agents, and the like. For example, a pharmaceutical package is provided comprising a first drug substance which is a compound of the preferred embodiments and at least one second drug substance, along with instructions for combined administration. A pharmaceutical package is also provided comprising a compound of the preferred embodiments along with instructions for combined administration with at least one second drug substance. Also provided is a pharmaceutical package comprising at least one second drug substance along with instructions for combined administration with a compound of the present invention.

Treatment with combinations according to the preferred embodiments may provide improvements or superior outcome compared with treatments by either component of the combination alone. For example, a pharmaceutical combination comprising an amount of a compound of the preferred embodiments and an amount of a second drug substance can be employed, wherein the amounts are appropriate to produce a synergistic therapeutic effect. A method for improving the therapeutic utility of a compound of the preferred embodiments is also provided, comprising co-administering, e.g., concomitantly or in sequence, a therapeutically effective amount of a compound of the preferred embodiments and a second drug substance. A method for improving the therapeutic utility of a second drug substance is also provided comprising co-administering, e.g., concomitantly or in sequence, a therapeutically effective amount of a compound of the preferred embodiments and a second drug substance. A combination of the present invention and a second drug substance as a combination partner can be administered by any conventional route, for example as set out above for a compound of the preferred embodiments. A second drug can be administered in dosages as appropriate, e.g., in dosage ranges which are similar to those used for single treatment, or, e.g., in case of synergy, even below conventional dosage ranges.

Suitable second drug substances include chemotherapeutic drugs, especially any chemotherapeutic agent other than the TNF-alpha inhibitors of preferred embodiments. Such second drug substances can include, e.g., anti-inflammatory and/or immunomodulatory drugs, anticancer drugs, and the like.

Anti-inflammatory and/or immunomodulatory drugs which may be used in combination with a compound of formula I include e.g., mTOR inhibitors, including rapamycins, e.g., rapamycin of formula:

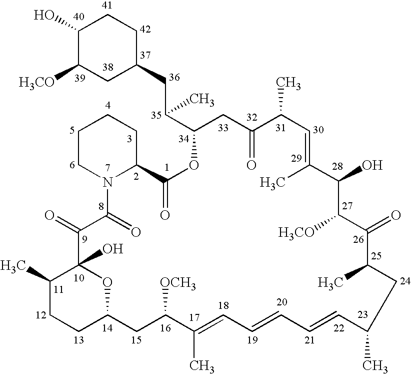

40-O-(2-hydroxyethyl)-rapamycin, 32-deoxorapamycin, 16-O-substituted rapamycins such as 16-pent-2-ynyloxy-32-deoxorapamycin, 16-pent-2-ynyloxy-32 (S or R)-dihydro-rapamycin, 16-pent-2-ynyloxy-32(S or R)-dihydro-40-O-(2-hydroxyethyl)-rapamycin, 40-[3-hydroxy-2-(hydroxy-methyl)-2-methylpropanoate]-rapamycin (also known as CC1779), 40-epi-(tetrazolyl)-rapamycin (also known as ABT578), the so-called rapalogs, e.g., as disclosed in PCT International Application No. WO98/02441, PCT International Application No. WO01/14387, and PCT International Application No. WO03/64383, such as AP23573, and compounds disclosed under the name TAFA-93 and biolimus (biolimus A9); calcineurin inhibitors, e.g., cyclosporin A or FK 506; ascomycins having immuno-suppressive properties, e.g., ABT-281, ASM981; corticosteroids; cyclophosphamide; azathioprene; leflunomide; mizoribine; mycophenolic acid or salt; mycophenolate mofetil; 15-deoxyspergualine or an immunosuppressive homologue, analogue or derivative thereof, bcr-abl tyrosine kinase inhibitors; c-kit receptor tyrosine kinase inhibitors; PDGF receptor tyrosine kinase inhibitors, e.g., Gleevec (imatinib); p38 MAP kinase inhibitors, VEGF receptor tyrosine kinase inhibitors, PKC inhibitors, e.g., as disclosed in PCT International Application No. WO02/38561 or PCT International Application No. WO03/82859, e.g., the compound of Example 56 or 70; JAK3 kinase inhibitors, e.g., N-benzyl-3,4-dihydroxy-benzylidene-cyanoacetamide α-cyano-(3,4-dihydroxy)-]N-benzylcinnamamide (Tyrphostin AG 490), prodigiosin 25-C(PNU156804), [4-(4′-hydroxyphenyl)-amino-6,7-dimethoxyquinazoline] (WHI-P131), [4-(3′-bromo-4′-hydroxylphenyl)-amino-6,7-dimethoxyquinazoline] (WHI-P154), [4-(3′,5′-dibromo-4′-hydroxylphenyl)-amino-6,7-dimethoxyquinazoline] WHI-P197, KRX-211, 3-{(3R,4R)-4-methyl-3-[methyl-(7H-pyrrolo[2,3-d]pyrimidin-4-yl)-amino]-piperidin-1-yl}-3-oxo-propionitrile, in free form or in a pharmaceutically acceptable salt form, e.g., mono-citrate (also called CP-690,550), or a compound as disclosed in PCT International Application No. WO2004/052359 or PCT International Application No. WO2005/066156; SIP receptor agonists or modulators, e.g., FTY720 optionally phosphorylated or an analog thereof, e.g., 2-amino-2-[4-(3-benzyloxyphenylthio)-2-chlorophenyl]ethyl-1,3-propanediol optionally phosphorylated or 1-{4-[1-(4-cyclohexyl-3-trifluoromethyl-benzyloxyimino)-ethyl]-2-ethyl-benzyl}-azetidine-3-carboxylic acid or its pharmaceutically acceptable salts; immunosuppressive monoclonal antibodies, e.g., monoclonal antibodies to leukocyte receptors, e.g., Blys/BAFF receptor, MHC, CD2, CD3, CD4, CD7, CD8, CD25, CD28, CD40, CD45, CD52, CD58, CD80, CD86, IL-12 receptor, IL-17 receptor, IL-23 receptor or their ligands; other immunomodulatory compounds, e.g., a recombinant binding molecule having at least a portion of the extracellular domain of CTLA4 or a mutant thereof, e.g., an at least extracellular portion of CTLA4 or a mutant thereof joined to a non-CTLA4 protein sequence, e.g., CTLA4Ig (for ex. designated ATCC 68629) or a mutant thereof, e.g., LEA29Y; adhesion molecule inhibitors, e.g., LFA-1 antagonists, ICAM-1 or -3 antagonists, VCAM-4 antagonists or VLA-4 antagonists, CCR9 antagonists, MIF inhibitors, 5-aminosalicylate (5-ASA) agents, such as sulfasalazine, Azulfidine®, Asacol®, Dipentum®, Pentasa®, Rowasa®, Canasa®, Colazal®, e.g., drugs containing mesalamine; e.g., mesalazine in combination with heparin; TNF-alpha inhibitors, e.g., others than those of the present invention, such as antibodies which bind to TNF-alpha, e.g., infliximab (Remicade®), nitric oxide releasing non-steroidal anti-inflammatory drugs (NSAIDs), e.g., including COX-inhibiting NO-donating drugs (CINOD); phosphodiesterase, e.g., PDE4B-inhibitors, caspase inhibitors, ‘multi-functional anti-inflammatory’ drugs (MFAIDs), e.g., cytosolic phospholipase A2 (cPLA2) inhibitors, such as membrane-anchored phospholipase A2 inhibitors linked to glycosaminoglycans.

Anticancer drug useful as combination partners with a compound of formula I, e.g., include drugs such as disclosed as “chemotherapeutic agents” in PCT International Application No. WO02/066019, e.g., on pages 5 and 6 under i) to x), in more detail on pages 6 to 11, namely agents which are disclosed to be useful in combination treatment of solid tumors, permetrexed (Alimta®), sunitinib (SU11248), temozolidine, daunorubicil, dactinomycm, doxorubicin, bleomycin, mitomycin, nitrogen mustard, chlorambucil, melphalan, cyclophosphamide, 6-mercaptopurine, 6-thioguanine, cytarabine (CA), 5-fluorouracil(5-FU), floxuridine (5-FUdR), methotrexate (MTX), colchicine, vincristine, vinblastine, etoposide, teniposide, cisplatin, diethylstilbestrol (DES).

In other preferred embodiments, one or more TNF-alpha inhibiting compounds of the preferred embodiments are present in combination with one or more nonsteroidal anti-inflammatory drugs (NSAIDs) or other pharmaceutical compounds for treating arthritis or other inflammatory diseases. Preferred compounds include, but are not limited to, celecoxib; rofecoxib; NSAIDS, for example, aspirin, celecoxib, choline magnesium trisalicylate, diclofenac potassium, diclofenac sodium, diflunisal, etodolac, fenoprofen, flurbiprofen, ibuprofen, indomethacin, ketoprofen, ketorolac, melenamic acid, nabumetone, naproxen, naproxen sodium, oxaprozin, piroxicam, rofecoxib, salsalate, sulindac, and tolmetin; and corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone.

In particularly preferred embodiments, one or more TNF-alpha inhibiting compounds are present in combination with one or more beta stimulants, inhalation corticosteroids, antihistamines, hormones, or other pharmaceutical compounds for treating asthma, acute respiratory distress, or other respiratory diseases. Preferred compounds include, but are not limited to, beta stimulants, for example, commonly prescribed bronchodilators; inhalation corticosteroids, for example, beclomethasone, fluticasone, triamcinolone, mometasone, and forms of prednisone such as prednisone, prednisolone, and methylprednisolone; antihistamines, for example, azatadine, carbinoxamine/pseudoephedrine, cetirizine, cyproheptadine, dexchlorpheniramine, fexofenadine, loratadine, promethazine, tripelennamine, brompheniramine, cholopheniramine, clemastine, diphenhydramine; and hormones, for example, epinephrine.

In particularly preferred embodiments, one or more TNF-alpha inhibiting compounds are present in combination with one or more anesthetics, e.g., ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine.

In particularly preferred embodiments, one or more TNF-alpha inhibiting compounds are present in combination with pharmaceutical compounds for treating irritable bowel disease, such as azathioprine or corticosteroids, in a pharmaceutical composition.

In particularly preferred embodiments, one or more TNF-alpha inhibiting compounds are present in combination with pharmaceutical compounds for treating cancer, such as paclitaxel, in a pharmaceutical composition.

In particularly preferred embodiments, one or more TNF-alpha inhibiting compounds are present in combination with immunosuppresive compounds in a pharmaceutical composition. In particularly preferred embodiments, one or more TNF-alpha inhibiting compounds are present in combination with one or more drugs for treating an autoimmune disorder, for example, Acquired Immune Deficiency Syndrome (AIDS). Such drugs may include protease inhibitors, for example, indinavir, amprenavir, saquinavir, lopinavir, ritonavir, and nelfinavir; nucleoside reverse transcriptase inhibitors, for example, zidovudine, abacavir, lamivudine, idanosine, zalcitabine, and stavudine; nucleotide reverse transcriptase inhibitors, for example, tenofovir disoproxil fumarate; non nucleoside reverse transcriptase inhibitors, for example, delavirdine, efavirenz, and nevirapine; biological response modifiers, for example, etanercept, infliximab, and other compounds that inhibit or interfere with tumor necrosing factor; antivirals, for example, amivudine and zidovudine.

In particularly preferred embodiments, one or more TNF-alpha inhibiting compounds are present in combination with pharmaceutical compounds for treating sepsis, such as steroids or anti-infective agents. Examples of steroids include corticosteroids, for example, cortisone, hydrocortisone, methylprednisolone, prednisone, prednisolone, betamethesone, beclomethasone dipropionate, budesonide, dexamethasone sodium phosphate, flunisolide, fluticasone propionate, triamcinolone acetonide, betamethasone, fluocinolone, fluocinonide, betamethasone dipropionate, betamethasone valerate, desonide, desoximetasone, fluocinolone, triamcinolone, triamcinolone acetonide, clobetasol propionate, and dexamethasone. Examples of anti-infective agents include anthelmintics (mebendazole), antibiotics including aminoglycosides (gentamicin, neomycin, tobramycin), antifungal antibiotics (amphotericin b, fluconazole, griseofulvin, itraconazole, ketoconazole, nystatin, micatin, tolnaftate), cephalosporins (cefaclor, cefazolin, cefotaxime, ceftazidime, ceftriaxone, cefuroxime, cephalexin), beta-lactam antibiotics (cefotetan, meropenem), chloramphenicol, macrolides (azithromycin, clarithromycin, erythromycin), penicillins (penicillin G sodium salt, amoxicillin, ampicillin, dicloxacillin, nafcillin, piperacillin, ticarcillin), tetracyclines (doxycycline, minocycline, tetracycline), bacitracin; clindamycin; colistimethate sodium; polymyxin b sulfate; vancomycin; antivirals including acyclovir, amantadine, didanosine, efavirenz, foscarnet, ganciclovir, indinavir, lamivudine, nelfinavir, ritonavir, saquinavir, stavudine, valacyclovir, valganciclovir, zidovudine; quinolones (ciprofloxacin, levofloxacin); sulfonamides (sulfadiazine, sulfisoxazole); sulfones (dapsone); furazolidone; metronidazole; pentamidine; sulfanilamidum crystallinum; gatifloxacin; and sulfamethoxazole/trimethoprim, pleuromutilins, fluoroquinolones, e.g., metronidazole, quinolones such as ciprofloxacin; levofloxacin; probiotics and commensal bacteria e.g., Lactobacillus, Lactobacillus reuteri; antiviral drugs, such as ribivirin, vidarabine, acyclovir, ganciclovir, zanamivir, oseltamivir phosphate, famciclovir, atazanavir, amantadine, didanosine, efavirenz, foscarnet, indinavir, lamivudine, nelfinavir, ritonavir, saquinavir, stavudine, valacyclovir, valganciclovir, zidovudine.

In the treatment of certain diseases, it may be beneficial to treat the patient with a TNF-alpha inhibitor in combination with an anesthetic, for example, ethanol, bupivacaine, chloroprocaine, levobupivacaine, lidocaine, mepivacaine, procaine, ropivacaine, tetracaine, desflurane, isoflurane, ketamine, propofol, sevoflurane, codeine, fentanyl, hydromorphone, marcaine, meperidine, methadone, morphine, oxycodone, remifentanil, sufentanil, butorphanol, nalbuphine, tramadol, benzocaine, dibucaine, ethyl chloride, xylocaine, and phenazopyridine.

These compounds of preferred embodiments can generally be employed as the free acid or the free base. Alternatively, the compounds of preferred embodiments can preferably be in the form of acid or base addition salts. The term “pharmaceutically acceptable salt” of compounds of the preferred embodiments is intended to encompass any and all acceptable salt forms. While salt forms of the preferred embodiments are preferably pharmaceutically acceptable salts, in certain embodiments pharmaceutically unacceptable salts can be employed (e.g., for preparation, isolation, and/or purification purposes). The compounds of preferred embodiments can also be employed in the form of a solvate, or in various combinations of forms (free acid, free base, salt, and/or solvate). A compound of the preferred embodiments in free form can be converted into a corresponding compound in the form of a salt; and vice versa. A solvate of a compound of preferred embodiments in free form or in the form of a salt can be converted into a corresponding non-solvate form of the compound in free form or in the form of a salt; and vice versa.

The compounds of preferred embodiments can be made according to the organic synthesis techniques known to those skilled in this field, as well as by the representative methods set forth in the following examples, or according to methods, with appropriate modification(s) if necessary, as set forth in PCT International Application No. WO02/094203 (see, e.g., Scheme 4 on page 34), PCT International Application No. WO2004/074218 or PCT International Application No. WO2005/021546 (see, e.g., Scheme 4 on page 113, Scheme 6 on page 115).

### Preparation of Compounds of Structure (I)

To prepare compounds of structure (I):

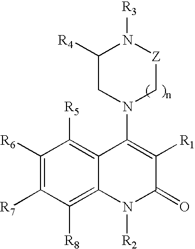

appropriately substituted or unsubstituted isatoic anhydride can be used as starting material. The isatoic anhydride can react with appropriately substituted or unsubstituted compounds having active methylene group, depicted by general formula (1) to yield substituted or unsubstituted 4-hydroxy-2-oxo-1,2-dihydro-quinoline intermediate, depicted by general formula (2) below. This intermediate can be reacted with phosphorus oxychloride to give substituted or unsubstituted 4-chloro-2-oxo-1,2-dihydro quinoline intermediate, depicted by general formula (3) as shown in Scheme 1.

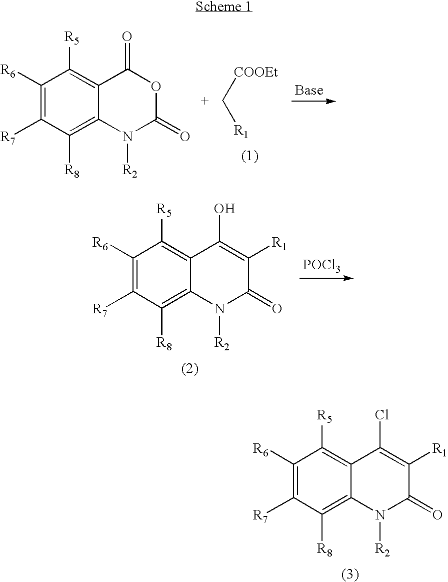

In one method substituted or unsubstituted 4-chloro-2-oxo-1,2-dihydro-quinoline intermediate, depicted by general formula (3) can be reacted with piperazine at room temperature to get compounds of structure (I) with R₃ as hydrogen as shown in Scheme 2. In another method, substituted or unsubstituted 4-chloro-2-oxo-1,2-dihydro-quinoline intermediate, depicted by general formula (3) can be reacted with substituted or unsubstituted piperazine derivative to get compounds of structure (I). In some cases, the substitution R₃ can be introduced directly to substituted or unsubstituted 4-(piperazin-1-yl)-quinolinone intermediate, depicted by formula (4) by reacting with appropriate halides to get compounds of structure (I) with R₃ as defined above.

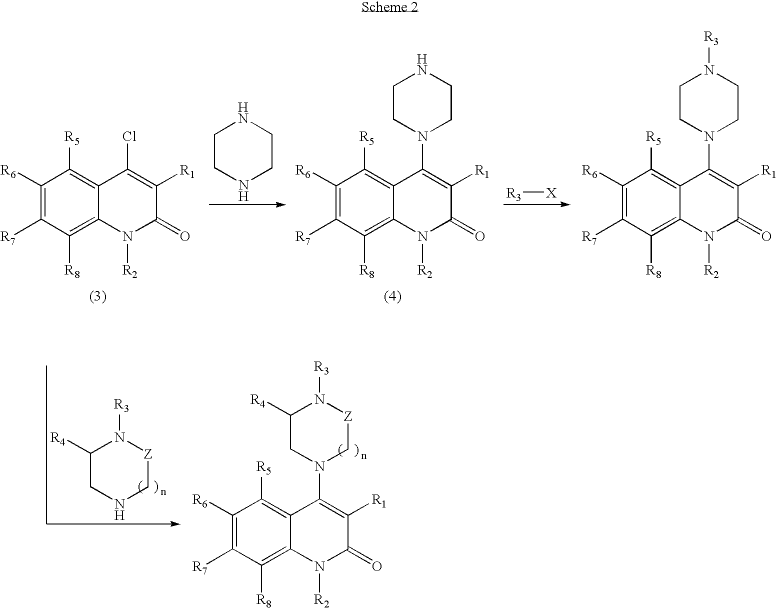

In an alternative method to prepare compound of general formula (I) with R₁ as nitrile substituted or unsubstituted 4-chloro-2-oxo-1,2-dihydro-quinoline-3-carbonitrile intermediate, depicted by formula (7) can be used as a preferred intermediate. To prepare this intermediate appropriately substituted or unsubstituted isatoic anhydride can be reacted with ethyl malonate to get substituted or unsubstituted 4-hydroxy-2-oxo-1,2-dihydro-quinoline intermediate, depicted by formula (5) as shown in Scheme 3. The 4-hydroxy-2-oxo-1,2-dihydro-quinoline intermediate, depicted by formula (5) can be converted into corresponding amide intermediate, depicted by formula (6), which can further react with phosphorus oxychloride to yield substituted or unsubstituted 4-chloro-2-oxo-1,2-dihydro-quinoline-3-carbonitrile intermediate, depicted by formula (7) as shown in Scheme 3. This intermediate can be then reacted with piperazine or substituted or unsubstituted piperazine derivatives to get compounds of general structure (I) with R₁ as nitrile as shown in Scheme 3.

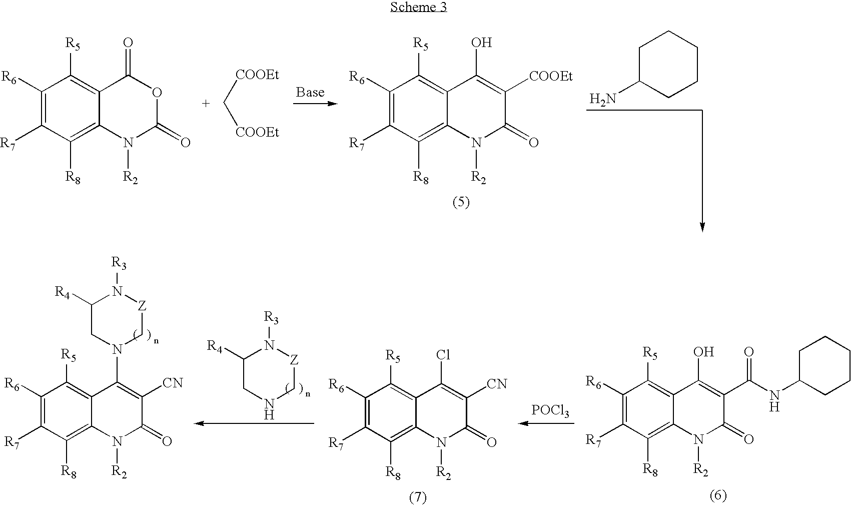

### Preparation of Compounds of Structure (II)

To prepare compounds of structure (II):

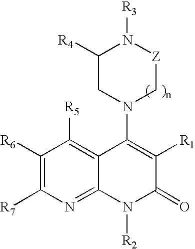

appropriately substituted or unsubstituted 2-chloro nicotinic acid can be reacted with appropriate amine to yield substituted or unsubstituted 2-amino nicotinic acid intermediate, depicted by formula (8). Substituted or unsubstituted 2-amino nicotinic acid intermediate, depicted by formula (8) can be reacted with trichloromethyl chloroformate to yield intermediate of general structure (9) as shown in Scheme 4. This intermediate can then react with compound having active methylene group to yield corresponding substituted or unsubstituted 4-hydroxy-2-oxo-1,2-dihydro-[1,8]-naphthyridine intermediate, depicted by general formula (10) as shown in Scheme 4. Substituted or unsubstituted 4-hydroxy-2-oxo-1,2-dihydro-[1,8]-naphthyridine intermediate, depicted by general formula (10), can yield 4-chloro-2-oxo-1,2-dihydro-[1,8]-naphthyridine intermediate, depicted by formula (11), by reacting with phosphorus oxychloride as shown in Scheme 4.

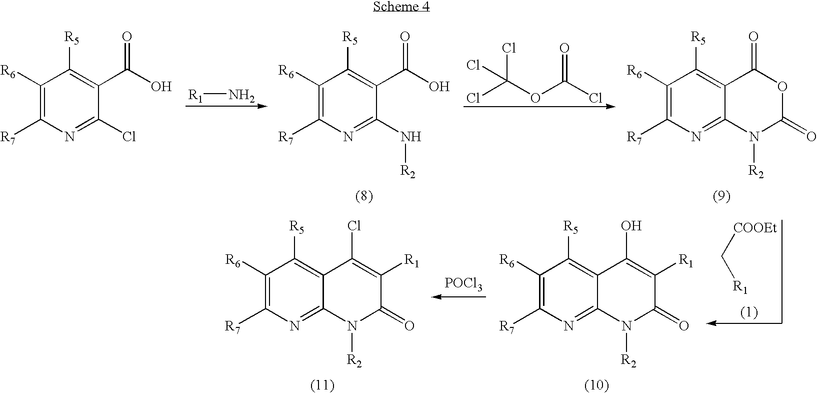

In one method substituted or unsubstituted 4-chloro-2-oxo-1,2-dihydro-[1,8]-naphthyridine intermediate, depicted by general formula (11) can be reacted with piperazine at room temperature to yield compounds of structure (II) with R₃ as hydrogen as shown in Scheme 5. In another method, substituted or unsubstituted 4-chloro-2-oxo-1,2-dihydro-[1,8]-naphthyridine intermediate, depicted by general formula (11) can be reacted with substituted or unsubstituted piperazine derivative to yield compounds of structure (II). In some cases, the substitution R₃ was introduced directly to substituted or unsubstituted 2-oxo-1,2-dihydro-4-(piperazin-1-yl)-[1,8]-naphthyridine intermediate, depicted by formula (12) by reacting with appropriate halides to yield compounds of structure (II) with R₃ as defined above.

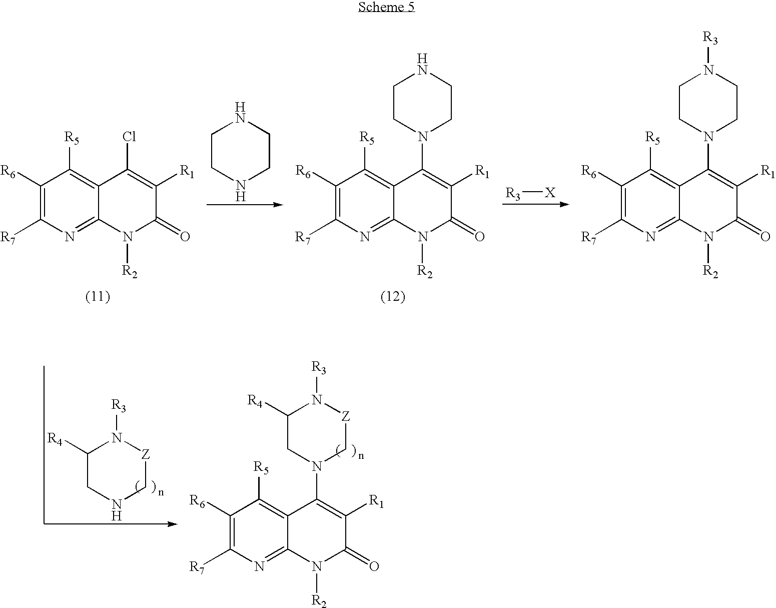

In an alternative method to prepare compound of general formula (II) with R₁ as nitrile substituted or unsubstituted 4-chloro-2-oxo-1,2-dihydro-[1,8]-naphthyridine-3-carbonitrile intermediate, depicted by formula (15) can be used as a preferred intermediate. To prepare this intermediate appropriately substituted or unsubstituted 1H-pyrido[3,4-d]oxazine-2,4-dione, depicted by formula (9) can be reacted with ethyl malonate to yield substituted or unsubstituted 4-hydroxy-2-oxo-1,2-dihydro-[1,8]-naphthyridine intermediate, depicted by formula (13) as shown in Scheme 6. The substituted or unsubstituted 4-hydroxy-2-oxo-1,2-dihydro-[1,8]-naphthyridine intermediate, depicted by formula (13) can be converted into corresponding amide intermediate, depicted by formula (14), which can further react with phosphorus oxychloride to yield substituted or unsubstituted 4-chloro-2-oxo-1,2-dihydro-[1,8]-naphthyridine-3-carbonitrile intermediate, depicted by formula (15) as shown in Scheme 6. This intermediate was then reacted with piperazine or substituted or unsubstituted piperazine derivatives to get compounds of general structure (II) with R₁ as nitrile as shown in Scheme 6.

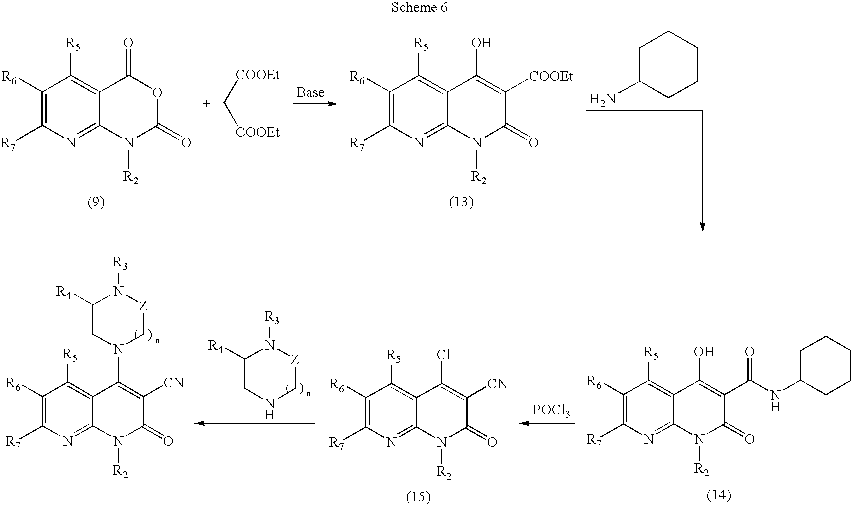

### Preparation of Compounds of General Structure (III)

Preferred compounds of general structure (III):

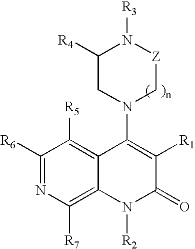

can be prepared by using substituted or unsubstituted pyridine 3,4-dicarboxylic acid as a starting material. Substituted or unsubstituted pyridine 3,4-dicarboxylic acid can react with acetic anhydride to give substituted or unsubstituted furo[3,4-c]pyridine-1,3-dione, depicted by formula (16) in Scheme 7, which can be converted to substituted or unsubstituted pyrrolo[3,4-c]pyridine-1,3-dione, depicted by formula (17) in Scheme 7, by reacting with acetamide. Substituted or unsubstituted 3-amino isonicotinic acid can be prepared from Hoffmann degradation of this intermediate. Reductive amination of substituted or unsubstituted 3-amino isonicotinic acid can give substituted or unsubstituted 3-alkylamino isonicotinic acid, depicted by formula (19) in Scheme 7. This intermediate can also be prepared from alkylation of 3-amino isonicotinic acid by using lithium hexamethyl disilazide and corresponding halides as shown in Scheme 7. Substituted or unsubstituted 3-alkylamino isonicotinic acid, depicted by formula (19) can react with trichloromethyl chloroformate to yield substituted or unsubstituted 1H-pyrido[3,4-d][1,3]oxazine-2,4-dione depicted by formula (20) as shown in Scheme 7.

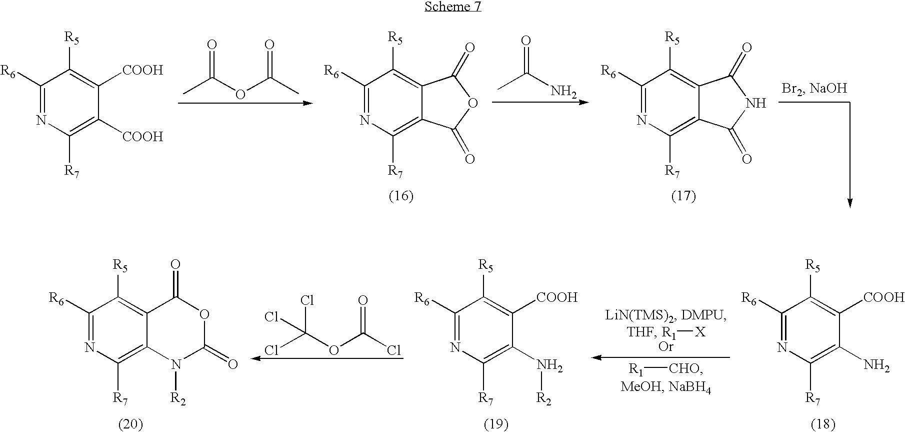

The substituted or unsubstituted 1H-pyrido[3,4-d][1,3]oxazine-2,4-dione depicted by formula (20) intermediate can then react with compound having active methylene group to yield corresponding substituted or unsubstituted 4-hydroxy-2-oxo-1,2-dihydro-[1,7]-naphthyridine intermediate, depicted by general formula (21) as shown in Scheme 8. Substituted or unsubstituted 4-hydroxy-2-oxo-1,2-dihydro-[1,7]-naphthyridine intermediate, depicted by general formula (21), can yield 4-chloro-2-oxo-1,2-dihydro-[1,7]-naphthyridine intermediate, depicted by formula (22), by reacting with phosphorus oxychloride as shown in Scheme 8.

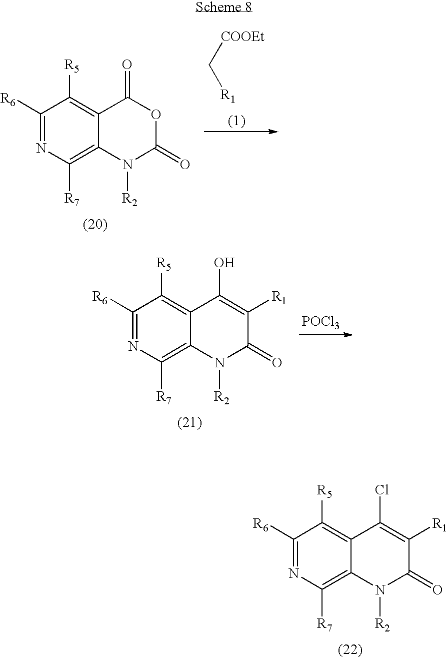

In one method substituted or unsubstituted 4-chloro-2-oxo-1,2-dihydro-[1,7]-naphthyridine intermediate, depicted by general formula (22) can be reacted with piperazine at room temperature to yield compounds of structure (III) with R₃ as hydrogen as shown in Scheme 9. In another method, substituted or unsubstituted 4-chloro-2-oxo-1,2-dihydro-[1,7]-naphthyridine intermediate, depicted by general formula (22) can be reacted with substituted or unsubstituted piperazine derivative to yield compounds of structure (III). In some cases, the substitution R₃ was introduced directly to substituted or unsubstituted 2-oxo-1,2-dihydro-4-(piperazin-1-yl)-[1,7]-naphthyridine intermediate, depicted by formula (23) by reacting with appropriate halides to yield compounds of structure (III) with R₃ as defined above.

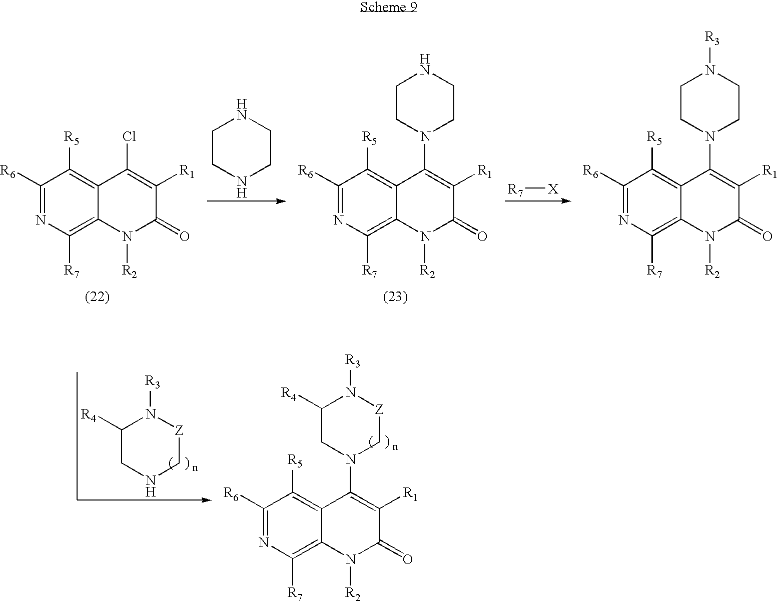

In an alternative method to prepare compound of general formula (III) with R₁ as nitrile substituted or unsubstituted 4-chloro-2-oxo-1,2-dihydro-[1,7]-naphthyridine-3-carbonitrile intermediate, depicted by formula (26) can be used as a preferred intermediate. To prepare this intermediate appropriately substituted or unsubstituted 1H-pyrido[3,4-d]oxazine-2,4-dione, depicted by formula (20) can be reacted with ethyl malonate to yield substituted or unsubstituted 4-hydroxy-2-oxo-1,2-dihydro-[1,7]-naphthyridine intermediate, depicted by formula (24) as shown in Scheme 10. The substituted or unsubstituted 4-hydroxy-2-oxo-1,2-dihydro-[1,7]-naphthyridine intermediate, depicted by formula (24) can be converted into corresponding amide intermediate, depicted by formula (25), which can further react with phosphorus oxychloride to yield substituted or unsubstituted 4-chloro-2-oxo-1,2-dihydro-[1,7]-naphthyridine-3-carbonitrile intermediate, depicted by formula (26) as shown in Scheme 10. This intermediate was then reacted with piperazine or substituted or unsubstituted piperazine derivatives to get compounds of general structure (III) with R₁ as nitrile as shown in Scheme 10.

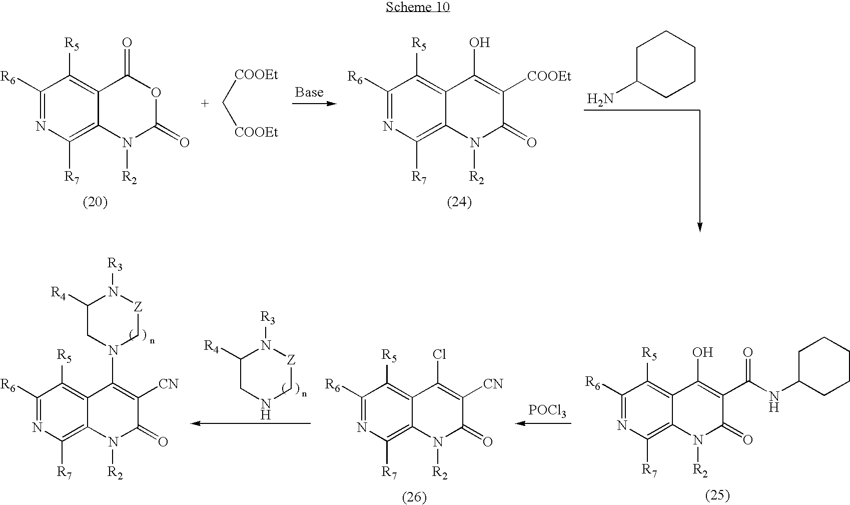

### Preparation of Compounds of General Structure (Iv)

To prepare compounds of structure (IV):

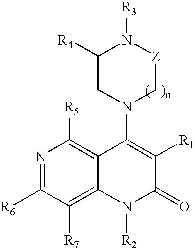

appropriately substituted or unsubstituted 4-aminopyridine can be used as starting material. Substituted or unsubstituted 4-aminopyridine can be protected by boc group and converted to substituted or unsubstituted 4-tert-butoxycarbonylamino-nicotinic acid, depicted as formula (27) in Scheme 11, by ortholithiation followed by quenching with dry ice. This intermediate can be reacted with trichloromethyl chloroformate to yield substituted or unsubstituted 1H-pyrido[4,3-d][1,3]oxazine-2,4-dione, depicted by formula (28) in Scheme 11, which can be then converted to substituted or unsubstituted 4-chloro-2-oxo-1,2-dihydro-[1,6]naphthyridine-3-carboxylic acid ethyl ester, depicted by formula (29) as shown in Scheme 11.

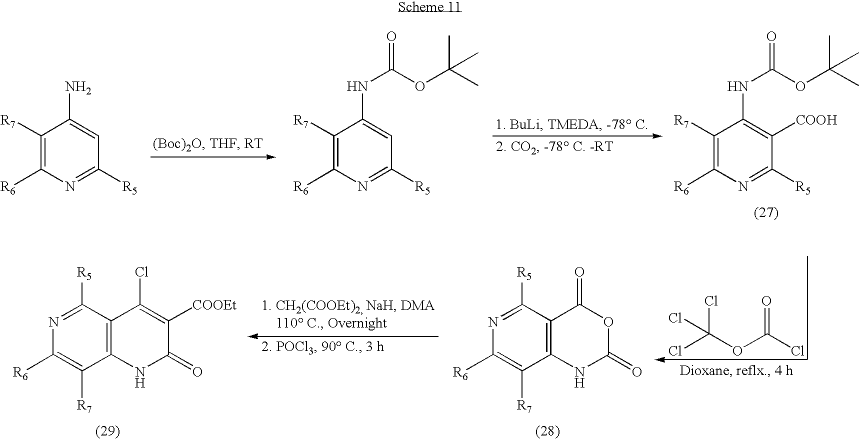

In one method substituted or unsubstituted 4-chloro-2-oxo-1,2-dihydro-[1,6]-naphthyridine intermediate, depicted by general formula (29) can be reacted with piperazine at room temperature to yield compounds of structure (IV) with R₃ as hydrogen as shown in Scheme 12. In another method, substituted or unsubstituted 4-chloro-2-oxo-1,2-dihydro-[1,6]-naphthyridine intermediate, depicted by general formula (29) can be reacted with substituted or unsubstituted piperazine derivative to yield compounds of structure (IV). In some cases, the substitution R₃ was introduced directly to substituted or unsubstituted 2-oxo-1,2-dihydro-4-(piperazin-1-yl)-[1,6]-naphthyridine intermediate, depicted by formula (30) by reacting with appropriate halides to yield compounds of structure (IV) with R₃ as defined above.

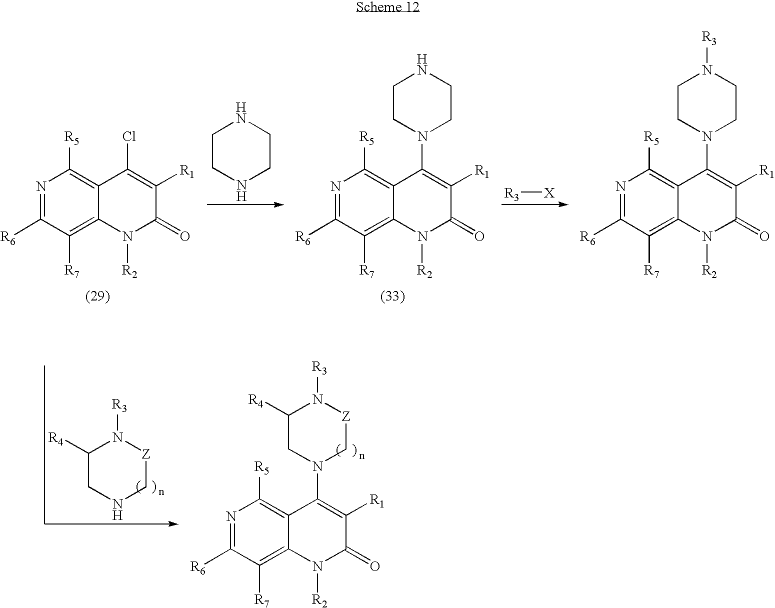

In an alternative method to prepare compound of general formula (IV) with R₁ as nitrile substituted or unsubstituted 4-chloro-2-oxo-1,2-dihydro-[1,6]-naphthyridine-3-carbonitrile intermediate, depicted by formula (33) can be used as a preferred intermediate. To prepare this intermediate appropriately substituted or unsubstituted 1H-pyrido[4,3-d][1,3]oxazine-2,4-dione, depicted by formula (28) in Scheme 13 can be reacted with ethyl malonate to yield substituted or unsubstituted 4-hydroxy-2-oxo-1,2-dihydro-[1,6]-naphthyridine intermediate, depicted by formula (31) as shown in Scheme 13. The substituted or unsubstituted 4-hydroxy-2-oxo-1,2-dihydro-[1,6]-naphthyridine intermediate, depicted by formula (31) can be converted into corresponding amide intermediate, depicted by formula (32), which can further react with phosphorus oxychloride to yield substituted or unsubstituted 4-chloro-2-oxo-1,2-dihydro-[1,6]-naphthyridine-3-carbonitrile intermediate, depicted by formula (33) as shown in Scheme 13. This intermediate was then reacted with piperazine or substituted or unsubstituted piperazine derivatives to get compounds of general structure (IV) with R₁ as nitrile as shown in Scheme 13.

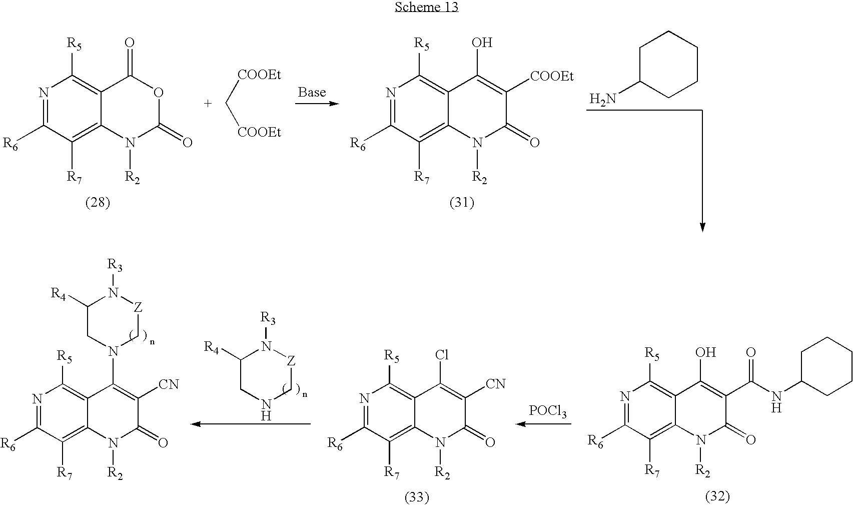

### Preparation of Compounds of General Structure (V)

Preferred compounds of general structure (V):

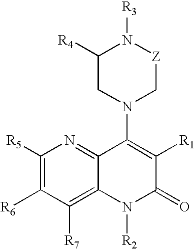

can be prepared by using substituted or unsubstituted pyridine 2,3-dicarboxylic acid as a starting material. Substituted or unsubstituted pyridine 2,3-dicarboxylic acid can react with acetic anhydride to give substituted or unsubstituted furo[3,4-b]pyridine-5,7-dione, depicted by formula (34) in Scheme 14, which can be converted to substituted or unsubstituted pyrrolo[3,4-b]pyridine-5,7-dione, depicted by formula (35) in Scheme 14, by reacting with acetamide. Substituted or unsubstituted 3-amino pyridine-2-carboxylic acid can be prepared from Hoffmann degradation of this intermediate. Reductive amination of substituted or unsubstituted 3-amino pyridine-2-carboxylic acid can give substituted or unsubstituted 3-alkylamino pyridine-2-carboxylic acid, depicted by formula (37) in Scheme 14. This intermediate can also be prepared from alkylation of 3-amino pyridine-2-carboxylic acid by using lithium hexamethyl disilazide and corresponding halides as shown in Scheme 14. Substituted or unsubstituted 3-alkylamino pyridine-2-carboxylic acid, depicted by formula (37) can react with trichloromethyl chloroformate to yield substituted or unsubstituted 1H-pyrido[3,2-d][1,3]oxazine-2,4-dione depicted by formula (38) as shown in Scheme 14.

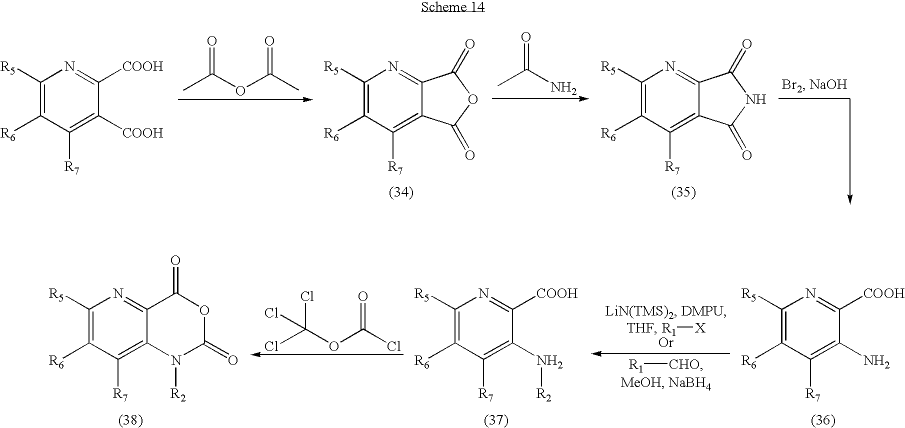

The substituted or unsubstituted 1H-pyrido[3,2-d][1,3]oxazine-2,4-dione depicted by formula (38) intermediate can then react with compound having active methylene group to yield corresponding substituted or unsubstituted 4-hydroxy-2-oxo-1,2-dihydro-[1,5]-naphthyridine intermediate, depicted by general formula (39) as shown in Scheme 15. Substituted or unsubstituted 4-hydroxy-2-oxo-1,2-dihydro-[1,5]-naphthyridine intermediate, depicted by general formula (39), can yield 4-chloro-2-oxo-1,2-dihydro-[1,5]-naphthyridine intermediate, depicted by formula (40), by reacting with phosphorus oxychloride as shown in Scheme 15.

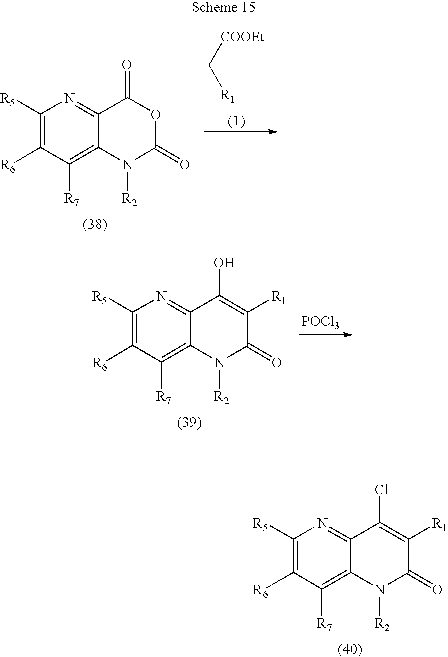

In one method substituted or unsubstituted 4-chloro-2-oxo-1,2-dihydro-[1,5]-naphthyridine intermediate, depicted by general formula (40) can be reacted with piperazine at room temperature to yield compounds of structure (V) with R₃ as hydrogen as shown in Scheme 16. In another method, substituted or unsubstituted 4-chloro-2-oxo-1,2-dihydro-[1,5]-naphthyridine intermediate, depicted by general formula (40) can be reacted with substituted or unsubstituted piperazine derivative to yield compounds of structure (V). In some cases, the substitution R₇ was introduced directly to substituted or unsubstituted 2-oxo-1,2-dihydro-4-(piperazin-1-yl)-[1,5]-naphthyridine intermediate, depicted by formula (41) by reacting with appropriate halides to yield compounds of structure (V) with R₃ as defined above.

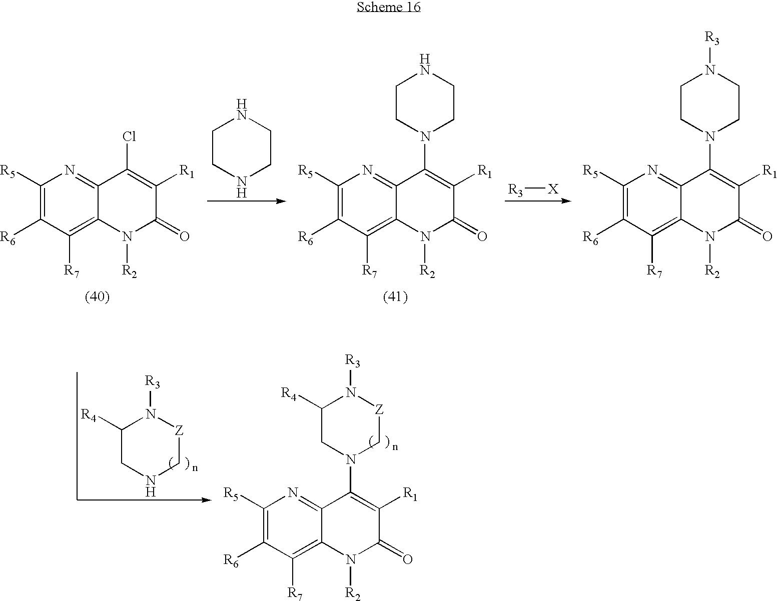

In an alternative method to prepare compound of general formula (V) with R₁ as nitrile substituted or unsubstituted 4-chloro-2-oxo-1,2-dihydro-[1,5]-naphthyridine-3-carbonitrile intermediate, depicted by formula (44) can be used as a preferred intermediate. To prepare this intermediate appropriately substituted or unsubstituted 1H-pyrido[3,2-d]oxazine-2,4-dione, depicted by formula (38) can be reacted with ethyl malonate to yield substituted or unsubstituted 4-hydroxy-2-oxo-1,2-dihydro-[1,5]-naphthyridine intermediate, depicted by formula (42) as shown in Scheme 17. The substituted or unsubstituted 4-hydroxy-2-oxo-1,2-dihydro-[1,5]-naphthyridine intermediate, depicted by formula (42) can be converted into corresponding amide intermediate, depicted by formula (43), which can further react with phosphorus oxychloride to yield substituted or unsubstituted 4-chloro-2-oxo-1,2-dihydro-[1,5]-naphthyridine-3-carbonitrile intermediate, depicted by formula (44) as shown in Scheme 17. This intermediate was then reacted with piperazine or substituted or unsubstituted piperazine derivatives to get compounds of general structure (V) with R₁ as nitrile as shown in Scheme 17.

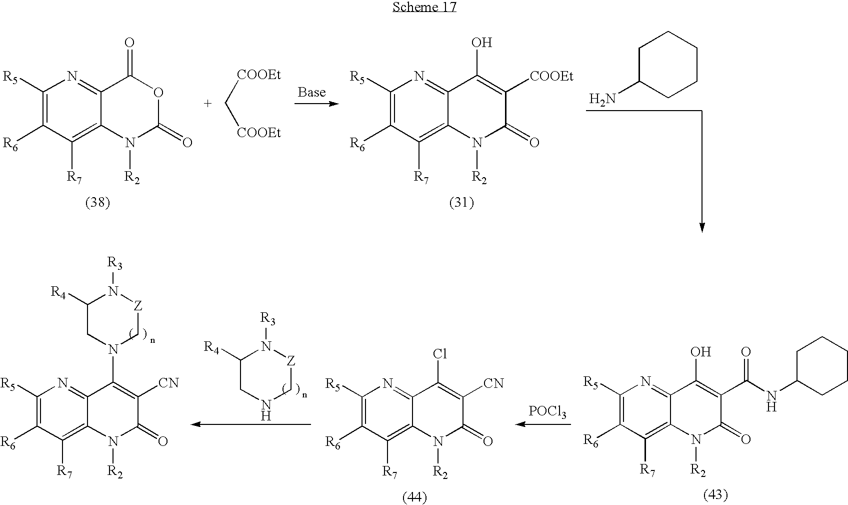

### Preparation of Compounds of General Structure (VI)

A preferred intermediate in the preparation of compound of structure (VI):

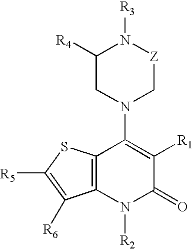

is appropriately substituted or unsubstituted 7-chloro-5-oxo-4,5-dihydro-thieno[3,2-b]pyridine-6-carboxylic acid ethyl ester, depicted by formula (47) below. To prepare this intermediate, appropriately substituted or unsubstituted methyl-3-amino-thiophene-2-carboxylate was reacted with malonyl chloride derivatives to yield intermediate 3-(2-ethoxycarbonyl-acetylamino)-thiophene-2-carboxylic acid methyl ester derivatives, depicted by formula (44). This intermediate was converted to appropriately substituted or unsubstituted 7-hydroxy-5-oxo-4,5-dihydro-thieno[3,2-b]pyridine-6-carboxylic acid ethyl ester, depicted by formula (45), by reacting with sodium ethoxide, and was then converted into appropriately substituted or unsubstituted 5,7-dichloro-thieno[3,2-b]pyridine-6-carboxylic acid ethyl ester, depicted by formula (46). Hydrolysis of this intermediate yielded appropriately substituted or unsubstituted 7-chloro-5-oxo-4,5-dihydro-thieno[3,2-b]pyridine-6-carboxylic acid ethyl ester, depicted by formula (47) as shown in Scheme 18.

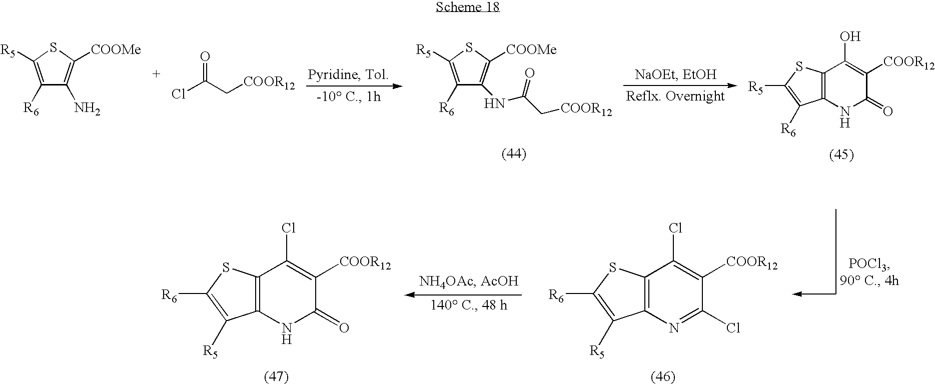

In one method, appropriately substituted or unsubstituted 7-chloro-5-oxo-4,5-dihydro-thieno[3,2-b]pyridine-6-carboxylic acid ethyl ester, depicted by formula (47), was reacted with tert-butyl-1-piperazine carboxylate to yield appropriately substituted or unsubstituted 7-(4-tert-butoxycarbonyl-piperazin-1-yl)-5-oxo-4,5-dihydro-thieno[2,3-b]-pyridine-6-carboxylic acid ethyl ester, depicted by formula (48). This intermediate was either reacted with an appropriate halide (R₂—X) or boronic acid (R₂—B(OH)₂) to yield the intermediate of structure (49), which was deprotected and reacted with an appropriate halides (R₃—X) or acid chloride (R₃—COCl) to yield target compounds of structure (VI) as shown in Scheme 19.

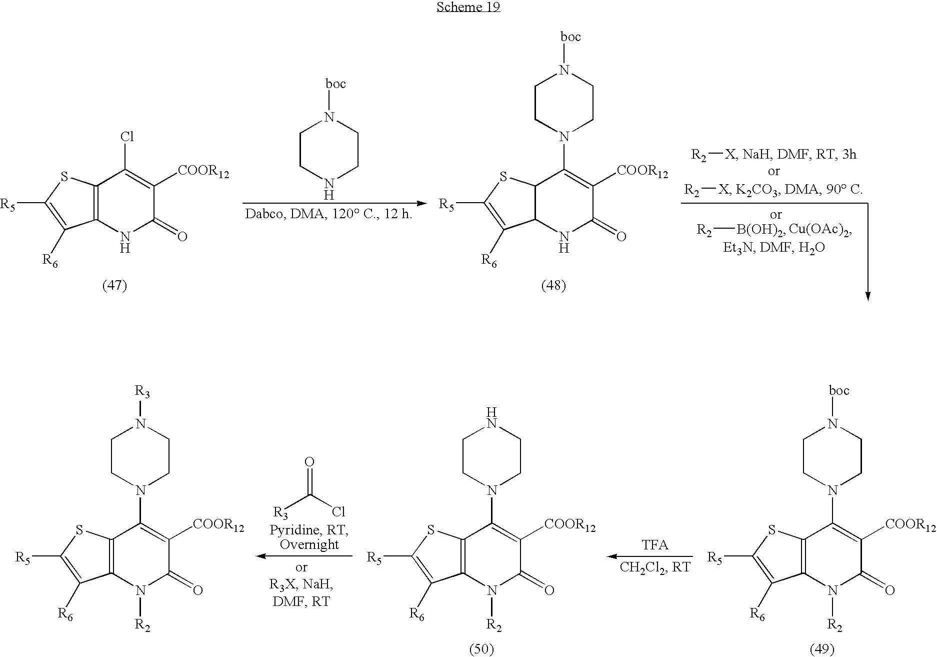

In an alternative method for the preparation of compounds of structure (VI), appropriately substituted or unsubstituted 1H-thieno[3,2-d][1,3]oxazine-2,4-dione, depicted by formula (51) in Scheme 20, was used as key intermediate. To prepare this intermediate, appropriately substituted or unsubstituted 3-amino-thiophene-2-carboxylic acid ester was hydrolyzed to corresponding 3-amino-thiophene-2-carboxylic acid which was reacted with trichloromethyl chloroformate as shown in Scheme 20. Appropriate N-substitution was introduced by reacting appropriately substituted or unsubstituted 1H-thieno[3,2-d][1,3]oxazine-2,4-dione, depicted by formula (51), with corresponding halide (R₂—X). The N-substituted or unsubstituted intermediate, depicted by formula (52), was then reacted with compound having active methylene group to get intermediate of general formula (53) which was chlorinated either by phosphorous oxychloride or by oxalyl chloride as shown in Scheme 20.

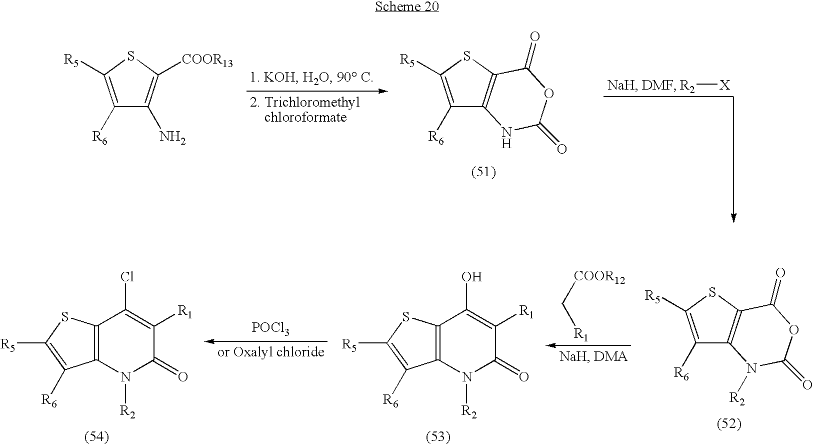

In one method, the chloro intermediate, depicted by formula (54), was reacted with piperazine to get piperazine intermediate, depicted by formula (55), as shown in Scheme 21. The piperazine intermediate, depicted by formula (55) was then reacted either with appropriate halides (R₃—X) or acid chloride (R₃—CO—Cl) to get the compound of general formula (VI), as shown in Scheme 21. Alternatively, the chloro intermediate was also reacted directly with substituted or unsubstituted piperazine to get the compound of general formula (VI), as shown in Scheme 21.

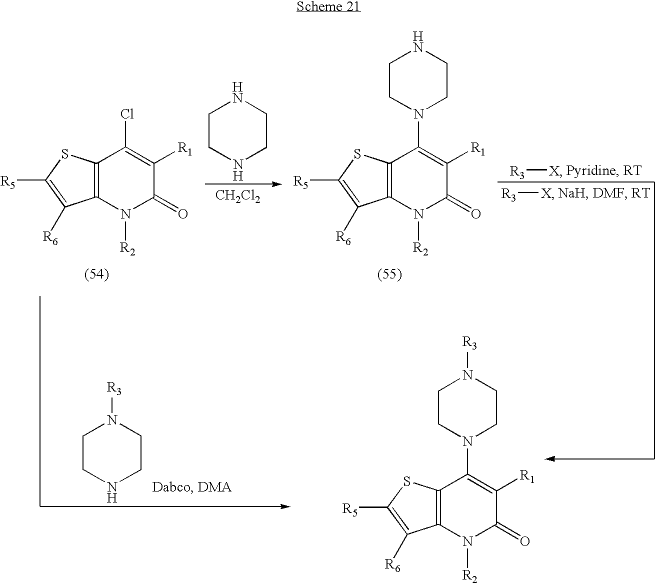

To prepare compounds of structure (VI) with R₁ as carbonitrile, appropriately substituted or unsubstituted 7-chloro-5-oxo-4,5-dihydro-thieno[3,2-b]pyridine-6-carbonitrile, depicted by formula (58), was used as a key intermediate. To prepare this intermediate, appropriately substituted or unsubstituted methyl-3-amino-thiophene-2-carboxylate was reacted with methylcyanoacetate to yield intermediate substituted or unsubstituted or unsubstituted or unsubstituted 3-(2-cyano-acetylamino)-thiophene-2-carboxylic acid methyl ester, depicted by formula (56). This intermediate was converted to appropriately substituted or unsubstituted 7-hydroxy-5-oxo-4,5-dihydro-thieno[3,2-b]pyridine-6-carbonitrile, depicted by formula (57), by reacting with sodium ethoxide, and was then converted into appropriately substituted or unsubstituted 7-chloro-5-oxo-4,5-dihydro-thieno[3,2-b]pyridine-6-carbonitrile, depicted by formula (58), as shown in Scheme 22.

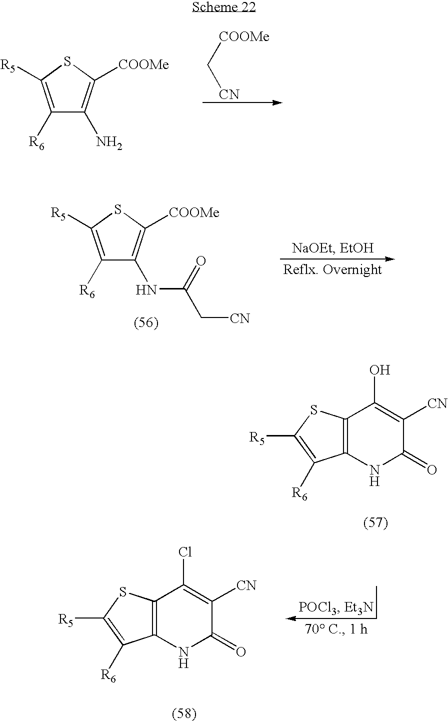

The intermediate appropriately substituted or unsubstituted 7-chloro-5-oxo-4,5-dihydro-thieno[3,2-b]pyridine-6-carbonitrile, depicted by formula (58) was reacted with tert-butyl-1-piperazine carboxylate to yield appropriately substituted or unsubstituted 7-(4-tert-butoxycarbonyl-piperazin-1-yl)-5-oxo-4,5-dihydro-thieno[2,3-b]-pyridine-6-carbonitrile, depicted by formula (59). This intermediate was either reacted with an appropriate halide (R₂—X) or boronic acid (R₂—B(OH)₂) to yield an intermediate of structure (60), which was deprotected and reacted with an appropriate acid chloride (R₃—COCl) or halide (R₃—X) to yield target compounds of structure (VI) with R₂ as carbonitrile, as shown in Scheme 23.

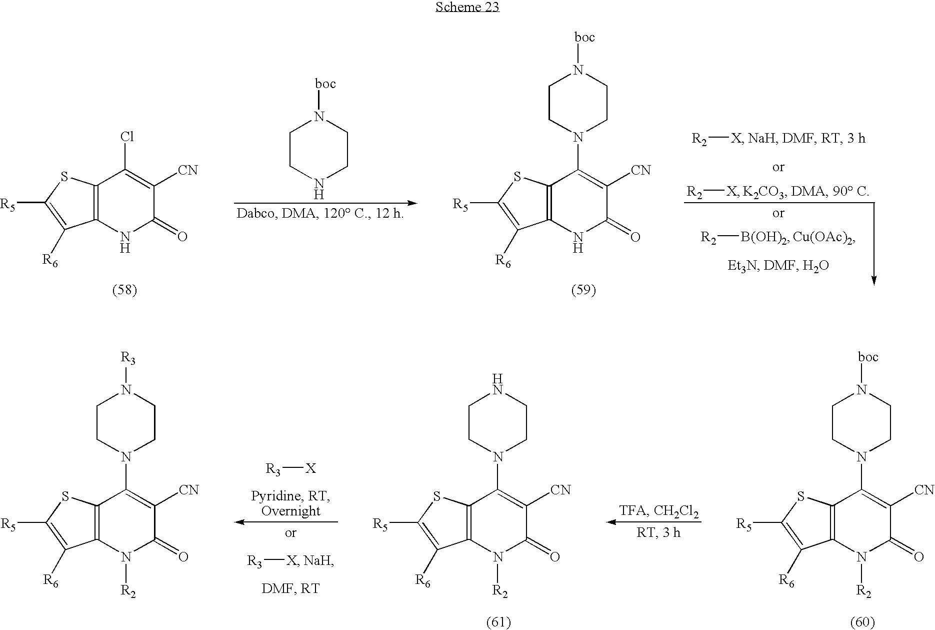

### Preparation of Compounds of Structure (VII)

Compounds of structure (VII):

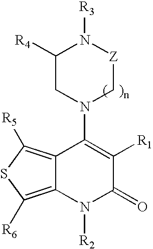

were prepared by using 4-amino-thiophene-3-carboxylic acid methyl ester, as depicted by formula (65), as a starting material. To make this compound, methyl thioglycolate was reacted with methyl acrylate to yield intermediate 3-methoxycarbonylmethylsulfanyl-propionic acid methyl ester, depicted by formula (62), which was cyclized to 4-oxo-tetrahydro-thiophene-3-carboxylic acid methyl ester, depicted by formula (63). This intermediate was reacted with hydroxylamine hydrochloride to yield 4-hydroxyimino-tetrahydro-thiophene-3-carboxylic acid methyl ester, depicted by formula (64), which yielded 4-amino-thiophene-3-carboxylic acid methyl ester hydrochloride, depicted by formula (65), as shown in Scheme 24.

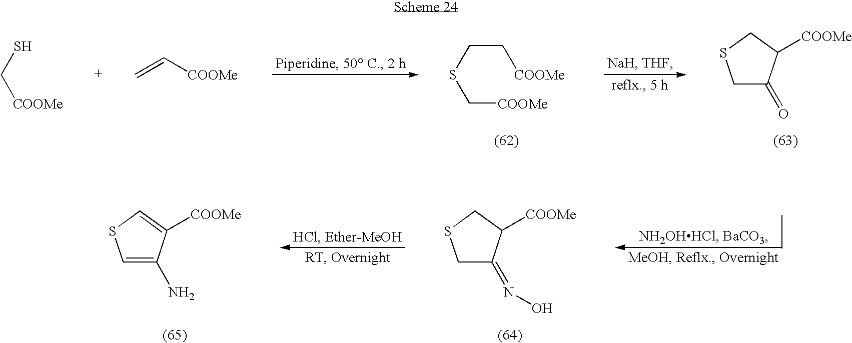

A preferred intermediate in the preparation of a compound of formula (VII) is 4-chloro-1,2-dihydro-2-oxo-thieno[3,4-b]pyridine-3-carboxylic acid ethyl ester, depicted by formula (69) below. To prepare this intermediate, 4-amino-thiophene-3-carboxylic acid methyl ester hydrochloride, depicted by formula (65), was reacted with ethylmalonyl chloride to yield intermediate 4-(2-ethoxycarbonyl-acetylamino)-thiophene-3-carboxylic acid methyl ester, depicted by formula (66). This intermediate was converted to 7-hydroxy-5-oxo-4,5-dihydro-2-thia-4-aza-indene-6-carboxylic acid ethyl ester, depicted by formula (67) by reacting with sodium ethoxide, and was then converted into 5,7-dichloro-2-thia-4-aza-indene-6-carboxylic acid ethyl ester, depicted by formula (68). Hydrolysis 5,7-dichloro-2-thia-4-aza-indene-6-carboxylic acid ethyl ester, depicted by formula (68), yielded 7-chloro-5-oxo-4,5-dihydro-2-thia-4-aza-indene-3-carboxylic acid ethyl ester, depicted by formula (69), as shown in Scheme 25.

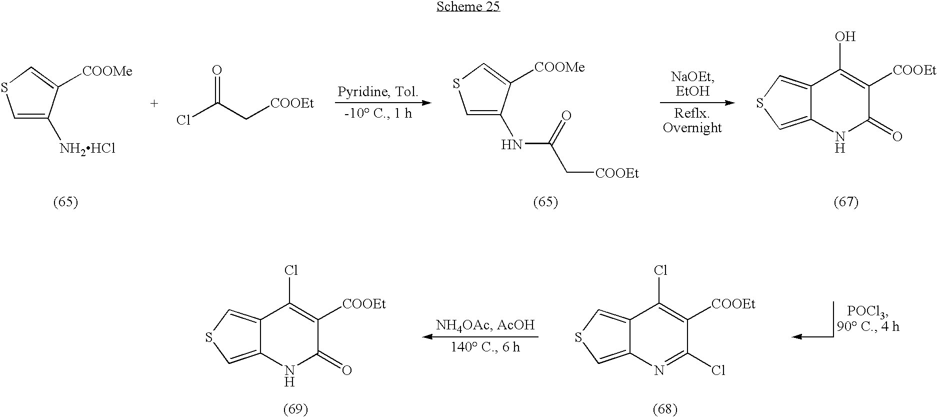

In one method, intermediate 7-chloro-5-oxo-4,5-dihydro-2-thia-4-aza-indene-3-carboxylic acid ethyl ester, depicted by formula (69), was reacted with tert-butyl-1-piperazine carboxylate to yield 7-(4-tert-butoxycarbonyl-piperazin-1-yl)-5-oxo-4,5-dihydro-2-thia-4-aza-indene-6-carboxylic acid ethyl ester, depicted by formula (70). This intermediate was either reacted with an appropriate halide (R₂—X) or boronic acid (R₂—B(OH)₂) to yield an intermediate of structure (71), which was deprotected and reacted with an appropriate acid chloride (R₃—COCl) or halide (R₃—X) to yield target compounds of structure (VII), with R₁ as ethyl carboxylate, R₂ and R₃ as defined above, as shown in Scheme 26.

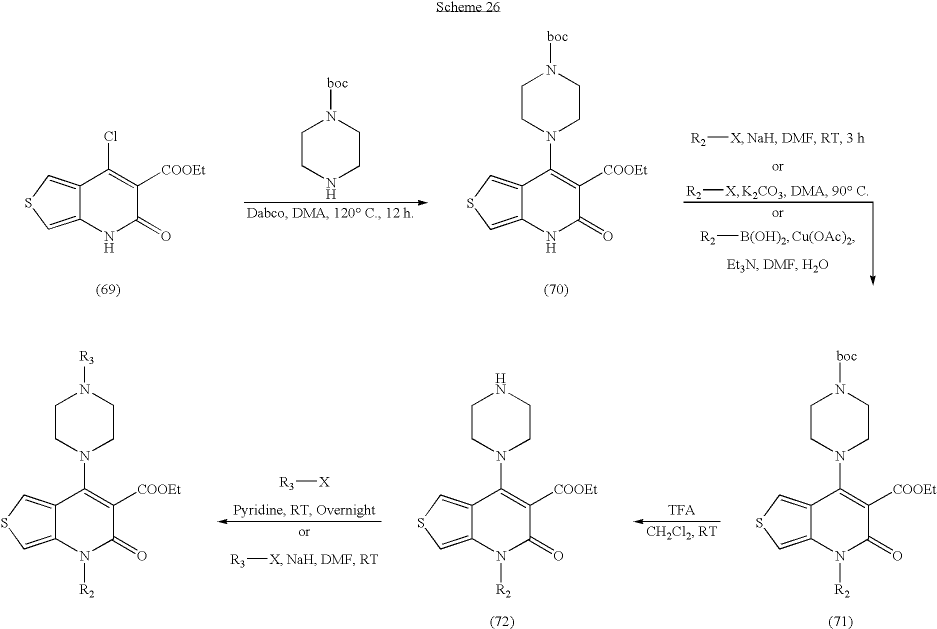

To prepare compounds of structure (VII) with R₁ as carbonitrile, and R₂ and R₃ as defined above, 7-chloro-5-oxo-4,5-dihydro-2-thia-4-aza-indene-6-carbonitrile, depicted by formula (75), was used as a key intermediate. To prepare this intermediate, 7-hydroxy-5-oxo-4,5-dihydro-2-thia-4-aza-indene-6-carboxylic acid ethyl ester, depicted by formula (67), was reacted with cyclohexylamine to yield intermediate 7-hydroxy-5-oxo-4,5-dihydro-2-thia-4-aza-indene-6-carboxylic acid cyclohexylamide, depicted by formula (73). This intermediate was converted to 5,7-dichloro-2-thia-4-aza-indene-6-carbonitrile, depicted by formula (74), by reacting with phosphorous oxychloride, and was then converted into 7-chloro-5-oxo-4,5-dihydro-2-thia-4-aza-6-carbonitrile, depicted by formula (75), as shown in Scheme 27.

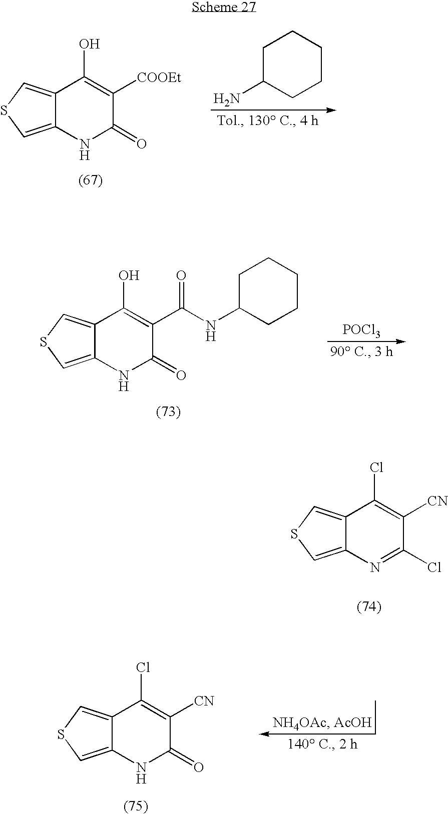

Compounds of structure (VII) with R₁ as carbonitrile, and R₂ and R₃ as defined above were also prepared from intermediate 7-chloro-5-oxo-4,5-dihydro-2-thia-4-aza-indene-6-carbonitrile, depicted by formula (75). Intermediate 7-chloro-5-oxo-4,5-dihydro-2-thia-4-aza-indene-6-carbonitrile, depicted by formula (75), was reacted with tert-butyl-1-piperazine carboxylate to yield 7-(4-tert-butoxycarbonyl-piperazin-1-yl)-5-oxo-4,5-dihydro-2-thia-4-aza-indene-6-carbonitrile, depicted by formula (76). This intermediate was either reacted with an appropriate halide (R₂—X) or boronic acid (R₂—B(OH)₂) to yield intermediate of structure (77), which was deprotected and reacted with an appropriate acid chloride (R₃—COCl) or halide (R₃—X) to yield target compounds of structure (VII) with R₁ as carbonitrile, and R₂ and R₃ as defined above, as shown in Scheme 28.

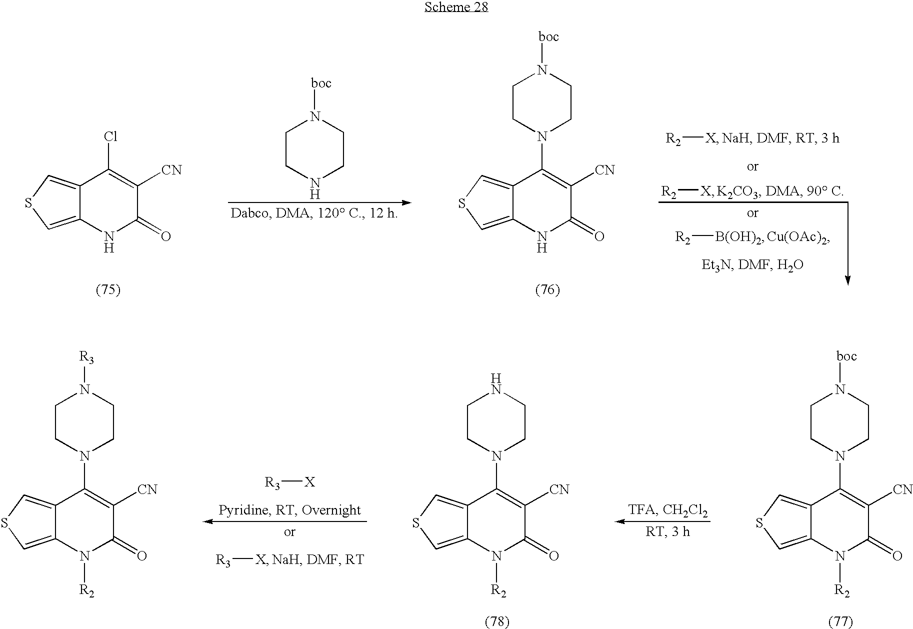

### Preparation of Compounds of Structure (VIII)

A preferred intermediate in the preparation of compounds of structure (VIII):

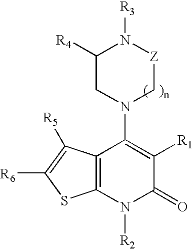

is appropriately substituted or unsubstituted 4-chloro-6-oxo-6,7-dihydro-thieno[2,3-b]pyridine-5-carboxylic acid ethyl ester, depicted by formula (82) below. To prepare this intermediate, appropriately substituted or unsubstituted methyl-2-amino-thiophene-3-carboxylate was reacted with ethylmalonyl chloride to yield appropriately substituted 2-(2-ethoxycarbonyl-acetylamino)-thiophene-3-carboxylic acid methyl ester, depicted by formula (79). This intermediate was converted to appropriately substituted 4-hydroxy-6-oxo-6,7-dihydro-thieno[2,3-b]pyridine-5-carboxylic acid ethyl ester, depicted by formula (80), by reacting it with sodium ethoxide, and then converting it into appropriately substituted 4,6-dichloro-thieno[2,3-b]pyridine-5-carboxylic acid ethyl ester, depicted by formula (81). Hydrolysis of appropriately substituted 4,6-dichloro-thieno[2,3-b]pyridine-5-carboxylic acid ethyl ester, depicted by formula (81), yielded appropriately substituted 4-chloro-6-oxo-6,7-dihydro-thieno[2,3-b]pyridine-5-carboxylic acid ethyl ester, depicted by formula (82), as shown in Scheme 29.

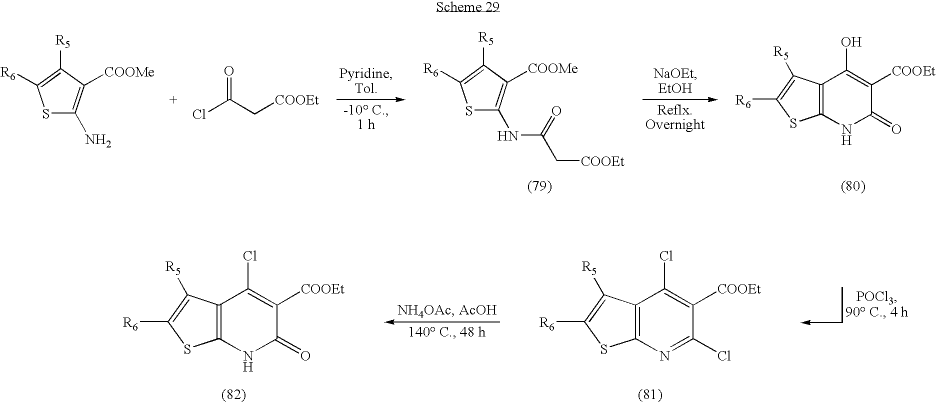

Appropriately substituted or unsubstituted 4-chloro-6-oxo-6,7-dihydro-thieno[2,3-b]pyridine-5-carboxylic acid ethyl ester intermediate, depicted by formula (82), was reacted with tert-butyl-1-piperazine carboxylate to yield appropriately substituted or unsubstituted 4-(4-tert-butoxycarbonyl-piperazin-1-yl)-6-oxo-6,7-dihydro-thieno[2,3-b]-pyridine-5-carboxylic acid ethyl ester, depicted by formula (83). This intermediate was either reacted with an appropriate halide (R₂—X) or boronic acid (R₂—B(OH)₂) to yield intermediate of structure (84), which was deprotected and reacted with an appropriate acid chloride (R₃—COCl) or halide (R₃—X) to yield target compounds of structure (VIII) with R₁ as ethyl carboxylate, and R₂ and R₃ as defined above, as shown in Scheme 30.

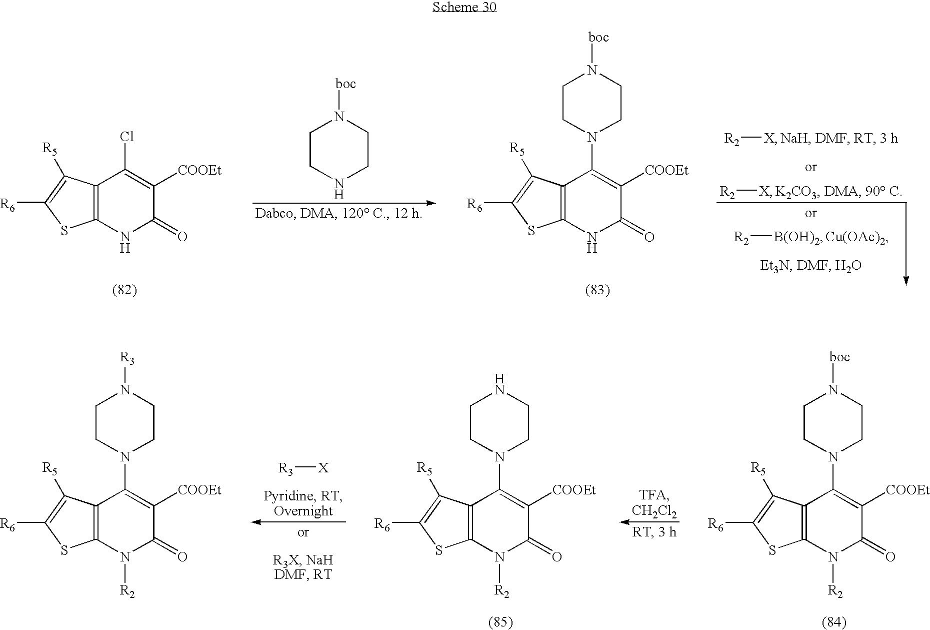

To yield compounds of structure (VIII) with R₁ as carbonitrile, and R₂ and R₃ as defined above, appropriately substituted or unsubstituted 4-chloro-6-oxo-6,7-dihydro-thieno[2,3-b]pyridine-5-carbonitrile, depicted by formula (88), was used as a key intermediate. To prepare this intermediate, appropriately substituted or unsubstituted 4-hydroxy-6-oxo-6,7-dihydro-thieno[2,3-b]pyridine-5-carboxylic acid ethyl ester, depicted by formula (80), was reacted with cyclohexylamine to yield appropriately substituted or unsubstituted 4-hydroxy-6-oxo-6,7-dihydro-thieno[2,3-b]pyridine-5-carboxylic acid cyclohexylamide intermediate, depicted by formula (86). This intermediate was converted to appropriately substituted or unsubstituted 4,6-dichloro-thieno[2,3-b]pyridine-5-carbonitrile, depicted by formula (87), by reacting with phosphorous oxychloride, which was then converted into appropriately substituted or unsubstituted 4-chloro-6-oxo-6,7-dihydro-2-thieno[2,3-b]pyridine-5-carbonitrile, depicted by formula (88), as shown in Scheme 31.

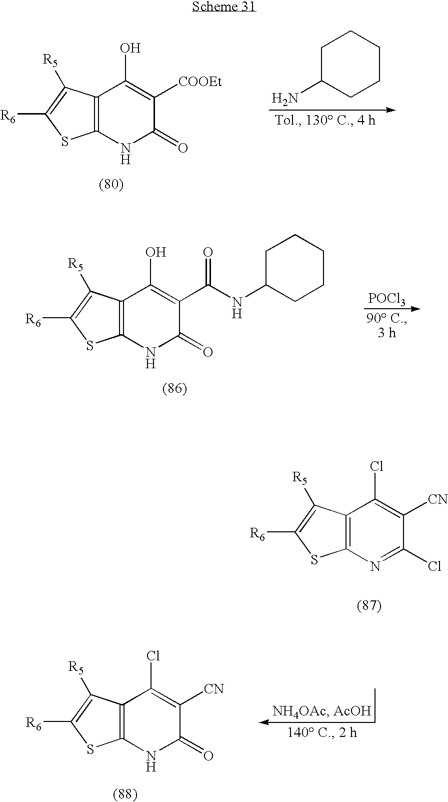

Compounds of structure (VIII) with R₁ as carbonitrile, and R₂ and R₃ as defined above were prepared from appropriately substituted or unsubstituted 4-chloro-6-oxo-6,7-dihydro-thieno[2,3-b]pyridine-5-carbonitrile intermediate, depicted by formula (88). Intermediate 4-chloro-6-oxo-6,7-dihydro-thieno[2,3-b]pyridine-5-carbonitrile, depicted by formula (88), was reacted with tert-butyl-1-piperazine carboxylate to yield 4-(5-cyano-6-oxo-6,7-dihydro-thieno[2,3-b]-pyridine-4-yl)-piperazine-1-carboxylic acid tert-butyl ester, depicted by formula (89). This intermediate was either reacted with an appropriate halide (R₂—X) or boronic acid (R₂—B(OH)₂) to yield an intermediate of structure (90), which was deprotected and reacted with an appropriate acid chloride (R₃—COCl) or halide (R₃—X) to yield target compounds of structure (VIII) with R₁ as carbonitrile, and R₂ and R₃ as defined above, as shown in Scheme 32.

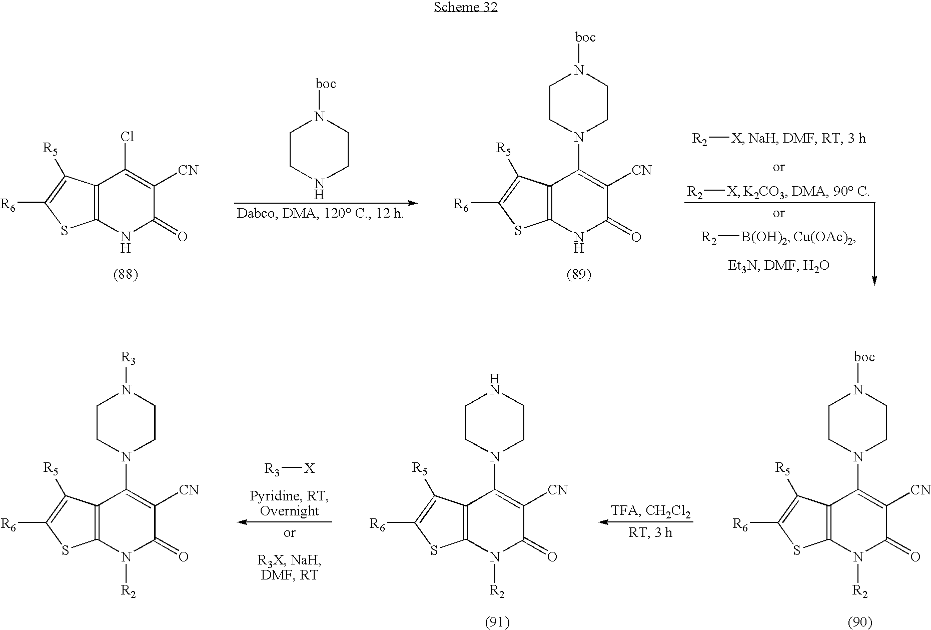

In an alternative method for the preparation of compounds of structure (VIII), appropriately substituted or unsubstituted 1H-thieno[2,3-d][1,3]oxazine-2,4-dione, depicted by formula (92) in Scheme 33, was used as key intermediate. To prepare this intermediate, appropriately substituted or unsubstituted 2-amino-thiophene-3-carboxylic acid ester was hydrolyzed to corresponding 2-amino-thiophene-3-carboxylic acid which was reacted with trichloromethyl chloroformate as shown in Scheme 33. Appropriate N-substitution was introduced by reacting s appropriately substituted or unsubstituted 1H-thieno[2,3-d][1,3]oxazine-2,4-dione, depicted by formula (92), with corresponding halide (R₁—X). N-substituted or unsubstituted intermediate, depicted by formula (93) was then reacted with compounds having active methylene group to get intermediate of general formula (94) which was chlorinated either by phosphorous oxychloride or by oxalyl chloride.

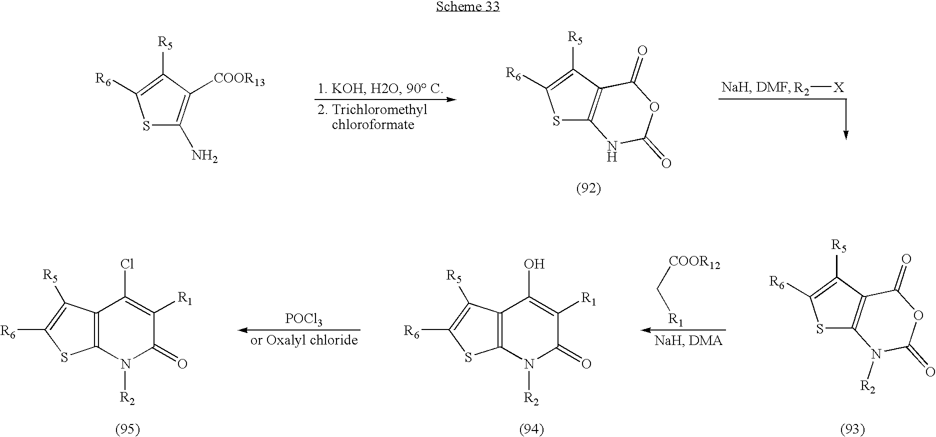

In one method, the chloro intermediate, depicted by formula (95) was reacted with piperazine to get piperazine intermediate, depicted by formula (96), as shown in Scheme 34. The piperazine intermediate (96) was then reacted either with acid chloride (R₃—CO—Cl) or with appropriate halide (R₃—X) gave the compound of general formula (VIII) as shown in Scheme 34. Alternatively, the chloro intermediate was also reacted with substituted or unsubstituted piperazine to get compound of general formula (VIII), as shown in Scheme 34.

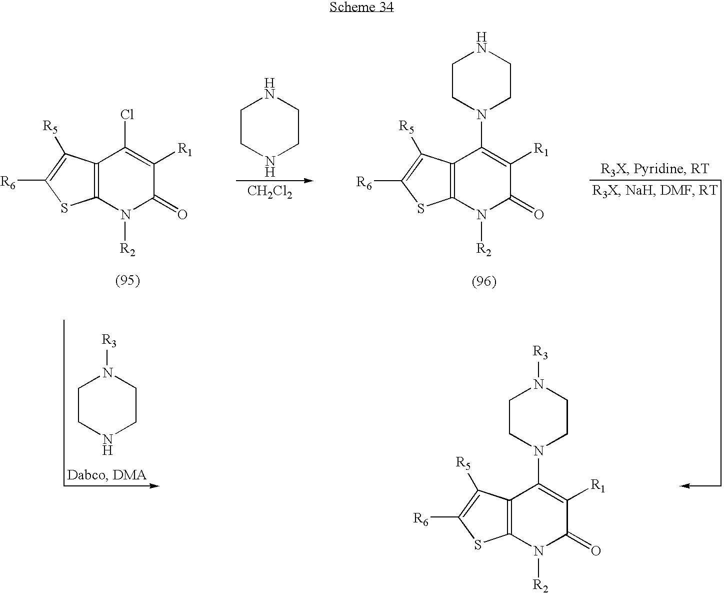

The inhibitors of TNF-α of preferred embodiments were prepared by the methods described in following examples.

### Preparation of 6-methyl-1H-benzo[d][1,3]oxazine-2,4-dione

Trichloromethyl chloroformate (144.32 g, 729 mmol) was added slowly to a solution of 2-amino-5-methyl benzoic acid (100 g, 661 mmol) in anhydrous dioxane (500 mL) stirred at room temperature under argon. While adding trichloromethyl chloroformate, the temperature of solution was maintained at room temperature. After completion of addition, the solution was heated at 110° C. for 2 h. The solution was cooled to room temperature and diluted by equal amount of isopropyl ether. The solution was allowed to stand overnight at room temperature. The solids formed were filtered, washed by diethyl ether and dried under vacuum Yield 109 g (93%). M.P. 257° C. ¹H NMR (DMSO-d₆): δ 2.32 (s, 3H), 7.06 (d, J=8.6 Hz, 1H), 7.56 (dd, J=8.6, 1.8 Hz, 1H), 7.71 (d, J=1.1, 1H), 11.63 (s, 1H). EIMS (neg. mode) m/z 176 (M−1), 152 (M+23). Anal. (C₉H₇NO₃) C, H, N.

### Preparation of 6-methyl-1-pyridin-2-ylmethyl-1H-benzo[d][1,3]oxazine-2,4-dione

Solid 6-methyl-1H-benzo[d][1,3]oxazine-2,4-dione (35.83 g, 202 mmol) was added to a suspension of NaH (60% in Min. oil, 18.2 g, 455 mmol) in dry DMF (350 mL) stirred at −40° C. under argon. The solution was allowed to come to room temperature by removing the dry ice bath. After stirring at room temperature briefly, the solution was again cooled to −20° C. Then 2-bromomethylpyridine hydrobromide (55 g, 217 mmol) was added to the solution. The solution was allowed to come to room temperature and stirred overnight at the same temperature under argon. The solution was poured into ice cold 5% aqueous NaCl solution (5 L). The solids formed were filtered and air dried at room temperature over night. The dry solids were suspended in hexanes (1250 mL) and stirred vigorously at room temperature for 1 h. The solids were filtered and dried under vacuum. Yield 45 g (83%). ¹H-NMR (DMSO-d₆): δ 2.33 (s, 3H), 5.34 (s, 2H), 7.17 (d, J=8.8 Hz, 1H), 7.30 (m, 1H), 7.48 (d, J=8.0 Hz, 1H), 7.55 (dd, J=2.0, 8.8 Hz, 1H), 7.78 (m, 1H), 7.85 (d, J=1.6 Hz, 1H), 8.50 (d, J=4.4 Hz, 1H); EIMS m/z 269 (M+1).

### Preparation of 4-chloro-6-methyl-2-oxo-1-pyridin-2-ylmethyl-1,2-dihydro-quinoline-3-carboxylic acid ethyl ester

Neat diethylmalonate (26.74 mL, 176 mmol) was added to a suspension of NaH (60% in min. oil, 7.72 g, 193 mmol) in dry DMF (340 mL) stirred at −50° C. under argon. The solution was stirred at this temperature for 5 min and then allowed to come to room temperature slowly by removing the dry ice bath. The solution was stirred at room temperature until the evolution of gas ceased. Solid 6-methyl-1-pyridin-2-ylmethyl-1H-benzo[d][1,3]oxazine-2,4-dione (45 g, 167 mmol) was added to the solution at once. Then the reaction was heated to 120° C. The solution was further stirred at 120° C. for 1 h. The solution was again cooled to −50° C. and neat oxalyl chloride (44 mL, 501 mmol) was added slowly and carefully via a syringe. The solution was allowed to come to room temperature by removing the dry ice bath. The solution was then heated to 100° C. for 5 h (LC/MS controlled). The solution was cooled to 5° C. and poured into ice water (4.5 L), basified by adding 4M aq. NaOH solution (ca. 75 mL) and stirred for 15 min. The solids were filtered, washed by cold water and dried under vacuum. Yield 52 g (87%). ¹H-NMR (DMSO-d₆): δ 1.31 (t, J=7.2 Hz, 1H), 2.40 (s, 3H), 4.37 (q, J=7.2 Hz, 2H), 5.61 (s, 2H), 7.25 (m, 2H), 7.43 (d, J=8.8 Hz, 1H), 7.53 (dd, J=8.8, 4.0 Hz, 1H), 7.75 (m, TH), 7.83 (d, J=0.8 Hz, 1H), 8.44 (dd, J=4.0, 0.8 Hz, 1H); EIMS m/z 357 (M+1).

### Preparation of 6-Methyl-4-(4-methyl-piperazin-1-yl)-2-oxo-1-pyridin-2-ylmethyl-1,2-dihydro-quinoline-3-carboxylic acid ethyl ester

A solution of 4-chloro-6-methyl-2-oxo-1-pyridin-2-ylmethyl-1,2-dihydro-quinoline-3-carboxylic acid ethyl ester (45 g, 126 mmol) and 1-methyl piperazine (18.2 mL, 163 mmol) in DCE (340 mL) was heated at 45° C. After 2 h, another equivalent of 1-methyl piperazine (14 mL, 126 mmol) was added. The solution was heated overnight at 85° C. The solution was cooled and diluted with aq. NaOH solution (189 mmol NaOH, 300 mL of 0.63 M solution). The solution was stirred for 15 min and the organic layer was separated. The organic layer was washed by water (2×), dried over MgSO₄ and concentrated under vacuum. The residue was dissolved in minimum CH₂Cl₂ and diluted with diethyl ether. The crystals formed were filtered out. The product contained <1% unreacted chloro starting material that was purified as follows.

The crude product (37.86 g, 90 mmol) was dissolved in CH₂Cl₂ (284 mL). The solution was diluted with a solution of NaHSO₄ (13.51 g, 112 mmol) in water (473 mL) and stirred for awhile. The aqueous phase was separated and basified with aq. NaOH solution (31.5 mL of 4M solution, 126 mmol). The solids formed were filtered, washed with cold water and dried under vacuum. Yield 34 g with purity >98%. In order to ensure maximum possible purity, the product was then purified by flash chromatography eluting with 8% MeOH in CH₂Cl₂ gradient within 190 min. with a flow rate of 70 mL/min through RediSep® SiO₂ column (300 g) by using detection wavelength of 225 nm in combiflash system (Isco). ¹H-NMR (DMSO-d₆): δ 1.29 (t, J=7.2 Hz, 1H), 2.27 (s, 3H), 2.36 (s, 3H), 2.55 (m, 4H), 3.11 (m, 4H), 4.29 (q, J=7.2 Hz, 2H), 5.51 (s, 2H), 7.15 (d, J=8 Hz, 1H), 7.26 (m, 2H), 7.34 (dd, J=8.8, 2.0 Hz, 1H), 7.65 (s, 1H), 7.76 (m, 1H), 8.46 (d, J=6.0, Hz, 1H); EIMS m/z 421 (M+1).

General Procedure for the Coupling of Chloro Compounds with Amines

General Procedure A

A solution of corresponding 4-chloro-6-substituted or unsubstituted-2-oxo-1-substituted or unsubstituted-1,2-dihydro-quinoline-3-carboxylic acid ethyl ester (1 eqv.), corresponding primary or secondary amine (1.1 eqv.) and Dabco (2 eqv.) in dry DMF was heated overnight at 110° C. The solution was cooled and excess DMF was distilled off under vacuum. The residue was dissolved in CH₂Cl₂ and washed by water. The organic phase was dried over MgSO₄ and concentrated. The crude product was purified by flash chromatography eluting with 0-5% MeOH in CH₂Cl₂ gradient.

General Procedure B

A solution of corresponding 4-chloro-6-substituted or unsubstituted-2-oxo-1-substituted or unsubstituted-1,2-dihydro-quinoline-3-carboxylic acid ethyl ester (1 eqv.), and corresponding primary or secondary amine (5 eqv.) in CH₂Cl₂ was stirred at r.t. for 12 hours. The CH₂Cl₂ was washed once with water containing 2 equiv. NaOH, then washed twice with water. The organic phase was dried over MgSO₄ and concentrated under vacuum. The crude product was purified by flash chromatography using 0-20% MeOH in CH₂Cl₂ gradient over 200 min in a combiflash.

### Preparation of 4-(4-Formyl-piperazin-1-yl)-6-methyl-2-oxo-1-pyridin-2-ylmethyl-1,2-dihydro-quinoline-3-carboxylic acid ethyl ester

This compound was prepared from 4-chloro-6-methyl-2-oxo-1-pyridin-2-ylmethyl-1,2-dihydro-quinoline-3-carboxylic acid ethyl ester and 1-piperazinecarboxyaldehyde by using general procedure A. ¹H-NMR (DMSO-d₆): δ 1.29 (t, J=7.2 Hz, 1H), 2.36 (s, 3H), 3.09 (m, 4H), 3.47 (m, 2H), 3.61 (m, 2H), 4.26 (q, J=7.2 Hz, 2H), 5.52 (s, 2H), 7.17 (d, J=7.6 Hz, 1H), 7.28 (m, 2H), 7.37 (dd, J=8.8, 1.6 Hz, 1H), 7.74 (m, 2H), 8.10 (s, 1H), 8.47 (d, J=4.8, Hz, 1H); EIMS m/z 435 (M+1). Analysis (C, N, H).

### Preparation of 2-amino-5-methyl benzoic acid

To a solution of 5-methyl-2-nitrobenzoic acid (20 g, 110 mmol) in ethanol was added 10% Pd/C (1 g). The mixture was stirred overnight at room temperature under hydrogen atmosphere. The solution was filtered through celite and evaporated under reduced pressure to yield 16 g (96%) of white solids. M.P. 162° C. ¹H NMR (DMSO-d₆): δ 2.13 (s, 3H), 6.65 (d, J=8.6 Hz, 1H), 7.06 (dd, J=8.6, 1.8 Hz, 1H), 7.48 (d, J=1.1, 1H). EIMS m/z 174 (M+1), 152 (M+23).

### Preparation of 6-Methyl-1H-benzo[d][1,3]oxazine-2,4-dione

Trichloromethyl chloroformate (36.27 mL, 300 mmol) was added to a stirred solution of Compound 10 (41.3 g, 273 mmol) in dry dioxane at room temperature and the solution was refluxed for 4 h. The solution was cooled in ice bath and the solids formed were filtered. The solids were washed by ether and dried under vacuum at room temperature to yield 45.5 g (94%) of white solids. M.P. 257° C. ¹H NMR (DMSO-d₆): δ 2.32 (s, 3H), 7.06 (d, J=8.6 Hz, 1H), 7.56 (dd, J=8.6, 1.8 Hz, 1H), 7.71 (d, J=1.1, 1H), 11.63 (s, 1H). EIMS (neg. mode) m/z 176 (M−1), 152 (M+23). Anal. (C₉H₇NO₃) C, H, N.

### Preparation of 1-Benzyl-6-methyl-1H-benzo[d][1,3]oxazine-2,4-dione

A solution of 6-methyl-1H-benzo[d][1,3]oxazine-2,4-dione (25 g, 141 mmol) in DMF was added slowly to a suspension of NaH (60% in mineral oil, 6.21 g, 155 mmol) in DMF and further stirred at room temperature for 1 h. Then, neat benzyl bromide (19.53 mL, 155 mmol) was added and the solution was further stirred at room temperature for 3 h. The solution was poured into ice water, and the solids formed were filtered, washed several times by water, and dried. The solid was suspended in hexane, sonicated briefly, filtered, and washed by hexane to yield 36.5 g (97%) of white solids. M.P. 150° C.

¹H NMR (DMSO-d₆): δ 2.32 (s, 3H), 5.27 (s, 2H), 7.15 (d, J=8.7 Hz, 1H), 7.26-7.39 (m, 5H), 7.54 (dd, J=1.5, 8.7 Hz, 1H), 7.83 (d, J=1.5 Hz, 1H). Anal. (C₁₆H₁₃NO₃) C, H, N.

### Preparation of 1-Benzyl-4-hydro-6-methyl-2-oxo-1,2-dihydro-quinoline-3-carboxylic acid ethyl ester

Neat diethyl malonate (19.07 mL, 125 mmol) was added slowly to a suspension of sodium hydride (60% in mineral oil, 5.52 g, 138 mmol) in dimethylacetamide under N₂ atmosphere. The mixture was stirred at room temperature until the evolution of hydrogen gas ceased, then heated to 90° C. for 30 min and cooled to room temperature. A solution of 1-benzyl-6-methyl-1H-benzo[d][1,3]oxazine-2,4-dione (36.8 g, 138 mmol) in dimethylacetamide was added slowly and the mixture was heated overnight at 110° C. The mixture was cooled to room temperature, poured into ice water, and acidified by cold 10% HCl. The solids formed were filtered, washed several times by water, and dried at room temperature under vacuum to yield 41 g (97%) of white solids. M.P. 113° C. ¹H NMR (DMSO-d₆): δ 1.31 (t, J=7.5 Hz, 3H), 2.33 (s, 3H), 4.35 (q, J=7.5 Hz, 2H), 5.43 (s, 2H), 7.15-7.30 (m, 6H), 7.43 (dd, J=1.6, 8.7 Hz, 1H), 7.85 (d, J=1.5 Hz, 1H). EIMS m/z 338 (M+T), 360 (M+23). Anal. (C₂₀H₁₉NO₄) C, H, N.

### Preparation of 1-benzyl-4-chloro-6-methyl-2-oxo-1,2-dihydro-quinoline-3-carboxylic acid ethyl ester

A solution of 1-benzyl-4-hydroxy-6-methyl-2-oxo-1,2-dihydro-quinoline-3-carboxylic acid ethyl ester (30 g, 89 mmol) in 100 mL neat phosphorus oxychloride was heated at 100° C. for 3 h. The solvent was evaporated under reduced pressure. The residue was suspended in ice water and neutralized by solid sodium bicarbonate. The solids formed were filtered, washed by water, and purified by flash chromatography eluting with 1% methanol in dichloromethane to yield 14.6 g (46%) of white solids. M.P. 103° C. ¹H NMR (DMSO-d₆): δ 1.34 (t, J=5.6 Hz, 3H), 2.39 (s, 3H), 4.37 (q, J=5.6 Hz, 2H), 5.53 (s, 2H), 7.18 (d, J=6.4 Hz, 1H), 7.24 (m, 1H), 7.30 (m, 2H), 7.43 (d, J=7.2 Hz, 1H), 7.53 (dd, J=1.2, 6.8 Hz, 1H), 7.83 (s, 1H).

### Preparation of 4-(4-formyl-piperazin-1-yl)-6-methyl-2-oxo-1-phenyl-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. 240° C. ¹H-NMR (DMSO-d₆): δ 1.27 (t, J=7.2 Hz, 3H), 2.39 (s, 3H), 3.14 (m, 4H), 3.64 (m, 4H), 4.27 (q, J=7.2 Hz, 2H), 6.45 (d, J=8.4 Hz, 1H), 7.30 (m, 3H), 7.5-7.6 (m, 3H), 7.76 (s, 1H), 8.12 (s, 1H); EIMS m/z 420 (M+1).

### Preparation of 1-benzyl-4-dimethylamino-6-methyl-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. 96-103° C. ¹H-NMR (DMSO-d₆): δ 1.30 (t, J=7.2 Hz, 3H), 2.35 (s, 3H), 2.92 (s, 6H), 4.28 (q, J=7.2 Hz, 2H), 5.44 (s, 2H), 7.15-7.35 (m, 7H), 7.70 (s, 1H); EIMS m/z 365 (M+1).

### Preparation of 1-benzyl-4-[(2-dimethylamino-ethyl)-methyl-amino]-6-methyl-2-oxo-1,2-dihydro-quinoline-3-carboxylic acid ethyl ester hydrochloride salt

This compound was prepared from 1-benzyl-4-chloro-6-methyl-2-oxo-1,2-dihydro-quinoline-3-carboxylic acid ethyl ester by using general procedure A. ¹H-NMR (DMSO-d₆): δ 1.33 (t, J=7.2 Hz, 3H), 2.39 (s, 3H), 2.73 (s, 6H), 2.91 (s, 3H), 3.46 (m, 2H), 4.33 (q, J=7.2 Hz, 2H), 5.46 (b, 2H), 7.15-7.35 (m, 6H), 7.38 (dd, J=2.0, 8.8 Hz, 1H), 7.88 (s, 1H); EIMS m/z 422 (M+1).

### Preparation of 1-benzyl-4-(4-dimethylamino-piperidin-1-yl)-6-methyl-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. >60° C. ¹H-NMR (DMSO-d₆): δ 1.30 (t, J=7.2 Hz, 3H), 1.63 (m, 2H), 1.89 (m, 2H), 2.25 (m, 7H), 2.37 (s, 3H), 2.87 (m, 2H), 4.29 (q, J=7.2 Hz, 2H), 5.44 (b, 2H), 7.15-7.35 (m, 7H), 7.61 (s, 1H); EIMS m/z 448 (M+1).

### Preparation of 1-benzyl-4-(2,5-diaza-bicyclo[2.2.1]hept-2-yl)-6-methyl-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. >72° C. ¹H-NMR (DMSO-d₆): δ 1.28 (t, J=6.8 Hz, 3H), 1.66 (d, J=9.2 Hz, 1H), 1.85 (d, J=9.2 Hz, 1H), 2.33 (s, 3H), 2.90 (d, J=10.0 Hz, 1H), 3.16 (d, J=10.0 Hz, 1H), 3.64 (m, 2H), 4.23 (q, J=6.8 Hz, 2H), 4.43 (s, 1H), 5.38 (b, 2H), 7.15-7.35 (m, 7H), 7.70 (s, 1H); EIMS m/z 418 (M+1).

### Preparation of 1-benzyl-4-[1,4]diazepan-1-yl-6-methyl-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. >55. ¹H-NMR (DMSO-d₆): δ 1.31 (t, J=7.2 Hz, 3H), 1.81 (m, 2H), 2.36 (s, 3H), 2.90 (m, 4H), 3.23 (m, 5H), 4.29 (q, J=7.2 Hz, 2H), 5.45 (s, 2H), 7.15-7.40 (m, 7H), 7.80 (m, 1H); EIMS m/z 420 (M+1).

### Preparation of 1-benzyl-4-(4-formyl-piperazin-1-yl)-6-methyl-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. 196° C. ¹H-NMR (DMSO-d₆): δ 1.30 (t, J=7.2 Hz, 3H), 2.38 (s, 3H), 3.10 (m, 4H), 3.62 (m, 4H), 4.31 (q, J=7.2 Hz, 2H), 5.46 (s, 2H), 7.10-7.40 (m, 7H), 7.75 (s, 1H), 8.11 (s, 1H); EIMS m/z 434 (M+1).

### Preparation of 1-benzyl-4-(3-dimethylamino-pyrrolidin-1-yl)-6-methyl-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P.>40° C. ¹H-NMR (DMSO-d₆): δ 1.29 (t, J=7.2 Hz, 3H), 1.76 (m, 1H), 2.15 (m, 7H), 2.34 (s, 3H), 2.79 (m, 1H), 3.40 (m, 2H), 3.65 (m, 1H), 4.25 (q, J=7.2 Hz, 2H), 5.40 (m, 2H), 7.14 (m, 2H), 7.23 (m, 2H), 7.30 (m, 3H), 7.74 (s, 1H); EIMS m/z 434 (M+1).

### Preparation of 1-benzyl-6-methyl-4-(4-methyl-[4] diazepan-1-yl)-2-oxo-1,2-dihydro-quinoline-3-carboxylic acid ethyl ester

This compound was prepared from 1-benzyl-4-chloro-6-methyl-2-oxo-1,2-dihydro-quinoline-3-carboxylic acid ethyl ester by using general procedure A. M.P. 120° C. ¹H-NMR (DMSO-d₆): δ 1.31 (t, J=7.2 Hz, 3H), 1.91 (m, 2H), 2.33 (s, 3H), 2.36 (s, 3H), 2.68 (m, 4H), 4.30 (q, J=7.2 Hz, 2H), 5.45 (s, 2H), 7.16 (m, 2H), 7.23 (m, 1H), 7.33 (m, 4H), 7.75 (s, 1H); EIMS m/z 434 (M+1).

### Preparation of 5-fluoro-2-nitrobenzoic acid

3-Fluorobenzoic acid (1 g, 7.13 mmol) was dissolved in concentrated H₂SO₄ (2 ml) by warming slightly above room temperature. The solution was cooled to 0° C. Fuming nitric acid (539 mg, 8.56 mmol) was added slowly to the solution while keeping the temperature below 0° C. The solution was stirred at 0° C. for 3 h. The solution was poured into ice water, the solid formed were filtered, washed by cold water, and dried to yield 1.2 g (92%) of white solids. M.P. 122° C. ¹H NMR (DMSO-d₆): 7.60 (dt, J=2.9, 8.5 Hz, 1H), 7.71 (dd, J=2.9, 8.6 Hz, 1H), 8.13 (dd, J=4.8, 8.8 Hz, 1H). EIMS m/z 186 (M+1).

### Preparation of 2-amino-5-fluoro benzoic acid

A solution of 5-fluoro-2-nitrobenzoic acid (10 g, 54 mmol) in ethanol (100 mL) was stirred under hydrogen in the presence of 10% Pd/C (0.5 g) at room temperature for 4 h. The solution was filtered through celite. The solvent was evaporated under reduced pressure to yield 8.2 g (98%) of white solids. M.P. 142° C. ¹H NMR (DMSO-d₆): 6.71 (dd, J=4.9, 8.9 Hz, 1H), 7.15 (dt, J=2.9, 8.4 Hz, 1H), 7.37 (dd, J=2.9, 9.8 Hz, 1H), 8.60 (s, 1H). EIMS m/z 156 (M+1).

### Preparation of 6-fluoro-1H-benzo[d][1,3]oxazine-2,4-dione

Trichloromethyl chloroformate (7.01 mL, 58.13 mmol) was added to a stirred solution of 2-amino-5-fluoro benzoic acid (8.2 g, 52.85 mmol) in dry dioxane at room temperature and the solution was refluxed for 4 h. The solution was cooled in an ice bath and the solids formed were filtered. The solids were washed by ether and dried under vacuum at room temperature to yield 9.1 g (96%) of white solids. M.P. 240° C. ¹H NMR (DMSO-d₆): δ 7.19 (dd, J=4.2, 8.9 Hz, 1H), 7.63-7.71 (m, 1H), 11.77 (s, 1H). EIMS (neg. mode) m/z 180 (M−1). Anal. (C₈H₄FNO₃) C, H, N.

### Preparation of 1-benzyl-6-fluoro-1H-benzo[d][1,3]oxazine-2,4-dione

A solution of 6-fluoro-1H-benzo[d][1,3]oxazine-2,4-dione (3 g, 16.57 mmol) in DMF was added slowly to a suspension of NaH (60% in mineral oil, 729 mg, 18.23 mmol) in DMF and further stirred at room temperature for 1 h. Then, neat benzyl bromide (2.17 mL, 18.23 mmol) was added and the solution was further stirred at room temperature for 3 h. The solution was poured into ice water and the solids formed were filtered, washed several times by water, and dried. The solids were suspended in hexane, sonicated briefly, filtered, and washed by hexane to yield 1.88 g (42%) of white solids. M.P. 95° C. ¹H NMR (DMSO-d₆): δ 5.29 (s, 2H), 7.26 (m, 2H), 7.35 (m, 5H), 7.40 (m, 2H), 7.64 (dt, J=2.9, 8.4 Hz, 1H), 7.82 (dd, J=3.2, 8.0 Hz, 1H). Anal. (C₁₆H₁₃NO₃) C, H, N.

### Preparation of 1-benzyl-6-fluoro-4-hydroxy-2-oxo-1,2-dihydro-quinoline-3-carboxylic acid ethyl ester

Neat diethyl malonate (0.89 mL, 5.8 mmol) was added slowly to a suspension of sodium hydride (60% in mineral oil, 256 mg, 6.41 mmol) in dimethylacetamide under N₂ atmosphere. The mixture was stirred at room temperature until the evolution of hydrogen gas ceased, then the mixture was heated to 90° C. for 30 min and cooled to room temperature. A solution of 1-benzyl-6-fluoro-1H-benzo[d][1,3]oxazine-2,4-dione (1.74 g, 6.41 mmol) in dimethylacetamide was added slowly to the mixture, which was heated overnight at 110° C. The mixture was cooled to room temperature, poured into ice water, and acidified by cold 10% HCl. The solids formed were filtered, washed several times by water, and dried at room temperature under vacuum to yield 1.4 g (64%) of white solids. M.P. 129° C. ¹H NMR (DMSO-d₆): δ 1.30 (t, J=6.9 Hz, 3H), 4.34 (q, J=6.9 Hz, 2H), 5.46 (s, 2H), 7.17-7.24 (m, 5H), 7.38 (dd, J=4.6, 9.6 Hz, 1H), 7.50 (td, J=2.9, 8.3 Hz, 1H), 7.80 (dd, J=3.1, 9.4 Hz, 1H). EIMS m/z 342 (M+1), 364 (M+23). Anal. (C₁₉H₁₆FNO₄) C, H, N.

### Preparation of 1-benzyl-4-chloro-6-fluoro-2-oxo-1,2-dihydro-quinoline-3-carboxylic acid ethyl ester

A solution of 1-benzyl-6-fluoro-4-hydroxy-2-oxo-1,2-dihydro-quinoline-3-carboxylic acid ethyl ester (2.3 g, 6.7 mmol) in 30 mL neat phosphorus oxychloride was heated at 100° C. for 3 h. The solvent was evaporated under reduced pressure. The residue was suspended in ice water and neutralized by solid sodium bicarbonate. The solids formed were filtered, washed by water, and purified by flash chromatography eluting with 1% methanol in dichloromethane to yield 1.6 g (66%) of white solids. Mp. 118° C. ¹H NMR (DMSO-d₆): δ 1.34 (t, J=5.6 Hz, 3H), 4.40 (q, J=5.6 Hz, 2H), 5.55 (s, 2H), 7.19 (d, J=6.0 Hz, 1H), 7.23 (m, 1H), 7.34 (m, 2H), 7.61 (m, 2H), 7.83 (dd, J=2.3, 7.2 Hz, 1H). EIMS m/z 361 (M+1).

### Preparation of 1-benzyl-6-fluoro-4-(4-methyl-piperazin-1-yl)-2-oxo-1,2-dihydro-quinoline-3-carboxylic acid ethyl ester

This compound was prepared from 1-benzyl-4-chloro-6-fluoro-2-oxo-1,2-dihydro-quinoline-3-carboxylic acid ethyl ester by using general procedure A. M.P. 156° C. ¹H NMR (DMSO-d₆): δ 1.32 (t, J=7.2 Hz, 3H), 2.53 (m, 4H), 3.22 (m, 4H), 4.32 (q, J=7.2 Hz, 2H), 5.47 (s, 2H), 7.13 (d, J=11.6 Hz, 1H), 7.24 (m, 1H), 7.32 (m, 2H), 7.46 (m, 2H), 7.56 (dd, J=2.3, 10.0 Hz, 1H). EIMS m/z 424 (M+1).

### Preparation of 1-benzyl-6-fluoro-4-(4-formyl-piperazin-1-yl)-2-oxo-1,2-dihydro-quinoline-3-carboxylic acid ethyl ester

This compound was prepared from 1-benzyl-4-chloro-6-fluoro-2-oxo-1,2-dihydro-quinoline-3-carboxylic acid ethyl ester by using general procedure A. M.P. 206° C. ¹H NMR (DMSO-d₆): δ 1.29 (t, J=7.2 Hz, 3H), 3.10 (m, 4H), 3.70 (m, 4H), 4.32 (q, J=7.2 Hz, 2H), 5.48 (s, 2H), 7.17 (d, J=7.2 Hz, 1H), 7.24 (m, 1H), 7.32 (m, 2H), 7.46 (m, 2H), 7.60 (dd, J=2.3, 6.8 Hz, 1H), 8.09 (s, 1H). EIMS m/z 438 (M+1).

### Preparation of 5-Fluoro-2-nitrobenzoic acid

3-Fluorobenzoic acid (1 g, 7.13 mmol) was dissolved in concentrated H₂SO₄ (2 ml) by warming slightly above room temperature. The solution was cooled to 0° C. Fuming nitric acid (539 mg, 8.56 mmol) was added slowly to the solution while keeping the temperature below 0° C. The solution was stirred at 0° C. for 3 h. The solution was poured into ice water, the solid formed were filtered, washed by cold water, and dried to yield 1.2 g (92%) of white solids. M.P. 122° C. ¹H NMR (DMSO-d₆): 7.60 (dt, J=2.9, 8.5 Hz, 1H), 7.71 (dd, J=2.9, 8.6 Hz, 1H), 8.13 (dd, J=4.8, 8.8 Hz, 1H). EIMS m/z 186 (M+1).

### Preparation of 2-Amino-5-fluoro benzoic acid

A solution of Compound 33 (10 g, 54 mmol) in ethanol (100 mL) was stirred under hydrogen in the presence of 10% Pd/C (0.5 g) at room temperature for 4 h. The solution was filtered through celite. The solvent was evaporated under reduced pressure to yield 8.2 g (98%) of white solids. M.P. 142° C. ¹H NMR (DMSO-d₆): 6.71 (dd, J=4.9, 8.9 Hz, 1H), 7.15 (dt, J=2.9, 8.4 Hz, 1H), 7.37 (dd, J=2.9, 9.8 Hz, 1H), 8.60 (s, 1H). EIMS m/z 156 (M+1).

### Preparation of 6-Fluoro-1H-benzo[d][1,3]oxazine-2,4-dione

Trichloromethyl chloroformate (7.01 mL, 58.13 mmol) was added to a stirred solution of Compound 34 (8.2 g, 52.85 mmol) in dry dioxane at room temperature and the solution was refluxed for 4 h. The solution was cooled in an ice bath and the solids formed were filtered. The solids were washed by ether and dried under vacuum at room temperature to yield 9.1 g (96%) of white solids. M.P. 240° C. ¹H NMR (DMSO-d₆): δ 7.19 (dd, J=4.2, 8.9 Hz, 1H), 7.63-7.71 (m, 1H), 11.77 (s, 1H). EIMS (neg. mode) m/z 180 (M−1). Anal. (C₈ FNO₃) C, H, N.

### Preparation of 6-Fluoro-1-pyridin-2-ylmethyl-1H-benzo[d][1,3]oxazine-2,4-dione

A solution of 6-fluoro-1H-benzo[d][1,3]oxazine-2,4-dione (3.72 g, 20.55 mmol) was added to a suspension of NaH (60% in Min. oil, 1.80 g, 45.21 mmol) in dry DMF (30 mL) stirred at room temperature under argon. The solution was further stirred at room temperature for 30 min. Solid 2-bromomethylpyridine hydrobromide (5.71 g, 22.60 mmol) was added to the solution. The solution was further stirred at room temperature for 3 h and quenched with few drops of water. Excess DMF was evaporated under vacuum. The residue was suspended in water and extracted by CH₂Cl₂. The combined organic phase was dried over MgSO₄ and concentrated to a residue. The crude product was used without further purification.

### Preparation of 4-chloro-6-fluoro-2-oxo-1-pyridin-2-ylmethyl-1,2-dihydro-quinoline-3-carboxylic acid ethyl ester

Neat diethylmalonate (3.37 mL, 22.22 mmol) was added to a suspension of NaH (60% in min. oil, 0.889 g, 22.22 mmol) in dry DMA (60 mL) stirred at room temperature under argon. The solution was stirred at room temperature until the evolution of gas is ceased. Solid 6-fluoro-1-pyridin-2-yl-methyl-1H-benzo[d][1,3]oxazine-2,4-dione (5.5 g, 20.20 mmol) was added to the solution at once. Then the reaction was heated to 110° C. for 5 h. The solution was allowed to come at room temperature and quenched with few drops of water. Excess DMA was evaporated under vacuum and the residue was allowed to stand overnight under high vacuum at room temperature to ensure removal of residual DMA. The residue was dissolved in neat POCl₃ and heated at 90° C. for 3 h. The solution was cooled and excess POCl₃ was evaporated under vacuum. The residue was suspended in saturated NaHCO₃ solution and extracted with CH₂Cl₂. The organic phase was dried over MgSO₄ and concentrated. The crude product was purified by flash chromatography eluting with CH₂Cl₂. Yield 4.2 g (58%). ¹H-NMR (DMSO-d₆): δ 1.32 (t, J=7.2 Hz, 1H), 4.40 (q, J=7.2 Hz, 2H), 5.63 (s, 2H), 7.28 (m, 1H), 7.40 (dd, J=8.0, 1.6 Hz, 1H), 7.60 (m, 2H), 7.80 (m, 2H), 8.44 (m, 1H); EIMS m/z 361 (M+1).

### Preparation of 6-Fluoro-4-(4-methyl-piperazin-1-yl)-2-oxo-1-pyridin-2-ylmethyl-1,2-dihydro-quinoline-3-carboxylic acid ethyl ester

A solution of 4-chloro-6-fluoro-2-oxo-1-pyridin-2-ylmethyl-1,2-dihydro-quinoline-3-carboxylic acid ethyl ester (541 mg, 1.5 mmol) and 1-methyl piperazine (0.183 mL, 16.5 mmol) and Dabco (336 mg, 3 mmol) in DMA (10 mL) was heated at 110° C. for 2 h. The solution was cooled and the solvent was evaporated under vacuum. The residue was suspended in water, sonicated briefly and filtered. The crude product was purified by flash chromatography eluting with 0-5% MeOH in CH₂Cl₂ gradient within 100 min. with a flow rate of 15 mL/min through RediSep® SiO₂ column (4 g) in combiflash system (Isco). ¹H-NMR (DMSO-d₆): δ 1.29 (t, J=6.8 Hz, 1H), 2.26 (s, 3H), 2.66 (m, 4H), 3.10 (m, 4H), 4.28 (q, J=6.8 Hz, 2H), 5.54 (s, 2H), 7.22 (m, 2H), 7.44 (m, 2H), 7.56 (d, J=8.8 Hz, 1H), 7.76 (m, 1H), 8.47 (d, J=4.4, Hz, 1H); EIMS m/z 425 (M+1).

### Preparation of 5-methyl-1-phenyl-1H-indole-2,3-dione

A solution of 5-methylisatin (25.3 g, 0.157 mol), iodobenzene (26.3 mL, 0.236 mol), and CuO (25.0 g, 0.314 mol) in DMF (190 mL) was heated at 170° C. for 17 hours. The reaction was cooled to room temperature and the insoluble solids were filtered out and washed well with cold DMF. The filtrate was poured into 2.5 liters ice water containing 125 g NaCl. The precipitated solid product was filtered out, dried and purified by flash chromatography eluting with isocratic dichlormethane. The combined fractions containing the product was pooled and concentrated to a residue. The residue was then dissolved in a small amount of CH₂Cl₂, and diluted by adding excess isopropyl ether. This precipitated formed was collected by filtration. Yield 9.09 g (24%). ¹H-NMR (DMSO-d₆): δ 2.30 (s, 3H), 6.74 (d, J=8.4 Hz, 1H), 7.47 (m, 5H), 7.58 (m, 2H); EIMS m/z 254 (M+1).

### Preparation of 6-methyl-1-phenyl-1H-benzo[d][1,3]oxazine-2,4-dione

Meta-Chloroperbenzoic acid (mCPBA, 8.59 g, 38.3 mmol) was added slowly to a solution of 5-methyl-1-phenyl-1H-indole-2,3-dione (8.5 g, 35.8 mmol) in anhydrous dichloromethane. The reaction was exothermic enough to make the CH₂Cl₂ boil for 5 minutes. After stirring at r.t. for 1 hour, the insoluble solids were filtered out and discarded. The filtrate was diluted with CH₂Cl₂ and washed water containing 3 equiv potassium carbonate (14.9 g), then dried over magnesium sulfate. The CH₂Cl₂ was removed under reduced pressure to give 8.82 g crude impure product. This impure product was used without further purification.

### Preparation of 4-hydroxy-6-methyl-2-oxo-1-phenyl-1,2-dihydro-quinoline-3-carboxylic acid ethyl ester

Diethylmalonate (3.67 mL, 24.2 mmol) was added slowly over a 5 minute period to a suspension of NaH (1.23 g, 30.8 mmol) anhydrous DMF (60 mL) under argon at 0° C. Solid 6-methyl-1-phenyl-1H-benzo[d][1,3]oxazine-2,4-dione (6.0 g, 23.7 mmol) was then added, and the reaction was heated to 110° C. for 1 hour. The reaction mixture was cooled to r.t., then poured into 600 mL water containing 2 equiv of potassium carbonate (6.55 g). The solution was then washed twice with 250 mL ethyl acetate. The aqueous phase was then acidified slowly with 4M HCl to pH 2. The precipitated product was then filtered out and allowed to dry in the air overnight. Yield 4.05 grams (53%).

¹H-NMR (DMSO-d₆): δ 1.27 (t, J=7.2 Hz, 3H), 2.36 (s, 3H), 4.31 (q, J=7.2 Hz, 2H), 6.38 (d, J=8.8 Hz, 1H), 7.29 (m, 2H), 7.37 (dd, J=1.6, 8.4 Hz, 1H), 7.55 (m, 1H), 7.60 (m, 2H), 7.88 (s, 1H), 13.36 (b, 1H); EIMS m/z 324 (M+1)

### Preparation of 4-chloro-6-methyl-2-oxo-1-phenyl-1,2-dihydro-quinoline-3-carboxylic acid ethyl ester

Oxalyl chloride (3.24 mL, 37.1 mmol) was added very slowly and carefully to anhydrous DMF (60 mL) stirred at −50° C. under argon. To this solution was added solid ethyl 1,2-dihydro-4-hydroxy-6-methyl-2-oxo-1-phenylquinoline-3-carboxylate (4.0 g, 12.4 mmol). The reaction mixture was heated at 75° C. for 3 hours. The reaction was then cooled to r.t. and poured into 600 mL ice water containing 120 g NaCl. The precipitated product was then filtered out, dissolved in CH₂Cl₂ and then dried over magnesium sulfate. Evaporation of the CH₂Cl₂ gave 4.32 g of the product (97%) yield.

¹H-NMR (DMSO-d₆): δ 1.30 (t, J=7.2 Hz, 3H), 2.41 (s, 3H), 4.35 (q, J=7.2 Hz, 2H), 6.52 (d, J=8.4 Hz, 1H), 7.40 (m, 2H), 7.44 (dd, J=2.0, 8.8 Hz, 1H), 7.62 (m, 3H), 7.86 (s, 1H); EIMS m/z 342 (M+1)

### Preparation of 6-methyl-2-oxo-1-phenyl-4-(piperazin-1-yl)-1,2-dihydro-quinoline-3-carboxylic acid ethyl ester

This compound was prepared from 4-chloro-6-methyl-2-oxo-1-phenyl-1,2-dihydro-quinoline-3-carboxylic acid ethyl ester (0.7 g, 2.05 mmol) and piperazine (0.882 g, 10.2 mmol) by using general procedure B. Yield 0.54 g. (67%). ¹H-NMR (DMSO-d₆): δ 1.32 (t, J=7.2 Hz, 3H), 2.40 (s, 3H), 2.97 (m, 4H), 3.11 (m, 4H), 4.29 (q, J=7.2 Hz, 2H), 6.46 (d, J=8.4 Hz, 1H), 7.32 (m, 3H), 7.55-7.65 (m, 3H), 7.73 (s, 1H); EIMS m/z 392 (M+1); M.P. 181° C.

### Preparation of 6-methyl-4-(4-methylpiperazin-1-yl)-2-oxo-1-phenyl-1,2-dihydro-quinoline-3-carboxylic acid ethyl ester

This compound was prepared from 4-chloro-6-methyl-2-oxo-1-phenyl-1,2-dihydro-quinoline-3-carboxylic acid ethyl ester and N-methylpiperazine (0.62 g, 6.14 mmol) by using general procedure B. Yield 0.40 g. (48%). ¹H-NMR (DMSO-d₆): δ 1.28 (t, J=7.2 Hz, 3H), 2.29 (s, 3H), 2.37 (s, 3H), 2.57 (b, 4H), 3.15 (b, 4H), 4.27 (q, J=7.2 Hz, 2H), 6.43 (d, J=8.4 Hz, 1H), 7.30 (m, 3H), 7.50-7.65 (m, 3H), 7.67 (s, 1H); EIMS m/z 406 (M+1); M.P. 188° C.

### Preparation of 4-(4-formylpiperazin-1-yl)-6-methyl-2-oxo-1-phenyl-1,2-dihydro quinoline-3-carboxylic acid ethyl ester

4-chloro-6-methyl-2-oxo-1-phenyl-1,2-dihydro-quinoline-3-carboxylic acid ethyl ester (0.7 g, 2.05 mmol) and N-formylpiperazine (0.70 g, 6.14 mmol) were dissolved in 5 mL CH₂Cl₂ and heated at 40° C. for 12 hours. The CH₂Cl₂ was washed 3 times with water containing 3 equiv sodium bisulfate (0.74 g). The organic phase was dried over MgSO₄ and concentrated. The crude product was purified by flash chromatography eluting with 0-20% MeOH in CH₂Cl₂ gradient within 200 min. Yield 0.12 g (14%). ¹H-NMR (DMSO-d₆): δ 1.27 (t, J=7.2 Hz, 3H), 2.39 (s, 3H), 3.14 (m, 4H), 3.64 (m, 4H), 4.27 (q, J=7.2 Hz, 2H), 6.45 (d, J=8.4 Hz, 1H), 7.30 (m, 3H), 7.5-7.6 (m, 3H), 7.76 (s, 1H), 8.12 (s, 1H); EIMS m/z 420 (M+1); M.P. 240° C.

### Preparation of 1-Phenyl-1H-benzo[d][1,3]oxazine-2,4-dione

Trichloromethyl chloroformate (13.30 mL, 110.27 mmol) was added slowly to a solution of N-phenylanthranilic acid (17 g, 79.51 mmol) in anhydrous dioxane (500 mL) stirred at room temperature under argon. While adding trichloromethyl chloroformate, the temperature of solution was maintained to room temperature. After completion of addition, the solution was heated at 110° C. for 5 h. The solution was cooled to room temperature and excess solvent was evaporated. The residue was suspended in diethyl ether, sonicated briefly and filtered. Yield 18.20 g (96%). M.P. 183° C. ¹H NMR (DMSO-d₆): δ 6.42 (d, J=8.4 Hz, 1H), 7.34 (m, 1H), 7.50 (m, 2H), 7.58 (m, 1H), 7.64 (m, 3H), 8.07 (dd, J=7.6. 1.6, 1H). EIMS m/z 240 (M+1).

### Preparation of 4-hydro-2-oxo-1-phenyl-1,2-dihydro-quinoline-3-carboxylic acid ethyl ester

Diethylmalonate (5.71 mL, 37.62 mmol) was added slowly to a suspension of NaH (1.65 g, 41.38 mmol) anhydrous DMF (60 mL) stirred at room temperature under argon. Solid 1-phenyl-1H-benzo[d][1,3]oxazine-2,4-dione (9.0 g, 37.62 mmol) was then added, and the reaction was heated overnight to 110° C. The reaction mixture was cooled to r.t. and excess solvent was evaporated under vacuum. The residue was suspended in water (most of them was dissolved) and acidified to pH 2-3 with cold 10% HCl. The solids formed were filtered, washed with cold water and air dried. Yield 10.20 g (87%). ¹H-NMR (DMSO-d₆): δ 1.28 (t, J=7.2 Hz, 3H), 4.31 (q, J=7.2 Hz, 2H), 6.48 (d, J=8.8 Hz, 1H), 7.30 (m, 3H), 7.51 (m, 2H), 7.61 (m, 2H), 8.10 (dd, J=8.0, 1.6 Hz, 1H); EIMS m/z 310 (M+1).

### Preparation of 4-chloro-2-oxo-1-phenyl-1,2-dihydro-quinoline-3-carboxylic acid ethyl ester

A solution of 4-hydroxy-2-oxo-1-phenyl-1,2-dihydro-quinoline-3-carboxylic acid ethyl ester (10.20 g, 32.97 mmol) in neat POCl₃ was heated at 90° C. for 3 h. The solution was then cooled and excess POCl₃ was distilled off under vacuum. The residue was suspended in ice water, sonicated briefly and filtered. The solids were dissolved in CH₂Cl₂, washed successively by saturated NaHCO₃ solution, water and brine. The organic phase was dried over MgSO₄ and concentrated to a residue. Yield 10.02 g (93%). ¹H-NMR (DMSO-d₆): δ ¹H-NMR (DMSO-d₆): δ 1.30 (t, J=7.2 Hz, 3H), 4.37 (q, J=7.2 Hz, 2H), 6.63 (d, J=8.4 Hz, 1H), 7.42 (m, 3H), 7.60 (m, 4H), 8.08 (dd, J=8.0, 1.6 Hz, 1H); EIMS m/z 328 (M+1).

### Preparation of 2-oxo-1-phenyl-4-(piperazin-1-yl)-1,2-dihydro-quinoline-3-carboxylic acid ethyl ester

This compound was prepared from 4-chloro-2-oxo-1-phenyl-1,2-dihydro-quinoline-3-carboxylic acid ethyl ester (10 g, 30.51 mmol) and piperazine (7.88 g, 91.52 mmol) by using general procedure B. Yield 8.40 g. (73%). ¹H-NMR (DMSO-d₆): δ ¹H-NMR (DMSO-d₆): δ 1.30 (t, J=7.2 Hz, 3H), 2.93 (m, 4H), 3.09 (m, 4H), 4.28 (q, J=7.2 Hz, 2H), 6.51 (d, J=8.4 Hz, 1H), 7.31 (m, 3H), 7.45 (m, 1H), 7.55 (m, 1H), 7.62 (m, 2H), 8.14 (d, J=8.0 Hz, 1H); EIMS m/z 378 (M+1).

### Preparation of 4-(4-methyl-piperazin-1-yl)-2-oxo-1-phenyl-1,2-dihydro-quinoline-3-carboxylic acid ethyl ester

Solid 2-oxo-1-phenyl-4-(piperazin-1-yl)-1,2-dihydro-quinoline-3-carboxylic acid ethyl ester (755 mg, 2 mmol) was added slowly to a suspension of NaH (60% in Min. Oil, 72 mg, 1.8 mmol) in DMF stirred at room temperature under argon. The solution was further stirred at room temperature for 30 min. Neat CH₃I (256 mg, 1.8 mmol) was added slowly via syringe to the solution and further stirred for 2 h. The reaction was quenched by adding few drops of water. The solvent was evaporated under vacuum. The residue was suspended in water and basified by saturated NaHCO₃ solution. The solids formed were filtered, washed by cold water and dried at room temperature. The crude product was purified by flash chromatography eluting with 0-5% MeOH in CH₂Cl₂ gradient within 100 min with a flow rate of 20 mL/mm in SiO₂ (12 g) column in a combiflash. Yield 470 mg (67%). ¹H-NMR (DMSO-d₆): δ ¹H-NMR (DMSO-d₆): δ 1.29 (t, J=7.2 Hz, 3H), 2.30 (s, 1H), 2.55 (m, 4H), 3.15 (m, 4H), 4.28 (q, J=7.2 Hz, 2H), 6.53 (d, J=8.4 Hz, 1H), 7.31 (m, 3H), 7.46 (m, 1H), 7.52 (m, 1H), 7.59 (m, 2H), 8.14 (d, J=8.0 Hz, 1H); EIMS m/z 392 (M+1).

### TNF-Alpha as a Drug Target

TNF-alpha can be well suited for analysis as a drug target as its activity has been implicated in a variety of pathophysiological conditions. TNF-alpha inhibitors of preferred embodiments inhibit lethality in mice following LPS challenge. Accordingly, a variety of inflammatory conditions can be amenable to treatment with a TNF-alpha inhibitor. In this regard, among other advantages, the inhibition of TNF-alpha activity and/or release can be employed to treat inflammatory response and shock. Beneficial effects can be achieved by intervention at the early stage of the shock response.

TNF-alpha inhibitors can be useful in a variety of TNF-alpha associated disease states including transplant rejection, immune-mediated and inflammatory elements of CNS disease (e.g., Alzheimer's, Parkinson's, multiple sclerosis, etc.), muscular dystrophy, diseases of hemostasis (e.g., coagulopathy, veno occlusive diseases, etc.), allergic neuritis, granuloma, diabetes, graft versus host disease, chronic renal damage, alopecia (hair loss), acute pancreatitis, joint disease, congestive heart failure, cardiovascular disease (restenosis, atherosclerosis), joint disease, and osteoarthritis.

TNF-alpha inhibitors of the preferred embodiments include compounds having the following structures as provided in Table 1.

TABLE 1

| | | | Molecular |
|---|---|---|---|
| Compound No. | Structure | Formula | Wt. |
| 100 | 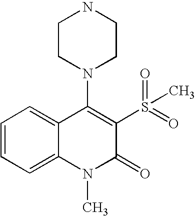 | C15 H19 N3 O3 S | 321.3991 |
| 101 | 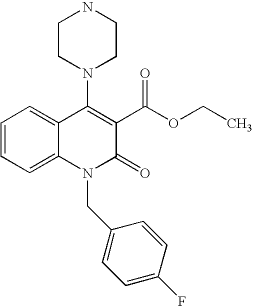 | C23 H24 F N3 O3 | 409.4586 |
| 102 | 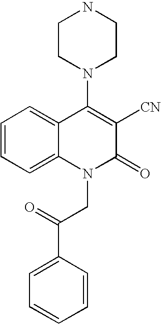 | C22 H20 N4 O2 | 372.426 |
| 103 | 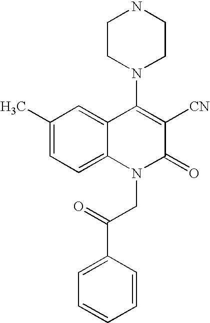 | C23 H22 N4 O2 | 386.4528 |
| 104 | 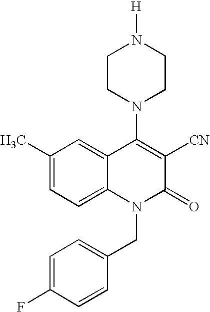 | C22 H21 F N4 O | 376.4329 |
| 105 | 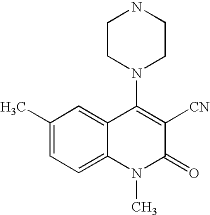 | C16 H18 N4 O | 282.3452 |
| 106 | 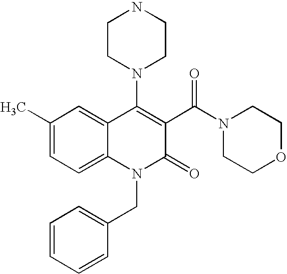 | C26 H30 N4 O3 | 446.548 |
| 107 | 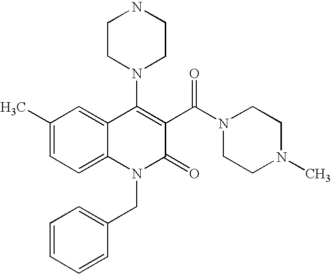 | C27 H33 N5 O2 | 459.5907 |
| 108 | 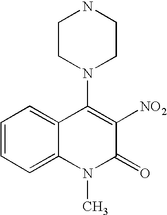 | C14 H16 N4 O3 | 288.3054 |
| 109 | 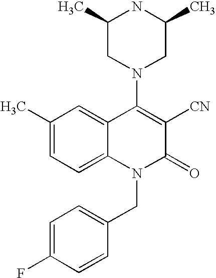 | C24 H25 F N4 O | 404.4865 |
| 110 | 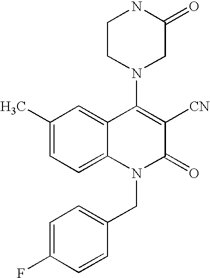 | C22 H19 F N4 O2 | 390.4161 |
| 111 | 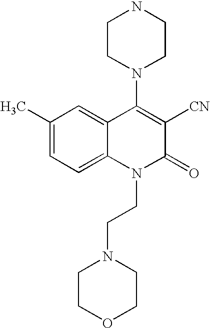 | C21 H27 N5 O2 | 381.4773 |
| 112 | 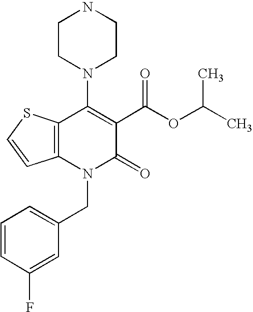 | C22 H24 F N3 O3 S | 429.5136 |
| 113 | 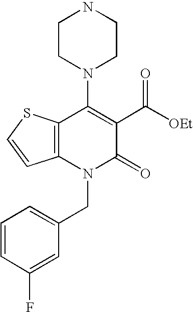 | C21 H22 F N3 O3 S | 415.4868 |
| 114 | 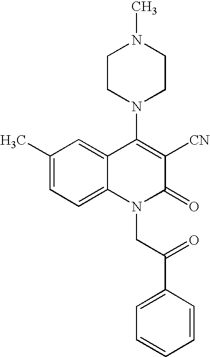 | C24 H24 N4 O2 | 400.4796 |
| 115 | 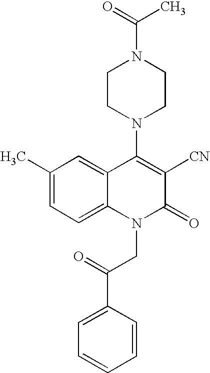 | C25 H24 N4 O3 | 428.4896 |
| 116 | 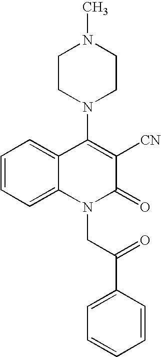 | C23 H22 N4 O2 | 386.4528 |
| 117 | 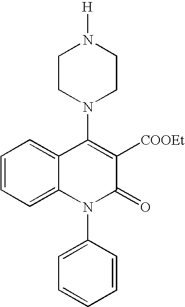 | C22 H23 N3 O3 | 377.4417 |
| 118 | 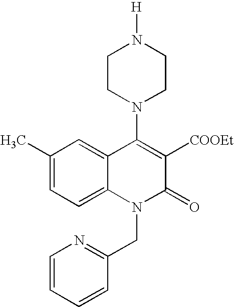 | C23 H26 N4 O3 | 406.4834 |
| 119 | 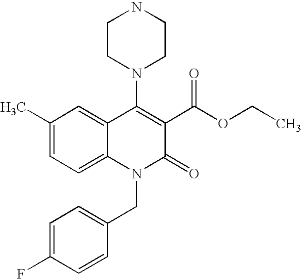 | C24 H26 F N3 O3 | 423.4854 |
| 120 | 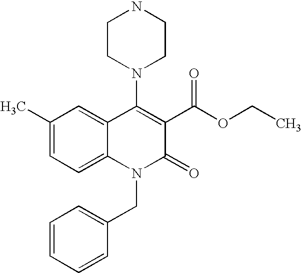 | C24 H27 N3 O3 | 405.4953 |
| 121 | 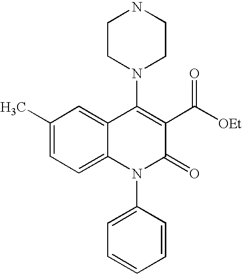 | C23 H25 N3 O3 | 391.4685 |
| 122 | 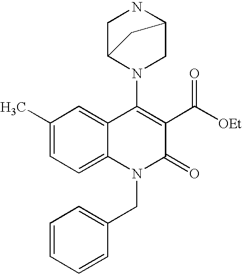 | C25 H27 N3 O3 | 417.5063 |
| 123 | 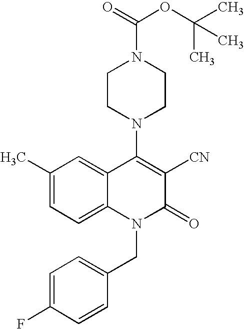 | C27 H29 F N4 O3 | 476.5491 |
| 124 | 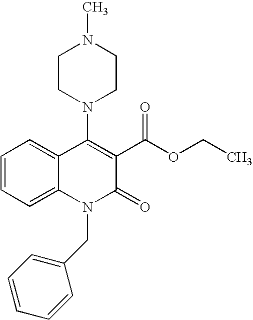 | C24 H27 N3 O3 | 405.4953 |
| 125 | 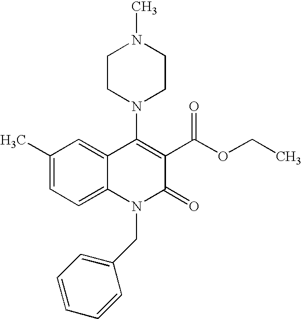 | C25 H29 N3 O3 | 419.5221 |
| 126 | 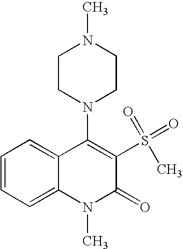 | C16 H21 N3 O3 S | 335.4259 |
| 127 | 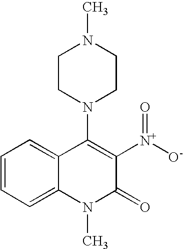 | C15 H18 N4 O3 | 302.3322 |
| 128 | 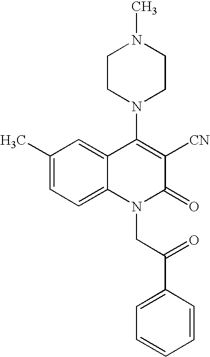 | C24 H24 N4 O2 | 400.4796 |
| 129 | 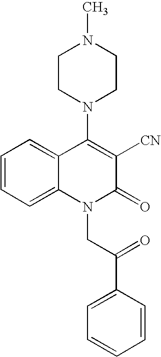 | C23 H22 N4 O2 | 386.4528 |
| 130 | 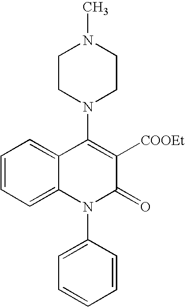 | C23 H25 N3 O3 | 391.4685 |
| 131 | 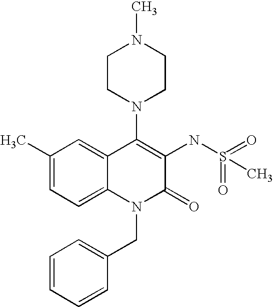 | C23 H28 N4 O3 S | 440.5652 |
| 132 | 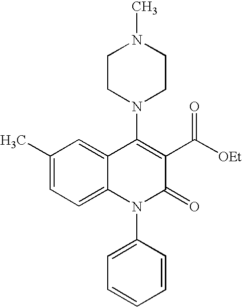 | C24 H27 N3 O3 | 405.4953 |
| 133 | 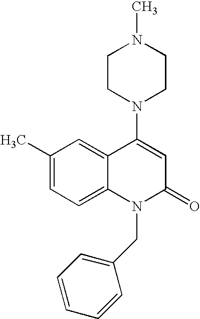 | C22 H25 N3 O | 347.4595 |
| 134 | 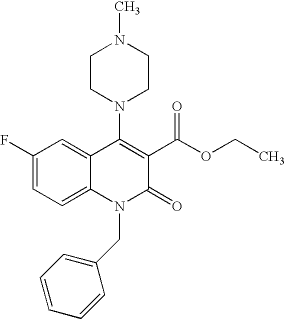 | C24 H26 F N3 O3 | 423.4854 |
| 135 | 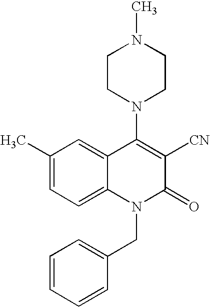 | C23 H24 N4 O | 372.4696 |
| 136 | 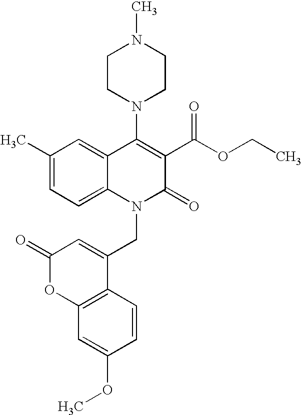 | C29 H31 N3 O6 | 517.5789 |
| 137 | 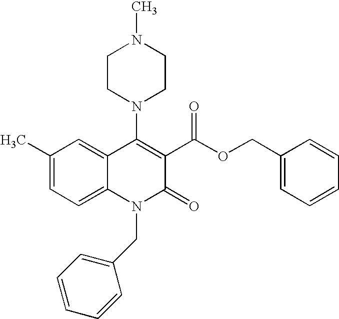 | C30 H31 N3 O3 | 481.5929 |
| 138 | 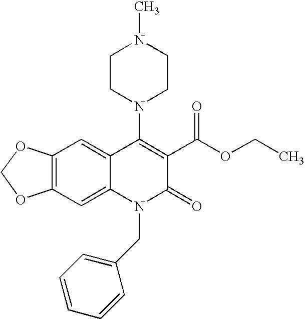 | C25 H27 N3 O5 | 449.5043 |
| 139 | 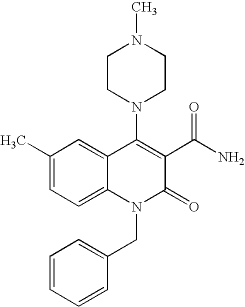 | C23 H26 N4 O2 | 390.4844 |
| 140 | 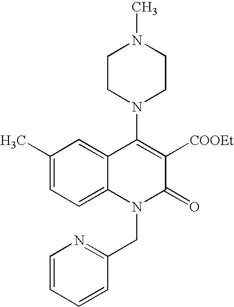 | C24 H28 N4 O3 | 420.5102 |
| 141 | 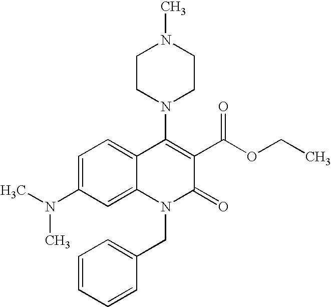 | C26 H32 N4 O3 | 448.5638 |
| 142 | 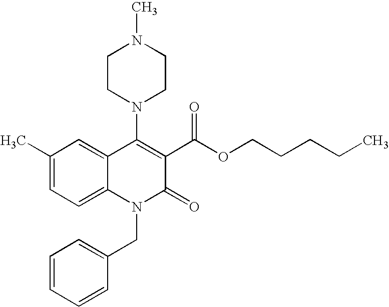 | C28 H35 N3 O3 | 461.6025 |
| 143 | 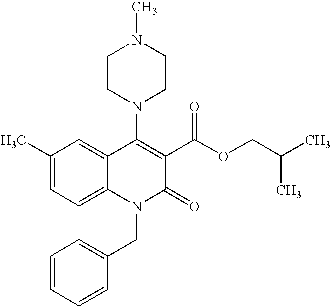 | C27 H33 N3 O3 | 447.5757 |
| 144 | 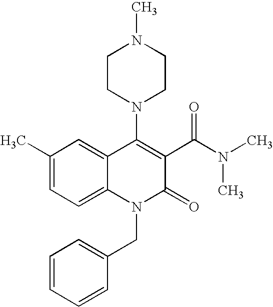 | C25 H30 N4 O2 | 418.538 |
| 145 | 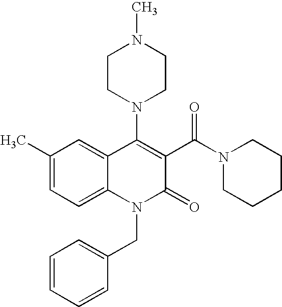 | C28 H34 N4 O2 | 458.6026 |
| 146 | 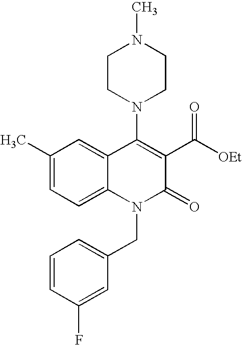 | C25 H28 F N3 O3 | 437.5122 |
| 147 | 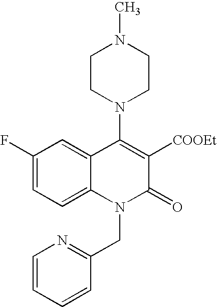 | C23 H25 F N4 O3 | 424.4735 |
| 148 | 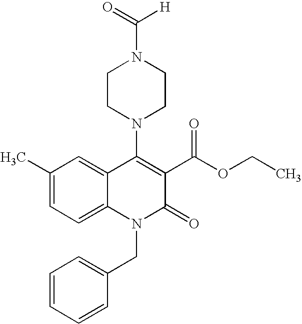 | C25 H27 N3 O4 | 433.5053 |
| 149 | 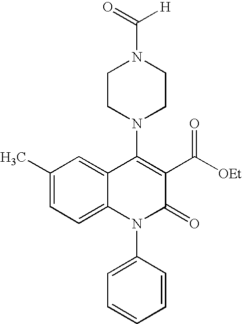 | C24 H25 N3 O4 | 419.4785 |
| 150 | 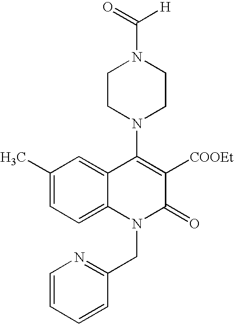 | C24 H26 N4 O4 | 434.4934 |
| 151 | 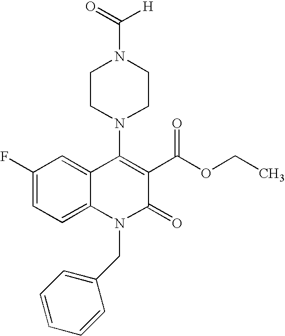 | C24 H24 F N3 O4 | 437.4686 |
| 152 | 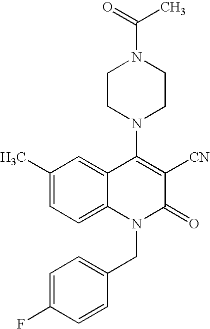 | C24 H23 F N4 O2 | 418.4697 |
| 153 | 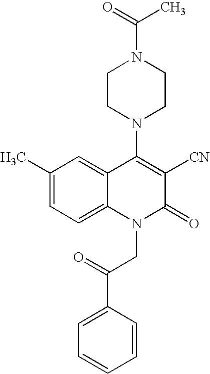 | C25 H24 N4 O3 | 428.4896 |
| 154 | 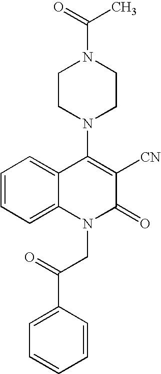 | C24 H22 N4 O3 | 414.4628 |
| 155 | 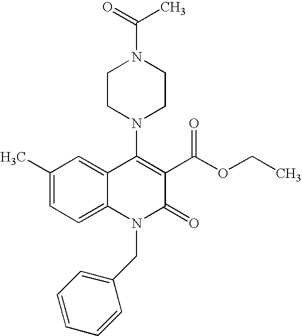 | C26 H29 N3 O4 | 447.5321 |
| 156 | 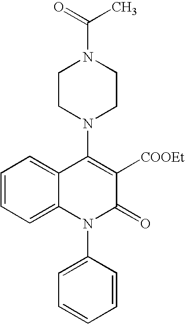 | C24 H25 N3 O4 | 419.4785 |

The activity of selected TNF-alpha inhibitors in Table 1 was demonstrated in in vivo testing. Mice were challenged with LPS to produce TNF-alpha in vivo, and then assayed by ELISA after dosing with or without the TNF-alpha inhibitor (formulated in FS-1 and delivered through oral gavage). Each of the TNF-alpha inhibitors exhibited inhibition of TNF-alpha production, as shown by the data in Table 2.

TABLE 2

| Compound | % Inhibition @ | | |
|---|---|---|---|
| Number | 10 mpk; P.O. | SD | N |
| 101 | 43.4 | | 1 |
| 102 | 36.5 | 4.2 | 4 |
| 103 | 67.3 | 4.1 | 2 |
| 113* | 84.1 | 6.6 | 2 |
| 114 | 51.6 | | 1 |
| 115 | 41.3 | | 1 |
| 116 | 29.4 | | 1 |
| 105 | 21.0 | | |
| 108 | 26.2 | 0.5 | 2 |

*tested at 3 mg/kg

### EXPERIMENT

NVP-VAR235-NX was identified as a main metabolite of NVP-VAQ996-NX after in vitro incubation with liver microsomes of mouse and human and in vivo in plasma after per os (p.o.) administration to mice.

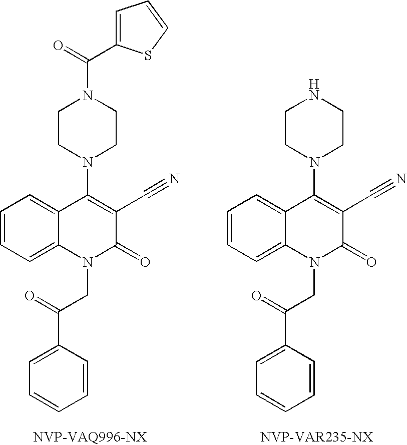

This metabolite is probably formed by cleavage of the thiophene-2-carboxamide bond and was also detected after incubation of NVP-VAQ996-NX in freshly prepared mouse plasma.

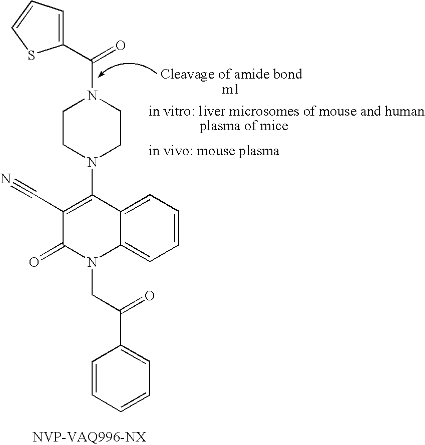

In Vitro Incubation with Liver Microsomes of Mouse and Human

Stock solutions of NVP-VAQ996-NX-1 (2 mmol/L) were prepared in DMSO. Alamethicin solution was prepared (0.125 mmol/L) in water. Uridine 5′-diphosphoglucuronic acid (UDPGA) solution (24 mmol/L) was prepared in phosphate buffer 100 mM pH 7.4. In a further experiment 50 mmol/L gluthationic acid reduced (GSH) in phosphate buffer 100 mM pH 7.4 was added as second co-factor.

For the characterization of in vitro metabolites NVP-VAQ996-NX-1 was incubated at 37° C. for up to 60 minutes with liver microsomes from mouse and human (Table 3).

TABLE 3
Type and concentration of liver microsomes

| | | Protein | P450 protein |
|---|---|---|---|
| Species | Batch | concentration | concentration |
| Mouse (M) | Gentest 452701 | 20 mg/ml | 810 pmol/mg |
| | Charge 11 | | |
| Rat (R) | Gentest 452161 | 20 mg/ml | 320 pmol/mg |
| | Charge 22 | | |

3 μL liver microsomes, containing 20 mg protein/mL, were mixed with 417 μL phosphate buffer, 60 μL alamethicin solution and 60 μL UDPGA. To this reaction mixture 3 μL stock solution (2 mM in DMSO) of NVP-VAQ996-NX-1 was added and pre-incubated for 3 min at 37° C. After pre-incubation, the final reaction was started by addition of 60 μL of the nicotinamide-adenine-dinucleotide-phosphate (NADPH)-regenerating system, containing isocitrate-dehydrogenase (TU/mL), NADP (1 mmol/L), isocitrate (5 mmol/L). After 1 h, the reaction was stopped with 600 μL ice-cold acetonitrile. Experiments were conducted according to a generic protocol. The reaction mixture was stored at −80° C. The final set-up of the in vitro incubation is shown on Table 4.

TABLE 4
Final set-up of in vitro incubations for NVP-VAQ996-NX with liver micrcosomes of
mouse and human

| | Samples | | | | | |
|---|---|---|---|---|---|---|
| | 0 min | 60 min | 60 min | 0 min | 60 min | |
| | (XY1)** | (XY2) | (XY3) | (XY4) | (XY5) | Final |
| Ingredient | [μl] | [μl] | [μl] | [μl] | [μl] | conc. |
| Microsomes** dil. 2 mg protein/ml | 25 | 25 | 25 | 25 | 25 | 0.1 mg/ml |
| Phosphate buffer 100 mM, pH 7.4 | 372.5 | 322.5 | 372.5 | 322.5 | 222.5 | |
| Alamethicin Sol. (0.5 mM | 1.25 | 1.25 | 1.25 | 1.25 | 1.25 | 1.25 μM |
| in EtOH) | | | | | | |
| UDPGA-Sol. (24 mM in H₂O) | 50 | 50 | 50 | 50 | 50 | 2.4 mM |
| GSH-Sol. (15 mM in Puffer) | | | | 50 | | 1.5 mM |
| GSH-Sol. (100 mM in Puffer) | | | | | 50 | 10 mM |
| VAQ996-Sol. 2 mM in DMSO | 1.25 | 1.25 | 1.25 | 1.25 | 1.25 | 5 μM |
| NADPH-Reg-System | 50 | 50 | 50 | 50 | 50 | |

*Species: rat, mouse, dog, minipig, cynomolgus monkey, human
**XY: Mouse = M, Human = H3

Sample Preparation of In Vitro Samples

Prior to use, the mixture was centrifuged (10000 g, 5 min) and 500 μL of 1 mL supernatant evaporated to dryness. The residue was diluted in 200 μl (in vitro) and 500 μl (in vivo), respectively, acetonitril/water (2/8). From this final solution aliquots of 10 μL were used for analysis.

In Vitro Incubation in Plasma from Mice

Heparinized mouse plasma was freshly obtained from Balb/c mice. Samples were incubated with 5 μg/ml NVP-VAQ996 added from a stock solution (1 mg/ml) in DMSO for 2 or 24 hrs at 37° C.

Administration to Mice and Sampling

Female Balb/c mice (Charles River) were housed under standard conditions with free access to food and drinking water throughout the experiment. The mice (n=4) were treated perorally with 10 mg/kg NVP-VAQ996-NX-1 (at 5 mL/kg) by gavage. At the allotted time points mice blood samples were obtained into heparinized tubes (30 IU/mL). The liver was removed intact, weighed and snap frozen before storage at −70° C. until analyzed.

Preparation of Plasma Samples

For protein precipitation, to 100 μL plasma 100 μL ice-cold acetonitrile was added. This procedure was repeated twice during 1 hour and, after centrifugation (10000 g, 5 min), the supernatant was used for further preparation.

Final Preparation of Plasma Samples

The solvent of the supernatant was evaporated using the Cyclone high speed evaporator (Prolab, Reinach, Switzerland) and the residue reconstituted with water/acetonitrile (95/5; v/v). An aliquot of the final solution was used for the high performance liquid chromatography/mass spectrometry (HPLC/MS) analyses.

Capillary-HPLC/MS⁽ⁿ⁾ Analysis

The liquid chromatographic separation was performed using an Chorus-220 syringe pump (CS analytics, Beckenried, Switzerland) and a home-made glass capillary column, 150 mm×0.3 mm, filled with Nucleosil C18-HD, particle size 3.5 μm Gradient mobile phase programming was used with a flow rate of 4.5 μL/min. Eluent A was acetonitrile/water (5/95) 10 mM HCOONH₄+0.02% trifluoroacetic acid (TFA). Eluent B was acetonitrile/methanol/water (9/5/5)+10 mM HCOONH₄+0.02% TFA. The mobile phase was held 2 min. isocratic at 5% B, followed by a linear gradient from 15% B to 95% B over 30 min and a 5 min isocratic phase at 95% B. The column temperature was kept at 40° C.

The column effluent was introduced directly into the ion source of a linear ion trap (LTQ, Thermo, San Jose, Calif.) or a triple stage quadrupole mass spectrometer (TSQ Quantum, Thermo Sam Jose, Calif.). The ionization technique employed was positive electrospray (+ES). Both mass spectrometers were used either in the full scan mode (m/z 250-m/z 1000) or in product ion scan mode. The collision energy (CE) was set at 25% (normalized CE, LTQ) or at 22-28V in case of the Quantum (collision gas: 1.5 mT argon)

Results

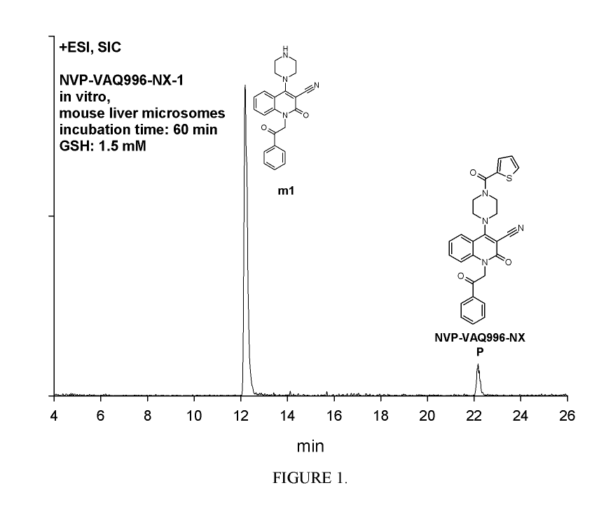

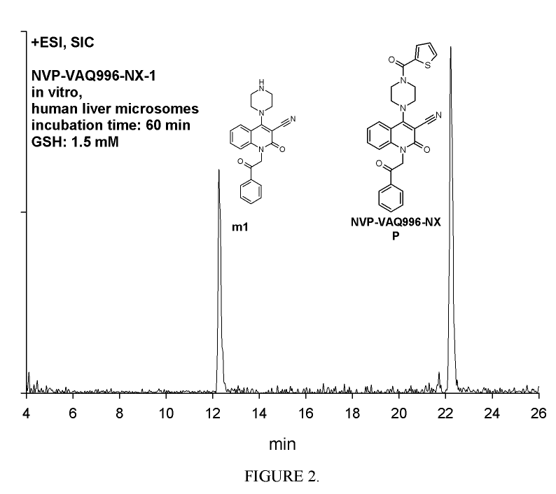

Metabolite identification of NVP-VAQ996-NX was performed using the in vitro samples obtained by incubation in mouse and human liver microsomes. The metabolic degradation of NVP-VAQ996 was high with liver microsomes of mouse and human. In addition, one metabolite (ml) could be characterized as shown on a representative ion chromatogram (FIG. 1). The uncorrected peak area of NVP-VAQ996-NX and metabolites across different species is shown on FIG. 2.

Incubation of NVP-VAQ996-NX in plasma from mice shows also high rate of degradation to metabolite ml.

In vivo, three additional metabolites (m3, m4 and m6) were characterized in plasma and liver 2 hours after p.o. administration of 10 mg/kg NVP-VAQ996-NX to mice. Metabolites m3 and m4 are glucuronides of hydroxymetabolites. In case of metabolite m6, no structure proposal was possible, but some characteristic mass spectral data are available (see Table 5).

TABLE 5
Mass spectral data of NVP-VAQ996-NX (P), proposed in
vitro and in vivo metabolite (ml) and NVP-VAR235-NX

| Parent/ | MH+ | H/D | Fragments |
|---|---|---|---|
| Metabolite No | [m/z] | # | [m/z] |
| VAQ996 | 483 | 0 | 356, 371, 398, 438 |
| m1 | 373 | 1 | 250, 303, 329, 356 |
| VAR235 | 373 | 1 | 250, 303, 329, 356 |

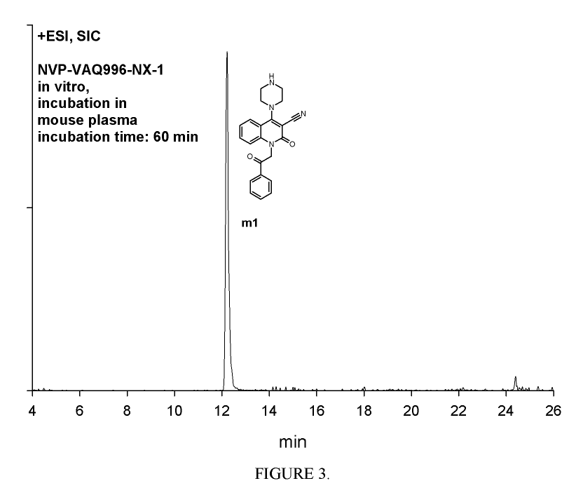

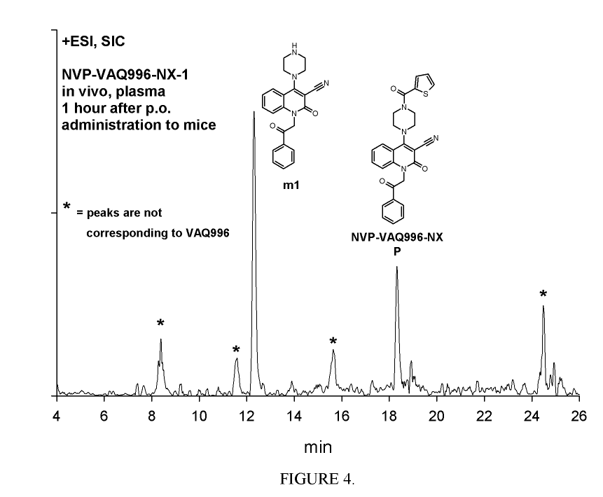

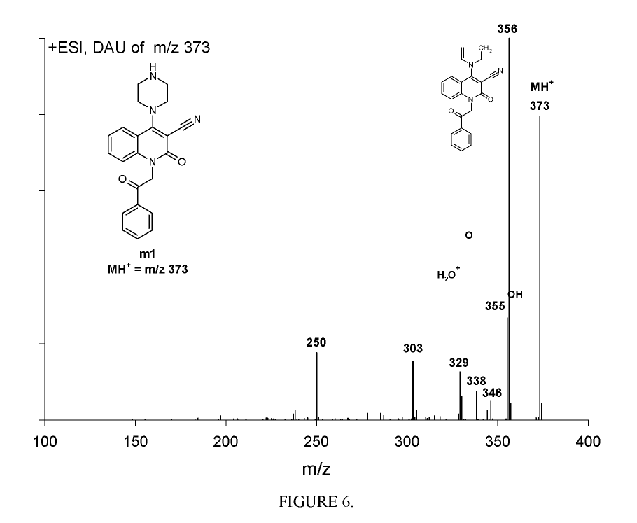

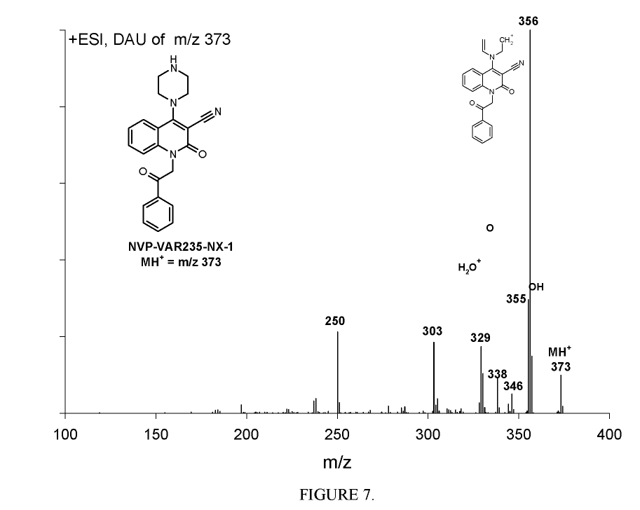

Representative ion chromatograms of plasma, liver and muscle are shown in FIGS. 3 to 5. The relative proportions of unchanged parent compound and metabolites were determined semi-quantitatively without consideration of the respective response factors as shown on FIGS. 6 to 7.

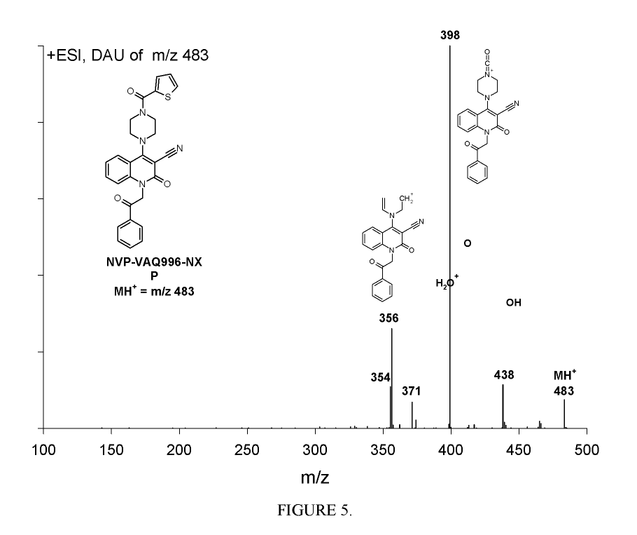

NVP-VAQ996-NX and metabolite ml were characterized by their electro spray ionization (ESI) product ion spectra on quasi-molecular ions (MH⁺) as shown on FIGS. 5 and 6. The number exchangeable hydrogen atoms of the proposed metabolite were confirmed by the H/D exchange experiment. The summarized mass spectral data of NVP-VAQ996-NX (P) and proposed metabolite ml in shown in Table 5.

The proposed metabolite ml was corroborated by the reference compound NVP-VAR235-NX.

NVP-VAR235-NX was identified as main metabolite of NVP-VAQ996-NX after in vitro incubation with liver microsomes of mouse and human and in vivo in plasma after p.o. administration to mice. This metabolite was also formed after incubation of NVP-VAQ996-NX in freshly prepared mice plasma. The main metabolic pathway is as follows. Due to the formation of ml after incubation of VAQ996 in native mouse plasma the formation of ml is probably not only P450 mediated but also formed by amidases.

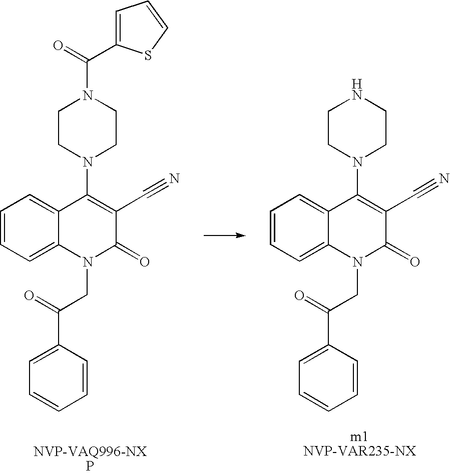

Other phase I and/or phase II metabolites were not observed. Table 6 provides chemical structures of VAQ996 and proposed metabolite ml and their occurrence in in vitro and in vivo samples.

TABLE 6

| Parent | | |
|---|---|---|
| Metabolite No | Chemical structure | Matrix |
| VAQ996 | 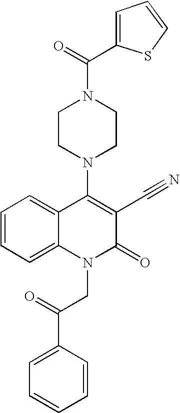 | In vitro:Liver microsomes ofmouse and humanIn vivo:mice: plasma, liver |
| m1( = VAR235) | 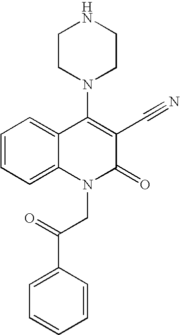 | In vitro:Liver microsomes ofmouse and humanMouse plasmaIn vivo:mice: plasma, liver |

TNF-alpha inhibitors and methods of preparing selected compounds suitable for use as TNF-alpha inhibitors or intermediates in the preparation thereof are disclosed in U.S. Pat. No. 7,105,519; U.S. Pat. No. 7,084,141; U.S. Pat. No. 7,157,469; U.S. Pat. No. 7,192,961; U.S. Pat. No. 7,129,236; U.S. Pat. No. 7,173,036; U.S. Patent Publication No. US-2005-0124604-AT; and U.S. Patent Publication No. US-2006-0229314-A1.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. Use of a compound in the manufacture of a medicament for the treatment of a disease or disorder which is mediated by TNF-alpha activity, the compound having a structure: 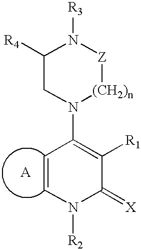
or a stereoisomer, or a pharmaceutically acceptable salt, ester, or solvate thereof, wherein:
A is a 5 to 7 membered ring having from 0 to 3 heteroatoms;
R₁ is selected from the group consisting of —CN, —NO, —NO₂, —C(═O)R₁₂, —C(═O)OR₁₂, —C(═O)NR₁₀R₁₁, —NR₁₂C(═O)R₁₂, —SO₂NR₁₀R₁₁, —NR₁₂SO₂R₁₂, and —S(O)ₘR₁₂, wherein m is from 0 to 3;
R₂ is selected from the group consisting of hydrogen, alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, heterocyclyl, substituted heterocyclyl, heterocyclylalkyl, substituted heterocyclylalkyl, heterocyclylaryl, substituted heterocyclylaryl, —(CH₂)ₓC(═O)aryl, substituted —(CH₂)ₓC(═O)aryl, —(CH₂)ₓC(═O)heterocyclyl, substituted —(CH₂)ₓC(═O)heterocyclyl, —(CH₂)ₓC(═O)heterocyclylalkyl, substituted —(CH₂)ₓC(═O)heterocyclylalkyl, —(CH₂)ₓC(═O)heterocyclylaryl, substituted —(CH₂)ₓC(═O)heterocyclylaryl, and —(CH₂)ₓNR₁₀R₁₁, wherein x is from 1 to 4;
R₃ is selected from the group consisting of hydrogen, alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, heterocyclyl, substituted heterocyclyl, heterocyclylalkyl, substituted heterocyclylalkyl, heterocyclylaryl, substituted heterocyclylaryl, formyl, acetyl, and —(C═O)R₁₂;
R₄ is selected from the group consisting of hydrogen, halogen, —R₁₂, —OR₁₂, —SR₁₂, and —NR₁₀R₁₁;
R₁₀ and R₁₁ are independently selected from the group consisting of hydrogen, alkyl, alkenyl, cycloalkyl, aryl, alkylaryl, arylalkyl, acylalkyl, heterocyclyl, heterocyclylalkyl, and heterocyclylaryl, or R₁₀ and R₁₁ together with the nitrogen atom to which they are attached comprise a heterocycle or a substituted heterocycle;
R₁₂ is independently selected from the group consisting of elected from the group consisting of hydrogen, alkyl, alkenyl, cycloalkyl, aryl, alkylaryl, arylalkyl, acylalkyl, heterocyclyl, heterocyclylalkyl, and heterocyclylaryl;
X is selected from the group consisting of O and S;
Z is selected from the group consisting of —C(═O)— and —CHR₁₂—; and
n is 0, 1 or 2, with the proviso that when n is 0, Z is —C(═O)—.

2. The use of claim 1, wherein the compound has a formula: 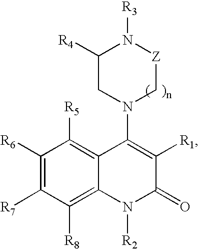
or a stereoisomer, or a pharmaceutically acceptable salt, ester, or solvate thereof, wherein:
R₁, R₂, R₃, R₄, Z, and n are as defined above; and wherein
R₅, R₆, R₇, and R₈ are independently selected from the group consisting of hydrogen, halogen, —R₁₂, —OR₁₂, —SR₁₂, and —NR₁₀R₁₁.

3. The use of claim 1, wherein the compound has a formula selected from the group consisting of: 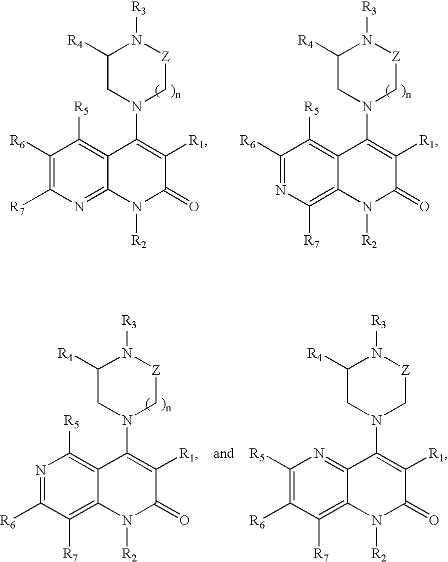
or a stereoisomer, or a pharmaceutically acceptable salt, ester, or solvate thereof, wherein:
R₁, R₂, R₃, R₄, Z, and n are as defined above; and wherein
R₅, R₆, and R₇ are independently selected from the group consisting of hydrogen, halogen, —R₁₂, —OR₁₂, —SR₁₂, and —NR₁₀R₁₁.

4. The use of claim 1, wherein the compound has a formula selected from the group consisting of 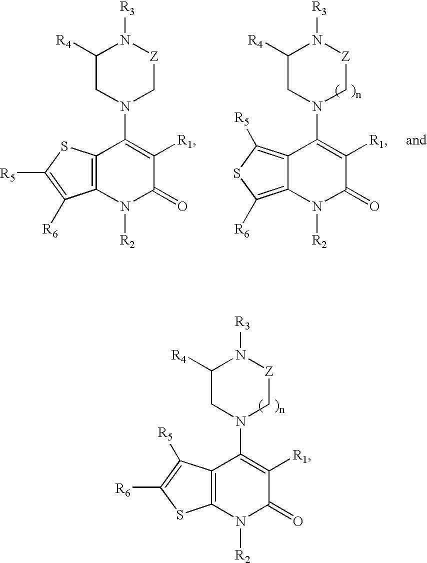
or a stereoisomer, or a pharmaceutically acceptable salt, ester, or solvate thereof, wherein:
R₁, R₂, R₃, R₄, Z, and n are as defined above; and wherein
R₅ and R₆ are independently selected from the group consisting of hydrogen, halogen, —R₁₂, —OR₁₂, —SR₁₂, and —NR₁₀R₁₁.

5. The use of claim 1, wherein A is a 5 to 6 membered ring having a heteroatom selected from the group consisting of N and S; wherein R₁ is selected from the group consisting of —NO₂, —C(═O)R₁₂, —C(═O)OR₁₂, and —C(═O)NR₁₀R₁₁; and wherein R₂ is selected from the group consisting of R₂ is selected from the group consisting of alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, —(CH₂)ₓC(═O)aryl, and substituted —(CH₂)ₓC(═O)aryl.

6. The use of claim 1, wherein the compound is selected from the group consisting of:

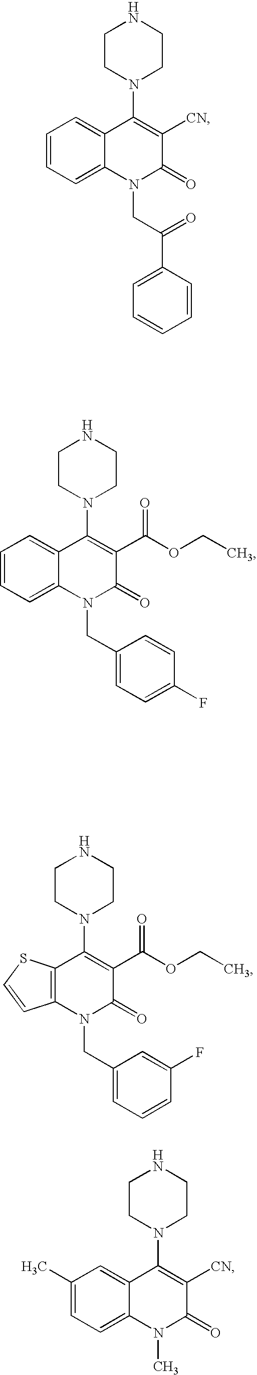

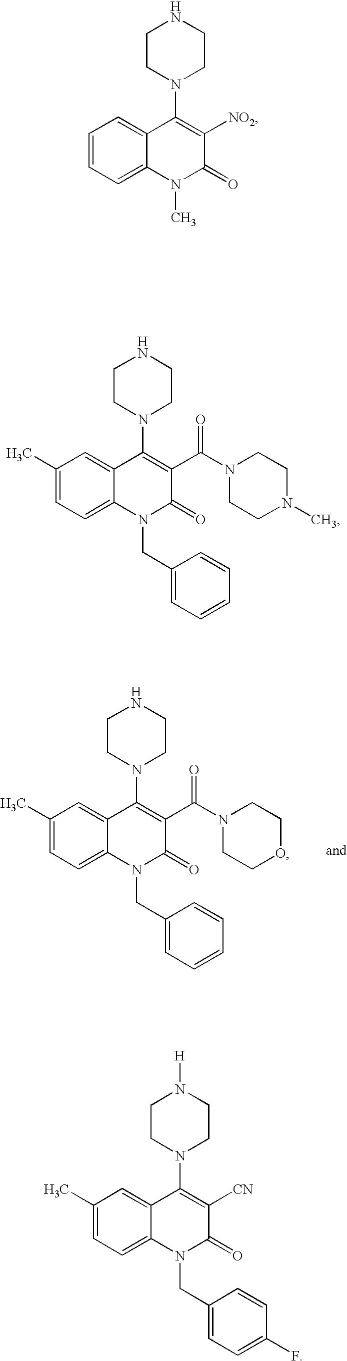

7. The use according to any one of claims 1 to 6, wherein the compound is in a form of a salt.

8. The use according to any one of claims 1 to 6, wherein the compound is a pharmaceutical.

9. The use according to any one of claims 1 to 6, wherein the compound is in association with at least one pharmaceutically acceptable excipient.

10. A method of treating a disease or disorder which is mediated by TNF-alpha activity, the method comprising administering to a subject in need of such treatment an effective amount of a compound having a structure: 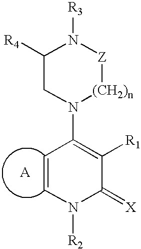
or a stereoisomer, or a pharmaceutically acceptable salt, ester, or solvate thereof, wherein:
A is a 5 to 7 membered ring having from 0 to 3 heteroatoms;
R₁ is selected from the group consisting of —CN, —NO, —NO₂, —C(═O)R₁₂, —C(═O)OR₁₂, —C(═O)NR₁₀R₁₁, —NR₁₂C(═O)R₁₂, —SO₂NR₁₀R₁₁, —NR₁₂SO₂R₁₂, and —S(O)ₘR₁₂, wherein m is from 0 to 3;
R₂ is selected from the group consisting of hydrogen, alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, heterocyclyl, substituted heterocyclyl, heterocyclylalkyl, substituted heterocyclylalkyl, heterocyclylaryl, substituted heterocyclylaryl, —(CH₂)ₓC(═O)aryl, substituted —(CH₂)ₓC(═O)aryl, —(CH₂)ₓC(═O)heterocyclyl, substituted —(CH₂)ₓC(═O)heterocyclyl, —(CH₂)ₓC(═O)heterocyclylalkyl, substituted —(CH₂)ₓC(═O)heterocyclylalkyl, —(CH₂)ₓC(═O)heterocyclylaryl, substituted —(CH₂)ₓC(═O)heterocyclylaryl, and —(CH₂)ₓNR₁₀R₁₁, wherein x is from 1 to 4;
R₃ is selected from the group consisting of hydrogen, alkyl, substituted alkyl, alkenyl, substituted alkenyl, cycloalkyl, substituted cycloalkyl, aryl, substituted aryl, alkylaryl, substituted alkylaryl, arylalkyl, substituted arylalkyl, acylalkyl, substituted acylalkyl, heterocyclyl, substituted heterocyclyl, heterocyclylalkyl, substituted heterocyclylalkyl, heterocyclylaryl, substituted heterocyclylaryl, formyl, acetyl, and —(C═O)R₁₂;
is selected from the group consisting of hydrogen, halogen, —R₁₂, —OR₁₂, —SR₁₂, and —NR₁₀R₁₁;
R₁₀ and R₁₁ are independently selected from the group consisting of hydrogen, alkyl, alkenyl, cycloalkyl, aryl, alkylaryl, arylalkyl, acylalkyl, heterocyclyl, heterocyclylalkyl, and heterocyclylaryl, or R₁₀ and R₁₁ together with the nitrogen atom to which they are attached comprise a heterocycle or a substituted heterocycle;
R₁₂ is independently selected from the group consisting of elected from the group consisting of hydrogen, alkyl, alkenyl, cycloalkyl, aryl, alkylaryl, arylalkyl, acylalkyl, heterocyclyl, heterocyclylalkyl, and heterocyclylaryl;
X is selected from the group consisting of O and S;
Z is selected from the group consisting of —C(═O)— and —CHR₁₂—; and
n is 0, 1 or 2, with the proviso that when n is 0, Z is —C(═O)—.

11. The method of claim 10, wherein the disease or disorder is inflammation.

12. The method of claim 10, wherein the disease or disorder is septic shock.

13. The method of claim 10, wherein the disease or disorder is arthritis.

14. The method of claim 10, wherein the disease or disorder is cancer.

15. The method of claim 10, wherein the disease or disorder is acute respiratory distress syndrome.

16. The method of claim 10, wherein the disease or disorder is an inflammatory disease.

17. The method of claim 16, wherein the inflammatory disease is selected from the group consisting of rheumatoid arthritis, osteoarthritis, inflammatory bowel disease, and asthma.

18. The method of claim 10, wherein the disease or disorder is an autoimmune disorder.

19. The method of claim 18, wherein the autoimmune disorder is selected from the group consisting of diabetes, asthma, and multiple sclerosis.

20. The method of claim 10, wherein the disease or disorder is a disease associated with excess glucocorticoid levels.

21. The method of claim 20, wherein the disease is Cushing's disease.
